SEQUENCE LISTING (186 sequences)

SEQ ID NO: 1 - protein, 1368 aa, Artificial Sequence [sequence omitted]

SEQ ID NO: 2 - protein, 84 aa, Artificial Sequence
  Uracil-DNA glycosylase inhibitor (UGI)
MTNLSDIIEKETGKQLVIQESILMLPEEVEEVIGNKPESDILVHTAYDESTDENVMLLTSDAPEYKPWALVIQDSNGENKIKML

SEQ ID NO: 3 - DNA, 93 bp, Artificial Sequence
  exemplary hybrid crRNAtracrRNA, gRNA sequence
guuuaagagcuaugcuggaaacagcauagcaaguuuaaauaaggcuaguccguuaucaacuugaaaaaguggcaccgagucggugcuuuuuuu

SEQ ID NO: 4 - RNA, 79 nt, Artificial Sequence
  tracrRNA 1
ggaaccauucaaaacagcauagcaaguuaaaauaaggcuaguccguuaucaacuugaaaaaguggcaccgagucggugc

SEQ ID NO: 5 - RNA, 60 nt, Artificial Sequence
  tracrRNA 2
uagcaaguuaaaauaaggcuaguccguuaucaacuugaaaaaguggcaccgagucggugc

SEQ ID NO: 6 - RNA, 64 nt, Artificial Sequence
  tracrRNA 3
agcauagcaaguuaaaauaaggcuaguccguuaucaacuugaaaaaguggcaccgagucggugc

SEQ ID NO: 7 - RNA, 70 nt, Artificial Sequence
  tracrRNA 4
caaaacagcauagcaaguuaaaauaaggcuaguccguuaucaacuugaaaaaguggcaccgagucggugc

SEQ ID NO: 8 - RNA, 45 nt, Artificial Sequence
  tracrRNA 5
uagcaaguuaaaauaaggcuaguccguuaucaacuugaaaaagug

SEQ ID NO: 9 - RNA, 32 nt, Artificial Sequence
  tracrRNA 6
uagcaaguuaaaauaaggcuaguccguuauca

SEQ ID NO: 10 - RNA, 26 nt, Artificial Sequence
  tracrRNA 7
uagcaaguuaaaauaaggcuaguccg

SEQ ID NO: 11 - RNA, 66 nt, Artificial Sequence
  Telomerase Ku Binding Motif
uucuugucguacuuauagaucgcuacguuauuucaauuuugaaaaucugaguccugggagugcgga

SEQ ID NO: 12 - protein, 1094 aa, Artificial Sequence
  Telomerase Ku Heterodimer
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQDMVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 13 - DNA, 10 bp, Artificial Sequence
  Telomerase Sm7 consensus site (single stranded)
aauuuuugga

SEQ ID NO: 14 - protein, 83 aa, Artificial Sequence
  Monomeric Sm-like protein (archaea)
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 15 - DNA, 19 bp, Artificial Sequence
  MS2 phage operator stem loop
acaugaggaucacccaugu

SEQ ID NO: 16 - protein, 117 aa, Artificial Sequence
  MS2 coat protein
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 17 - DNA, 26 bp, Artificial Sequence
  PP7 phage operator stem loop
auaaggaguuuauauggaaacccuua

SEQ ID NO: 18 - protein, 128 aa, Artificial Sequence
  PP7 coat protein (PCP)
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 19 - DNA, 19 bp, Artificial Sequence
  SfMu Com stem loop
cugaaugccugcgagcauc

SEQ ID NO: 20 - protein, 62 aa, Artificial Sequence
  SfMu Com binding protein
MKSIRCKNCNKLLFKADSFDHIEIRCPRCKRHIIMLNACEHPTEKHCGKREKITHSDETVRY

SEQ ID NO: 21 - RNA, 25 nt, Artificial Sequence
  4nt MS2 extension
  misc_feature (1)..(2): GC linker
  misc_feature (3)..(4): Extension
  misc_feature (4)..(23): MS2 aptamer
  misc_feature (24)..(25): Extension
gcgcacaugaggaucacccaugugc

SEQ ID NO: 22 - RNA, 31 nt, Artificial Sequence
  10nt MS2 extension
  misc_feature (1)..(2): Linker
  misc_feature (3)..(7): Extension
  misc_feature (8)..(26): MS2 Aptamer
  misc_feature (27)..(31): Extension
gcgagcgacaugaggaucacccaugucgcuc

SEQ ID NO: 23 - RNA, 37 nt, Artificial Sequence
  16 nt MS2 extension
  misc_feature (1)..(2): Linker
  misc_feature (3)..(10): Extension
  misc_feature (11)..(29): MS2 Aptamer
  misc_feature (30)..(37): Extension
gccacgagcgacaugaggaucacccaugucgcucgug

SEQ ID NO: 24 - RNA, 47 nt, Artificial Sequence
  26nt MS2 extension
  misc_feature (1)..(2): Linker
  misc_feature (3)..(15): Linker
  misc_feature (16)..(34): MS2 aptamer
  misc_feature (35)..(47): Extension
gccgucagacgagcgacaugaggaucacccaugucgcucgucugacg

SEQ ID NO: 25 - protein, 1368 aa, Artificial Sequence
  S. pyogenes dCas9 protein sequence
  MISC_FEATURE (10)..(10): Active site mutant D10A
  MISC_FEATURE (840)..(840): Active site mutant H840A
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 26 - protein, 1367 aa, Artificial Sequence
  Cas9 D10A Protein
  Mutation (10)..(10): D10A
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 27 - DNA, 4104 bp, Artificial Sequence
  DNA encoding Cas9 D10A Protein
  misc_feature (29)..(): A to C
  misc_feature (29)..(29): A to C
atggataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgac

SEQ ID NO: 28 - DNA, 128 bp, Artificial Sequence
  RNA scaffold expression cassette (S. pyogenes), containing a 20-nucleotide programmable sequence, a CRISPR RNA motif, and an MS2 operator motif
  misc_feature (1)..(20): 20-nucleotide programmable sequence
  misc_feature (21)..(96): CRISPR RNA motif (tracrRNA)
  misc_feature (97)..(98): GC linker
  misc_feature (99)..(100): Extension
  misc_feature (101)..(119): MS2 motif
  misc_feature (120)..(121): Extension
  misc_feature (122)..(128): Terminator
nnnnnnnnnnnnnnnnnnnngttttagagctagaaatagcaagttaaaataaggctagtccgttatcaacttgaaaaagtggcaccgagtcggtgcgcgcacatgaggatcacccatgtgcttttttt

SEQ ID NO: 29 - DNA, 150 bp, Artificial Sequence
  RNA scaffold containing two MS2 loops (2xMS2)
  misc_feature (83)..(101): MS2 scaffold
  misc_feature (112)..(130): MS2 scaffold
gttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttcccttttttt

SEQ ID NO: 30 - protein, 340 aa, Artificial Sequence
  Effector AID -MCP fusion
  MISC_FEATURE (1)..(198): AID
  MISC_FEATURE (199)..(223): MCP
  MISC_FEATURE (224)..(340): UGI
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 31 - DNA, 594 bp, Artificial Sequence
  wtAID cDNA
  misc_feature (112)..(114): Ser38 codon
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 32 - protein, 198 aa, Artificial Sequence [sequence omitted]

SEQ ID NO: 33 - DNA, 594 bp, Artificial Sequence
  AID_S38A cDNA
  misc_feature (112)..(114): S38A mutation
atggacagcctcttgatgaaccggaggaagtttctttaccaattcaaaaatgtccgctgggctaagggtcggcgtgagacctacctgtgctacgtagtgaagaggcgtgacgccgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactt

SEQ ID NO: 34 - protein, 198 aa, Artificial Sequence
  AID_S38A protein
  MISC_FEATURE (38)..(38): S38A mutation
MDSLLMNRRKFLYQFKNVRWAKGRRETYLCYVVKRRDAATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 35 - protein, 1835 aa, Artificial Sequence
  Protein sequence of ARNA scaffold mediated recruitment system nu construct
  MISC_FEATURE (1)..(7): Nuclear Localization Signal (NLS)
  MISC_FEATURE (8)..(205): AID
  MISC_FEATURE (206)..(230): Linker
  MISC_FEATURE (231)..(347): MCP
  MISC_FEATURE (351)..(368): T2A peptide
  MISC_FEATURE (371)..(1737): nCAS9D10A
  MISC_FEATURE (1742)..(1824): UGI
  MISC_FEATURE (1829)..(1835): Nuclear Localization Signal (NLS)
PKKKRKVMDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 36 - protein, 1941 aa, Artificial Sequence
  Protein sequence of ARNA scaffold mediated recruitment system nu.2 construct
  MISC_FEATURE (1)..(7): Nuclear Localization Signal (NLS)
  MISC_FEATURE (8)..(205): AID
  MISC_FEATURE (206)..(230): Linker
  MISC_FEATURE (231)..(347): MCP
  MISC_FEATURE (351)..(368): T2A peptide
  MISC_FEATURE (371)..(377): Nuclear Localization Signal (NLS)
  MISC_FEATURE (378)..(1744): nCAS9D10A
  MISC_FEATURE (1755)..(1837): UGI
  MISC_FEATURE (1848)..(1930): UGI
  MISC_FEATURE (1935)..(1941): Nuclear Localization Signal (NLS)
PKKKRKVMDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 37 - protein, 1866 aa, Artificial Sequence
  Protein sequence of RNA scaffold mediated recruitment system
  MISC_FEATURE (1)..(7): Nuclear Localization Signal (NLS)
  MISC_FEATURE (8)..(235): AID
  MISC_FEATURE (236)..(261): Linker
  MISC_FEATURE (262)..(378): MCP
  MISC_FEATURE (382)..(399): T2A peptide
  MISC_FEATURE (402)..(1768): nCAS9D10A
  MISC_FEATURE (1772)..(1855): UGI
  MISC_FEATURE (1860)..(1866): Nuclear Localization Signal (NLS)
PKKKRKVMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 38 - RNA, 128 nt, Artificial Sequence
  RNA sequence of gRNA_MS2 construct
  misc_feature (1)..(20): N represents the customisable target
  misc_feature (21)..(96): gRNA scaffold
  misc_feature (101)..(119): MS2 aptamer
nnnnnnnnnnnnnnnnnnnnguuuuagagcuagaaauagcaaguuaaaauaaggcuaguccguuaucaacuugaaaaaguggcaccgagucggugcgcgcacaugaggaucacccaugugcuuuuuuu

SEQ ID NO: 39 - DNA, 168 bp, Artificial Sequence
  sequence of gRNA_2xMS2 construct
  misc_feature (1)..(20): N represents customisable target sequence
  misc_feature (21)..(96): gRNA scaffold
  misc_feature (103)..(121): MS2 aptamers
  misc_feature (132)..(150): MS2 aptamers
  misc_feature (164)..(170): Terminator
nnnnnnnnnnnnnnnnnnguuuuagagcuagaaauagcaaguuaaaauaaggcuaguccguuaucaacuugaaaaaguggcaccgagucggugcgggagcacaugaggaucacccaugugccacgagcgacaugaggaucacccaugucgctcgtgttcccuuuuuuu

SEQ ID NO: 40 - DNA, 103 bp, Artificial Sequence
  S. pyrogenes sgRNA
  misc_feature (1)..(20): N represents the 20 nt target specific spacer sequence
  misc_feature (21)..(96): Constant sgRNA sequence
  misc_feature (97)..(103): Terminator
nnnnnnnnnnnnnnnnnnnngttttagagctagaaatagcaagttaaaataaggctagtccgttatcaacttgaaaaagtggcaccgagtcggtgcttttttt

SEQ ID NO: 41 - DNA, 170 bp, Artificial Sequence
  2xMS2_3′
  misc_feature (1)..(20): N represents the 20 nt target specific spacer sequence
  misc_feature (21)..(96): constant sgRNA sequence
  misc_feature (97)..(102): Extension
  misc_feature (103)..(121): MS2 (C5 variant)
  misc_feature (122)..(131): Extension
  misc_feature (132)..(150): MS2 (C5 variant)
  misc_feature (151)..(163): Extension
  misc_feature (164)..(170): Terminator
nnnnnnnnnnnnnnnnnnnngttttagagctagaaatagcaagttaaaataaggctagtccgttatcaacttgaaaaagtggcaccgagtcggtgcgggagcacatgaggatcacccatgtgccacgagcgacatgaggatcacccatgtcgctcgtgttcccttttttt

SEQ ID NO: 42 - DNA, 133 bp, Artificial Sequence
  1xMS2_TL
  misc_feature (1)..(20): N represents the 20 nt target specific spacer sequence
  misc_feature (21)..(32): constant sgRNA sequence
  misc_feature (33)..(37): Extension
  misc_feature (38)..(56): MS2 (C5 variant) aptamer
  misc_feature (57)..(66): Extension
  misc_feature (67)..(126): constant sgRNA sequence
  misc_feature (127)..(133): Terminator
nnnnnnnnnnnnnnnnnnnngttttagagctaggccaacatgaggatcacccatgtctgcagggcctagcaagttaaaataaggctagtccgttatcaacttgaaaaagtggcaccgagtcggtgcttttttt

SEQ ID NO: 43 - DNA, 128 bp, Artificial Sequence; identical to SEQ ID NO: 28
  1xMS2_3′
  misc_feature (1)..(20): N respresents the 20 nt target specific spacer sequence
  misc_feature (21)..(96): constant sgRNA sequence
  misc_feature (97)..(100): Extension
  misc_feature (101)..(119): MS2 (C5 variant) aptamer
  misc_feature (120)..(121): Extension
  misc_feature (122)..(128): Terminator

SEQ ID NO: 44 - DNA, 117 bp, Artificial Sequence
  7bp Extended_US
  misc_feature (1)..(20): N represents the 20 nt target specific spacer sequence
  misc_feature (21)..(32): constant sgRNA sequence
  misc_feature (33)..(39): extended repeat anti-repeat sequences
  misc_feature (40)..(43): constant sgRNA sequence
  misc_feature (44)..(50): extended repeat anti-repeat sequences
  misc_feature (51)..(110): constant sgRNA sequence
  misc_feature (111)..(117): Terminator
nnnnnnnnnnnnnnnnnnnngttttagagctatgctgttgaaaaacagcatagcaagttaaaataaggctagtccgttatcaacttgaaaaagtggcaccgagtcggtgcttttttt

SEQ ID NO: 45 - DNA, 184 bp, Artificial Sequence
  2xMS2_3′ 7bp_extended_US
  misc_feature (1)..(20): N represents the 20 nt target specific spacer sequence
  misc_feature (21)..(32): constant sgRNA sequence
  misc_feature (33)..(39): extended repeat anti-repeat sequences
  misc_feature (40)..(43): constant sgRNA sequence
  misc_feature (44)..(50): extended repeat anti-repeat sequences
  misc_feature (51)..(110): constant sgRNA sequence
  misc_feature (111)..(116): Extension
  misc_feature (117)..(135): MS2 (C5 variant)
  misc_feature (136)..(145): Extension
  misc_feature (146)..(164): MS2 (C5 variant) aptamer
  misc_feature (165)..(177): Extension
  misc_feature (178)..(184): Terminator
nnnnnnnnnnnnnnnnnnnngttttagagctatgctgttgaaaaacagcatagcaagttaaaataaggctagtccgttatcaacttgaaaaagtggcaccgagtcggtgcgggagcacatgaggatcacccatgtgccacgagcgacatgaggatcacccatgtcgctcgtgttcccttttttt

SEQ ID NO: 46 - DNA, 147 bp, Artificial Sequence
  1xMS2-TL _7bp extended_US
  misc_feature (1)..(20): N represents the  20 nt target specific spacer sequence
  misc_feature (21)..(32): constant sgRNA sequence
  misc_feature (33)..(39): extended repeat anti-repeat sequences
  misc_feature (40)..(44): Extension
  misc_feature (45)..(63): MS2 (C5 variant) aptamer
  misc_feature (64)..(73): Extension
  misc_feature (74)..(80): extended repeat anti-repeat sequences
  misc_feature (81)..(140): constant sgRNA sequence
  misc_feature (141)..(147): constant sgRNA sequence
  misc_feature (141)..(147): Terminator
nnnnnnnnnnnnnnnnnnnngttttagagctatgctgttggccaacatgaggatcacccatgtctgcagggccaacagcatagcaagttaaaataaggctagtccgttatcaacttgaaaaagtggcaccgagtcggtgcttttttt

SEQ ID NO: 47 - DNA, 132 bp, Artificial Sequence
  1xMS2-3′_2bp extended_US
  misc_feature (1)..(20): N represents the the 20 nt target specific spacer sequence
  misc_feature (21)..(32): constant sgRNA sequence
  misc_feature (33)..(34): extended repeat anti-repeat sequences
  misc_feature (35)..(38): constant sgRNA sequence
  misc_feature (39)..(40): extended repeat anti-repeat sequences
  misc_feature (41)..(100): constant sgRNA sequence
  misc_feature (101)..(104): Extension
  misc_feature (105)..(123): MS2 (C5 variant) aptamer
  misc_feature (124)..(126): Extension
  misc_feature (127)..(132): Terminator
nnnnnnnnnnnnnnnnnnnngttttagagctatggaaacatagcaagttaaaataaggctagtccgttatcaacttgaaaaagtggcaccgagtcggtgcgcgcacatgaggatcacccatgtgcttttttt

SEQ ID NO: 48 - DNA, 138 bp, Artificial Sequence
  1xMS2-3′_5bp-extended_US
  misc_feature (1)..(20): N represents the 20 nt target specific spacer sequence
  misc_feature (21)..(32): constant sgRNA sequence
  misc_feature (33)..(37): extended repeat anti-repeat sequences
  misc_feature (38)..(41): constant sgRNA sequence
  misc_feature (42)..(46): extended repeat anti-repeat sequences
  misc_feature (47)..(106): constant sgRNA sequence
  misc_feature (107)..(110): Extension
  misc_feature (111)..(129): MS2 (C5 variant) aptamer
  misc_feature (130)..(131): Extension
  misc_feature (132)..(138): Terminator
nnnnnnnnnnnnnnnnnnnngttttagagctatgctggaaacagcatagcaagttaaaataaggctagtccgttatcaacttgaaaaagtggcaccgagtcggtgcgcgcacatgaggatcacccatgtgcttttttt

SEQ ID NO: 49 - DNA, 142 bp, Artificial Sequence
  1xMS2-3′_7bp-extended_US
  misc_feature (1)..(20): N denotes the 20 nt target specific spacer sequence
  misc_feature (21)..(32): constant sgRNA sequence
  misc_feature (33)..(39): extended repeat anti-repeat sequences
  misc_feature (40)..(43): constant sgRNA sequence
  misc_feature (44)..(50): extended repeat anti-repeat sequences
  misc_feature (51)..(110): constant sgRNA sequence
  misc_feature (111)..(114): Extensions
  misc_feature (111)..(133): MS2 (C5 variant) aptamer
  misc_feature (134)..(135): Extension
  misc_feature (136)..(142): Extension
  misc_feature (136)..(142): Terminator
nnnnnnnnnnnnnnnnnnnngttttagagctatgctgttgaaaaacagcatagcaagttaaaataaggctagtccgttatcaacttgaaaaagtggcaccgagtcggtgcgcgcacatgaggatcacccatgtgcttttttt

SEQ ID NO: 50 - DNA, 148 bp, Artificial Sequence
  1xMS2-3′_10bp-extended_US
  misc_feature (1)..(20): N denotes the 20 nt target specific spacer sequence
  misc_feature (21)..(32): constant sgRNA sequence
  misc_feature (33)..(42): extended repeat anti-repeat sequences
  misc_feature (43)..(46): constant sgRNA sequence
  misc_feature (47)..(56): extended repeat anti-repeat sequences
  misc_feature (57)..(116): constant sgRNA sequence
  misc_feature (117)..(120): Extension
  misc_feature (121)..(139): MS2 (C5 variant) aptamer
  misc_feature (140)..(141): Extension
  misc_feature (142)..(148): Terminator
nnnnnnnnnnnnnnnnnnnngttttagagctatgctgttttggaaacaaaacagcatagcaagttaaaataaggctagtccgttatcaacttgaaaaagtggcaccgagtcggtgcgcgcacatgaggatcacccatgtgcttttttt

SEQ ID NO: 51 - DNA, 147 bp, Artificial Sequence
  1xMS2-SL2_7bp-extended_US
  misc_feature (1)..(20): N denotes the 20 nt target specific spacer sequence
  misc_feature (21)..(32): constant sgRNA sequence
  misc_feature (33)..(39): extended repeat anti-repeat sequences
  misc_feature (40)..(43): constant sgRNA sequence
  misc_feature (44)..(50): the extended repeat anti-repeat sequences
  misc_feature (51)..(86): constant sgRNA sequence
  misc_feature (87)..(91): Extension
  misc_feature (92)..(110): MS2 (C5 variant) aptamer
  misc_feature (111)..(120): Extension
  misc_feature (121)..(140): constant sgRNA sequence
  misc_feature (141)..(147): Terminator
nnnnnnnnnnnnnnnnnnnngttttagagctatgctgttgaaaaacagcatagcaagttaaaataaggctagtccgttatcaacttggccaacatgaggatcacccatgtctgcagggccaagtggcaccgagtcggtgcttttttt

SEQ ID NO: 52 - DNA, 167 bp, Artificial Sequence
  2xMS2_C5-SL2_f6-3′_7bp extended-US
  misc_feature (1)..(20): N denotes the 20 nt target specific spacer sequence
  misc_feature (21)..(32): constant sgRNA sequence
  misc_feature (33)..(39): extended repeat anti-repeat sequences
  misc_feature (40)..(43): constant sgRNA sequence
  misc_feature (44)..(50): extended repeat anti-repeat sequences
  misc_feature (51)..(86): constant sgRNA sequence
  misc_feature (87)..(91): Extension
  misc_feature (92)..(110): f6 aptamer
  misc_feature (111)..(120): Extension
  misc_feature (121)..(140): constant sgRNA sequence
  misc_feature (141)..(144): Extension
  misc_feature (145)..(158): f6 aptamer
  misc_feature (159)..(160): Extension
  misc_feature (161)..(167): Terminator
nnnnnnnnnnnnnnnnnnnngttttagagctatgctgttgaaaaacagcatagcaagttaaaataaggctagtccgttatcaacttggccaacatgaggatcacccatgtctgcagggccaagtggcaccgagtcggtgcgcgcccacagtcactggggcttttttt

SEQ ID NO: 53 - DNA, 20 bp, Artificial Sequence
  sgRNa target site sequence Site2
gaacacaaagcatagactgc

SEQ ID NO: 54 - DNA, 20 bp, Artificial Sequence
  sgRNa target site sequence Site 3
ggcccagactgagcacgtga

SEQ ID NO: 55 - DNA, 20 bp, Artificial Sequence
  sgRNa target site sequence CTNNB1
ctggactctggaatccattc

SEQ ID NO: 56 - DNA, 20 bp, Artificial Sequence
  sgRNa target site sequence EGFR
atcacgcagctcatgccctt

SEQ ID NO: 57 - DNA, 20 bp, Artificial Sequence
  sgRNa target site sequence PCSK9
caggttccacgggatgctct

SEQ ID NO: 58 - DNA, 20 bp, Artificial Sequence
  sgRNa target site sequence FANCF
ggaatcccttctgcagcacc

SEQ ID NO: 59 - DNA, 20 bp, Artificial Sequence
  sgRNa target site sequence TRAC
ttcgtatctgtaaaaccaag

SEQ ID NO: 60 - DNA, 20 bp, Artificial Sequence
  sgRNa target site sequence B2M
cttaccccacttaactatct

SEQ ID NO: 61 - DNA, 20 bp, Artificial Sequence
  sgRNA target site sequences CR0118_PDCD1
cagttccaaaccctggtggt

SEQ ID NO: 62 - DNA, 20 bp, Artificial Sequence
  sgRNA target site sequences  CR0107_PDCD1
gggggttccagggcctgtct

SEQ ID NO: 63 - DNA, 20 bp, Artificial Sequence
  sgRNA target site sequences CR0057-TRAC_EX3
ttcgtatctgtaaaaccaag

SEQ ID NO: 64 - DNA, 20 bp, Artificial Sequence
  sgRNA target site sequences CR0151_CD2
gttcagccaaaacctcccca

SEQ ID NO: 65 - DNA, 20 bp, Artificial Sequence
  sgRNA target site sequences CR0121_PDCD1
ggagtctgagagatggagag

SEQ ID NO: 66 - DNA, 20 bp, Artificial Sequence
  sgRNA target site sequences CR0165_CIITA
cagctcacagtgtgccacca

SEQ ID NO: 67 - DNA, 20 bp, Artificial Sequence
  sgRNA target site sequences TRAC_22550571
ttcaaaacctgtcagtgatt

SEQ ID NO: 68 - DNA, 20 bp, Artificial Sequence
  sgRNA target site sequences PDCD1_241852953
gggggttccagggcctgtct

SEQ ID NO: 69 - DNA, 20 bp, Artificial Sequence
  sgRNA target site sequences CTNNB1
ctggactctggaatccattc

SEQ ID NO: 70 - DNA, 20 bp, Artificial Sequence
  Site2 Forward Primer
tggcccttcaagttactgca

SEQ ID NO: 71 - DNA, 23 bp, Artificial Sequence
  Site2 Reverse Primer
agcacatgacagttaaggtttgt

SEQ ID NO: 72 - DNA, 20 bp, Artificial Sequence
  Site3 Forward Primer
aaacgcccatgcaattagtc

SEQ ID NO: 73 - DNA, 20 bp, Artificial Sequence
  Site3 Reverse Primer
agcccctgtctaggaaaagc

SEQ ID NO: 74 - DNA, 24 bp, Artificial Sequence
  CTNNB1 Forward Primer
caatgggtcatatcacagattctt

SEQ ID NO: 75 - DNA, 23 bp, Artificial Sequence
  CTNNB1 Reverse Primer
ccagctacttgttcttgagtgaa

SEQ ID NO: 76 - DNA, 20 bp, Artificial Sequence
  EGFR Forward Primer
tcatgcgtcttcacctggaa

SEQ ID NO: 77 - DNA, 20 bp, Artificial Sequence
  EGFR Reverse Primer
cgcacacacatatccccatg

SEQ ID NO: 78 - DNA, 20 bp, Artificial Sequence
  PCSK9 Forward Primer
cactagcagggacaaggtgg

SEQ ID NO: 79 - DNA, 20 bp, Artificial Sequence
  PCSK9 Reverse Primer
attcagctcagatggggtgg

SEQ ID NO: 80 - DNA, 20 bp, Artificial Sequence
  FANCF Forward Primer
cgctgggagattgacatgca

SEQ ID NO: 81 - DNA, 20 bp, Artificial Sequence
  FANCF Reverse Primer
ctcttgcctccactggttgt

SEQ ID NO: 82 - DNA, 20 bp, Artificial Sequence
  TRAC Forward Primer
acctaccccatccccagaag

SEQ ID NO: 83 - DNA, 20 bp, Artificial Sequence
  TRAC Reverse Primer
tccctaaaccccactcccag

SEQ ID NO: 84 - DNA, 21 bp, Artificial Sequence
  B2M Forward Primer
tgggtttcatccatccgacat

SEQ ID NO: 85 - DNA, 21 bp, Artificial Sequence
  B2M Reverse Primer
atgggatgggactcattcagg

SEQ ID NO: 86 - DNA, 42 bp, Artificial Sequence
  Synthetic crRNA Sequence
  misc_feature (1)..(20): N denotes the 20 nt target specific spacer sequence
  misc_feature (1)..(1): 2′OMe  (2′-O-Methyl nucleotides) and phosphorothioate modified residue
  misc_feature (2)..(2): 2′OMe  (2′-O-Methyl nucleotides) and phosphorothioate modified residue
nnnnnnnnnnnnnnnnnnnnguuuuagagcuaugcuguuuug

SEQ ID NO: 87 - DNA, 99 bp, Artificial Sequence
  Synthetic 1xMS2 tracrRNA-Aptamer Sequence
  misc_feature (97)..(98): 2′OMe (m) and phosphorothioate (*) modified residues
  misc_feature (97)..(98): 2′OMe and phosphorothioate modified residues
aacagcauagcaaguuaaaauaaggcuaguccguuaucaacuugaaaaaguggcaccgagucggugcgcgcacaugaggaucacccaugugcuuuuuuu

SEQ ID NO: 88 - DNA, 141 bp, Artificial Sequence
  Synthetic 2xMS2 tracrRNA-Aptamer Sequence
  misc_feature (139)..(140): 2′OMe and phosphorothioate modified residue
  misc_feature (139)..(140): 2′OMe and phosphorothioate modified residues
aacagcauagcaaguuaaaauaaggcuaguccguuaucaacuugaaaaaguggcaccgagucggugcgggagcacaugaggaucacccaugugccacgagcgacaugaggaucacccaugucgcucguguucccuuuuuuu

SEQ ID NO: 89 - DNA, 167 bp, Artificial Sequence
  Lentiviral sgRNA sequences
  misc_feature (1)..(20): n′s denote the 20 base target specific sequence
  misc_feature (1)..(2): residues 1 and 2 are phosphothorothioate modified
  misc_feature (1)..(2): residues 1 and 2 are 2′OMe modified
  misc_feature (168)..(169): residues 168 and 169 are phosphorothioate modified
  misc_feature (168)..(169): residues 168 and 169 are 2′OMe modified
nnnnnnnnnnnnnnnnnnnnguuuuagagcuagaaauagcaaguuaaaauaaggcuaguccguuaucaacuugaaaaaguggcaccgagucggugcgggagcacaugaggaucacccaugugccacgagcgacaugaggaucacccaugucgcucguguucccuuuu

SEQ ID NO: 90 - DNA, 20 bp, Artificial Sequence
  B2M_1 sgRNA guide sequence
cacagcccaagatagttaag

SEQ ID NO: 91 - DNA, 20 bp, Artificial Sequence
  B2M_2 sgRNA guide sequence
acagcccaagatagttaagt

SEQ ID NO: 92 - DNA, 20 bp, Artificial Sequence
  B2M_3 sgRNA guide sequence
ttaccccacttaactatctt

SEQ ID NO: 93 - DNA, 20 bp, Artificial Sequence
  B2M_4 sgRNA guide sequence
cttaccccacttaactatct

SEQ ID NO: 94 - DNA, 20 bp, Artificial Sequence
  B2M_5 sgRNA guide sequence
actcacgctggatagcctcc

SEQ ID NO: 95 - DNA, 20 bp, Artificial Sequence
  B2M_6 sgRNA guide sequence
ttggagtacctgaggaatat

SEQ ID NO: 96 - DNA, 20 bp, Artificial Sequence
  B2M_7 sgRNA guide sequence
tcgatctatgaaaaagacag

SEQ ID NO: 97 - DNA, 20 bp, Artificial Sequence
  B2M_8 sgRNA guide sequence
aacctgaaaagaaaagaaaa

SEQ ID NO: 98 - DNA, 20 bp, Artificial Sequence
  CD52_1 sgRNA guide sequence
gtacaggtaagagcaacgcc

SEQ ID NO: 99 - DNA, 20 bp, Artificial Sequence
  CD52_2 sgRNA guide sequence
ctcctcctacagatacaaac

SEQ ID NO: 100 - DNA, 20 bp, Artificial Sequence
  CD52_3 sgRNA guide sequence
cagatacaaactggactctc

SEQ ID NO: 101 - DNA, 20 bp, Artificial Sequence
  CD52_4 sgRNA guide sequence
ctcttacctgtaccataacc

SEQ ID NO: 102 - DNA, 20 bp, Artificial Sequence
  CD52_5 sgRNA guide sequence
gtatctgtaggaggagaagt

SEQ ID NO: 103 - DNA, 20 bp, Artificial Sequence
  CD52_6 sgRNA guide sequence
tgtatctgtaggaggagaag

SEQ ID NO: 104 - DNA, 20 bp, Artificial Sequence
  CD52_7 sgRNA guide sequence
gtccagtttgtatctgtagg

SEQ ID NO: 105 - DNA, 20 bp, Artificial Sequence
  TRAC_1 sgRNA guide sequence
aacaaatgtgtcacaaagta

SEQ ID NO: 106 - DNA, 20 bp, Artificial Sequence
  TRAC_2 sgRNA guide sequence
cttcttccccagcccaggta

SEQ ID NO: 107 - DNA, 20 bp, Artificial Sequence
  TRAC_3 sgRNA guide sequence
ttcttccccagcccaggtaa

SEQ ID NO: 108 - DNA, 20 bp, Artificial Sequence
  TRAC_4 sgRNA guide sequence
agcccaggtaagggcagctt

SEQ ID NO: 109 - DNA, 20 bp, Artificial Sequence
  TRAC_5 sgRNA guide sequence
tttcaaaacctgtcagtgat

SEQ ID NO: 110 - DNA, 20 bp, Artificial Sequence
  TRAC_6 sgRNA guide sequence
ttcaaaacctgtcagtgatt

SEQ ID NO: 111 - DNA, 20 bp, Artificial Sequence
  TRAC_7 sgRNA guide sequence
ccgaatcctcctcctgaaag

SEQ ID NO: 112 - DNA, 20 bp, Artificial Sequence
  TRAC_8 sgRNA guide sequence
cttacctgggctggggaaga

SEQ ID NO: 113 - DNA, 20 bp, Artificial Sequence
  TRAC_9 sgRNA guide sequence
ttcgtatctgtaaaaccaag

SEQ ID NO: 114 - DNA, 20 bp, Artificial Sequence
  TRBC1/2_1 sgRNA guide sequence
ccacacccaaaaggccacac

SEQ ID NO: 115 - DNA, 20 bp, Artificial Sequence
  TRBC1/2_2 sgRNA guide sequence
cccaccagctcagctccacg

SEQ ID NO: 116 - DNA, 20 bp, Artificial Sequence
  TRBC1/2_3 sgRNA guide sequence
cgctgtcaagtccagttcta

SEQ ID NO: 117 - DNA, 20 bp, Artificial Sequence
  TRBC1/2_4 sgRNA guide sequence
gctgtcaagtccagttctac

SEQ ID NO: 118 - DNA, 20 bp, Artificial Sequence
  TRBC1/2_5 sgRNA guide sequence
agtccagttctacgggctct

SEQ ID NO: 119 - DNA, 20 bp, Artificial Sequence
  TRBC1/2_6 sgRNA guide sequence
cacccagatcgtcagcgccg

SEQ ID NO: 120 - DNA, 20 bp, Artificial Sequence
  TRBC1/2_7 sgRNA guide sequence
acctgctctaccccaggcct

SEQ ID NO: 121 - DNA, 20 bp, Artificial Sequence
  TRBC1/2_8 sgRNA guide sequence
ccactcacctgctctacccc

SEQ ID NO: 122 - DNA, 20 bp, Artificial Sequence
  TRBC1_1 sgRNA guide sequence
cacggacccgcagcccctca

SEQ ID NO: 123 - DNA, 20 bp, Artificial Sequence
  TRBC1_2 sgRNA guide sequence
gcgggggttctgccagaagg

SEQ ID NO: 124 - DNA, 20 bp, Artificial Sequence
  TRBC1_3 sgRNA guide sequence
gttgcgggggttctgccaga

SEQ ID NO: 125 - DNA, 20 bp, Artificial Sequence
  TRBC1_4 sgRNA guide sequence
atgacgagtggacccaggat

SEQ ID NO: 126 - DNA, 20 bp, Artificial Sequence
  TRBC1_5 sgRNA guide sequence
tgacgagtggacccaggata

SEQ ID NO: 127 - DNA, 20 bp, Artificial Sequence
  TRBC1_6 sgRNA guide sequence
acctgctctaccccaggcct

SEQ ID NO: 128 - DNA, 20 bp, Artificial Sequence
  TRBC1_7 sgRNA guide sequence
ccaacagtgtcctaccagca

SEQ ID NO: 129 - DNA, 20 bp, Artificial Sequence
  TRBC1_8 sgRNA guide sequence
caacagtgtcctaccagcaa

SEQ ID NO: 130 - DNA, 20 bp, Artificial Sequence
  TRBC1_9 sgRNA guide sequence
aacagtgtcctaccagcaag

SEQ ID NO: 131 - DNA, 20 bp, Artificial Sequence
  TRBC1_10 sgRNA guide sequence
gtctgaaagaaagcagggag

SEQ ID NO: 132 - DNA, 20 bp, Artificial Sequence
  TRBC1_11 sgRNA guide sequence
ccacagtctgaaagaaagca

SEQ ID NO: 133 - DNA, 20 bp, Artificial Sequence
  TRBC1_12 sgRNA guide sequence
gccacagtctgaaagaaagc

SEQ ID NO: 134 - DNA, 20 bp, Artificial Sequence
  TRBC1_13 sgRNA guide sequence
gacactgttggcacggagga

SEQ ID NO: 135 - DNA, 20 bp, Artificial Sequence
  TRBC1_14 sgRNA guide sequence
gtaggacactgttggcacgg

SEQ ID NO: 136 - DNA, 20 bp, Artificial Sequence
  TRBC1_15 sgRNA guide sequence
taccatggccatcaacacaa

SEQ ID NO: 137 - DNA, 20 bp, Artificial Sequence
  TRBC1_16 sgRNA guide sequence
ttaccatggccatcaacaca

SEQ ID NO: 138 - DNA, 20 bp, Artificial Sequence
  TRBC2_1 sgRNA guide sequence
ccagctcagctccacgtggt

SEQ ID NO: 139 - DNA, 20 bp, Artificial Sequence
  TRBC2_2 sgRNA guide sequence
cacagacccgcagcccctca

SEQ ID NO: 140 - DNA, 20 bp, Artificial Sequence
  TRBC2_3 sgRNA guide sequence
gcgggggttctgccagaagg

SEQ ID NO: 141 - DNA, 20 bp, Artificial Sequence
  TRBC2_4 sgRNA guide sequence
gttgcgggggttctgccaga

SEQ ID NO: 142 - DNA, 20 bp, Artificial Sequence
  TRBC2_5 sgRNA guide sequence
atgacgagtggacccaggat

SEQ ID NO: 143 - DNA, 20 bp, Artificial Sequence
  TRBC2_6 sgRNA guide sequence
tgacgagtggacccaggata

SEQ ID NO: 144 - DNA, 20 bp, Artificial Sequence
  TRBC2_7 sgRNA guide sequence
acctgctctaccccaggcct

SEQ ID NO: 145 - DNA, 20 bp, Artificial Sequence
  TRBC2_8 sgRNA guide sequence
tcaacagagtcttaccagca

SEQ ID NO: 146 - DNA, 20 bp, Artificial Sequence
  TRBC2_9 sgRNA guide sequence
caacagagtcttaccagcaa

SEQ ID NO: 147 - DNA, 20 bp, Artificial Sequence
  TRBC2_10 sgRNA guide sequence
aacagagtcttaccagcaag

SEQ ID NO: 148 - DNA, 20 bp, Artificial Sequence
  TRBC2_11 sgRNA guide sequence
cacagtctgaaagaaaacag

SEQ ID NO: 149 - DNA, 20 bp, Artificial Sequence
  TRBC2_12 sgRNA guide sequence
ccacagtctgaaagaaaaca

SEQ ID NO: 150 - DNA, 20 bp, Artificial Sequence
  TRBC2_13 sgRNA guide sequence
gccacagtctgaaagaaaac

SEQ ID NO: 151 - DNA, 20 bp, Artificial Sequence
  PDCD1_1 sgRNA guide sequence
tccaggcatgcagatcccac

SEQ ID NO: 152 - DNA, 20 bp, Artificial Sequence
  PDCD1_2 sgRNA guide sequence
tgcagatcccacaggcgccc

SEQ ID NO: 153 - DNA, 20 bp, Artificial Sequence
  PDCD1_3 sgRNA guide sequence
cgactggccagggcgcctgt

SEQ ID NO: 154 - DNA, 20 bp, Artificial Sequence
  PDCD1_4 sgRNA guide sequence
acgactggccagggcgcctg

SEQ ID NO: 155 - DNA, 20 bp, Artificial Sequence
  PDCD1_5 sgRNA guide sequence
accgcccagacgactggcca

SEQ ID NO: 156 - DNA, 20 bp, Artificial Sequence
  PDCD1_6 sgRNA guide sequence
caccgcccagacgactggcc

SEQ ID NO: 157 - DNA, 20 bp, Artificial Sequence
  PDCD1_7 sgRNA guide sequence
tgtagcaccgcccagacgac

SEQ ID NO: 158 - DNA, 20 bp, Artificial Sequence
  PDCD1_8 sgRNA guide sequence
gggcggtgctacaactgggc

SEQ ID NO: 159 - DNA, 20 bp, Artificial Sequence
  PDCD1_9 sgRNA guide sequence
cggtgctacaactgggctgg

SEQ ID NO: 160 - DNA, 20 bp, Artificial Sequence
  PDCD1_10 sgRNA guide sequence
ctacaactgggctggcggcc

SEQ ID NO: 161 - DNA, 20 bp, Artificial Sequence
  PDCD1_11 sgRNA guide sequence
cacctacctaagaaccatcc

SEQ ID NO: 162 - DNA, 20 bp, Artificial Sequence
  =PDCD1_12 sgRNA guide sequence
ggggttccagggcctgtctg

SEQ ID NO: 163 - DNA, 20 bp, Artificial Sequence
  PDCD1_13 sgRNA guide sequence
gggggttccagggcctgtct

SEQ ID NO: 164 - DNA, 20 bp, Artificial Sequence
  PDCD1_14 sgRNA guide sequence
ggggggttccagggcctgtc

SEQ ID NO: 165 - DNA, 20 bp, Artificial Sequence
  PDCD1_15 sgRNA guide sequence
cagcaaccagacggacaagc

SEQ ID NO: 166 - DNA, 20 bp, Artificial Sequence
  PDCD1_16 sgRNA guide sequence
cccgaggaccgcagccagcc

SEQ ID NO: 167 - DNA, 20 bp, Artificial Sequence
  PDCD1_17 sgRNA guide sequence
ggaccgcagccagcccggcc

SEQ ID NO: 168 - DNA, 20 bp, Artificial Sequence
  PDCD1_18 sgRNA guide sequence
cgtgtcacacaactgcccaa

SEQ ID NO: 169 - DNA, 20 bp, Artificial Sequence
  PDCD1_19 sgRNA guide sequence
gtgtcacacaactgcccaac

SEQ ID NO: 170 - DNA, 20 bp, Artificial Sequence
  PDCD1_20 sgRNA guide sequence
cgcagatcaaagagagcctg

SEQ ID NO: 171 - DNA, 20 bp, Artificial Sequence
  PDCD1_21 sgRNA guide sequence
gcagatcaaagagagcctgc

SEQ ID NO: 172 - DNA, 20 bp, Artificial Sequence
  PDCD1_22 sgRNA guide sequence
agccggccagttccaaaccc

SEQ ID NO: 173 - DNA, 20 bp, Artificial Sequence
  PDCD1_23 sgRNA guide sequence
cggccagttccaaaccctgg

SEQ ID NO: 174 - DNA, 20 bp, Artificial Sequence
  PDCD1_24 sgRNA guide sequence
cagttccaaaccctggtggt

SEQ ID NO: 175 - DNA, 20 bp, Artificial Sequence
  PDCD1_25 sgRNA guide sequence
ggacccagactagcagcacc

SEQ ID NO: 176 - DNA, 20 bp, Artificial Sequence
  PDCD1_26 sgRNA guide sequence
cacctacctaagaaccatcc

SEQ ID NO: 177 - DNA, 20 bp, Artificial Sequence
  PDCD1_27 sgRNA guide sequence
ggagtctgagagatggagag

SEQ ID NO: 178 - DNA, 20 bp, Artificial Sequence
  PDCD1_28 sgRNA guide sequence
tctggaagggcacaaaggtc

SEQ ID NO: 179 - DNA, 20 bp, Artificial Sequence
  PDCD1_29 sgRNA guide sequence
ttctctctggaagggcacaa

SEQ ID NO: 180 - DNA, 20 bp, Artificial Sequence
  PDCD1_30 sgRNA guide sequence
tgacgttacctcgtgcggcc

SEQ ID NO: 181 - DNA, 20 bp, Artificial Sequence
  PDCD1_31 sgRNA guide sequence
tccctgcagagaaacacact

SEQ ID NO: 182 - DNA, 20 bp, Artificial Sequence
  PDCD1_32 sgRNA guide sequence
gagactcaccaggggctggc

SEQ ID NO: 183 - DNA, 20 bp, Artificial Sequence
  PDCD1_33 sgRNA guide sequence
tctttgaggagaaagggaga

SEQ ID NO: 184 - DNA, 20 bp, Artificial Sequence
  PDCD1_34 sgRNA guide sequence
ttctttgaggagaaagggag

SEQ ID NO: 185 - RNA, 99 nt, Artificial Sequence
  1x MS2_3? tracrRNA (F-5)
  misc_feature (77)..(77): x = 2AdP (2-aminopurine)
  misc_feature (97)..(97): U is modified with 2′OMe and phosphorothioate
  misc_feature (98)..(98): U is modified with 2′OMe and phosphorothioate
aacagcauagcaaguuaaaauaaggcuaguccguuaucaacuugaaaaaguggcaccgagucggugcgcggcccggnggaucaccacgggccuuuuuuu

SEQ ID NO: 186 - protein, 1981 aa, Artificial Sequence
  Protein sequence of RNA scaffold mediated recruitment system (2 xUGI)
  MISC_FEATURE (1)..(7): Nuclear Localization Signal (NLS)
  MISC_FEATURE (8)..(235): APOBEC1
  MISC_FEATURE (236)..(261): Linker
  MISC_FEATURE (262)..(378): MCP
  MISC_FEATURE (382)..(399): T2A peptide
  MISC_FEATURE (402)..(408): Nuclear Localization Signal (NLS)
  MISC_FEATURE (409)..(1775): nCAS9D10A
  MISC_FEATURE (1786)..(1868): UGI
  MISC_FEATURE (1879)..(1961): UGI
  MISC_FEATURE (1976)..(1982): Nuclear Localization Signal (NLS)
KKKRKVMSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKELKTPLGDTTHTSPPCPAPELLGGPMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGEGRGSLLTCGDVEENPGPGTPKKKRKVDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

(19) United States
(12) Patent Application Publication
(10) Pub. No.: US 2023/0203502 A1
(43) Pub. Date: Jun. 29, 2023

(54) RNA SCAFFOLDS

(71) Applicants: Horizon Discovery Limited, Cambridge (GB); Dharmacon, Inc., Lafayette, CO (US)

(72) Inventors: MICHAEL RUSHTON, Cambridge (GB); MICHAEL ANBAR, Cambridge (GB); KEVIN HEMPHILL, Lafayette, CO (US)

(21) Appl. No.: 18/008,234

(22) PCT Filed: Jul. 9, 2021
(86) PCT No.: PCT/US2021/041045

§ 371 Date: Dec. 5, 2022

(30) Foreign Application Priority Data
Jul. 10, 2020 (GB) 2010692.8

(51) Int. Cl. C12N 15/115

(52) U.S. Cl. CPC C12N 15/115; C12N 2310/20

(57) ABSTRACT

The present invention discloses an RNA scaffold comprising a tracrRNA; and a recruiting RNA motif with an extension sequence for targeted gene editing and related uses. The method enables precise modifications to be made to the genome whilst minimizing the possibility of off-target effects, making the method particularly suitable for therapeutic applications.

### CROSS REFERENCE TO RELATED APPLICATIONS

This application is a national stage application of PCT/US2021/041045, filed Jul. 9, 2021, which claims the benefit of the filing date of GB 2010692.8, filed Jul. 10, 2020, the entire disclosures of which are incorporated by reference as if set forth fully herein.

### FIELD OF THE INVENTION

The invention relates to RNA scaffolds for CRISPR systems.

### BACKGROUND

CRISPR-Cas technologies are rapidly evolving and the scopes of CRISPR applications are continuously expanding (Lau, The CRISPR Journal, Vol 1, No 6). A key component of a CRISPR system is the guide RNA (gRNA) that forms part of a RNA scaffold that firstly targets the CRISPR system to the desired target in the genome and secondly delivers biologically-active effectors to the target to carry out the desired function. The RNA scaffold must deliver the effectors in precisely the correct orientation and steric conformation to be able to effectively carry out the function, in the specific way to result in a desired output without causing off target effects. Therefore, optimized RNA scaffolds are required for precision genome targeting effector systems.

The present inventors have designed optimised RNA scaffolds for enhanced, targeted performance. The RNA scaffold, system and method provided herein enable precise modifications to be made to the genome whilst minimizing the possibility of off-target effects, making the method and system particularly suitable for therapeutic applications.

### SUMMARY OF THE INVENTION

In a first aspect the invention provides an RNA scaffold comprising:

(a) a tracrRNA; and

(b) an RNA motif with an extension sequence.

In one embodiment, the RNA scaffold according to the first aspect further comprises a crRNA comprising a guide RNA sequence. The RNA scaffold according to the first aspect comprises one or more modification(s). The RNA motif is linked to the 3′ end of the tracrRNA via a linker. In a preferred embodiment, the linker is a single-stranded RNA or a chemical linkage. The single-stranded RNA linker comprises 0-10 nucleotides, preferably 2-6 nucleotides.

In one embodiment, the RNA scaffold according to the first aspect comprises a tracrRNA that is fused to the crRNA comprising a guide RNA sequence forming a single RNA molecule. In other embodiments, the RNA scaffold according to the first aspect comprises the tracrRNA and the crRNA comprising a guide RNA sequence synthesised as separate RNA molecules. In any embodiment, the tracrRNA hybridises to the crRNA via a repeat:anti-repeat region. The tracrRNA comprises the anti-repeat region, the tetra loop and the 3′ constant region of the gRNA when synthesized as a single RNA molecule as shown in FIG. 10B. The tracrRNA comprises the anti-repeat region and the 3′ constant region of the sgRNA when synthesized as separate RNA molecules, and the tetra loop is absent as shown in FIG. 10D. The anti-repeat region of the tracrRNA hybridizes to the repeat region of the crRNA. In a preferred embodiment, the repeat:anti-repeat region is extended.

The RNA scaffold of the present invention comprises one or more RNA motif(s), wherein the one or more RNA motif(s) comprises one or more modification(s). The one or more modification(s) may be at the 5′ end and/or the 3′ end of the one or more RNA motif(s). The RNA scaffold of the present invention may comprise one or more modification(s) including the substitution of the A base at position 10 to 2-aminopurine (2AP). The RNA scaffold may use 2′ deoxy-2-aminopurine or 2′ ribose 2-aminopurine. The RNA scaffold of the present invention may have one or more modification(s) to the backbone and/or sugar moieties of the RNA scaffold. The extension sequence of the RNA motif is a double-stranded extension, wherein the extension sequence of the RNA motif comprises 2-24 nucleotides. In one embodiment, a 4 nucleotide extension results in the stem having 23 nucleotides in total length. In another embodiment, a 10 nucleotide extension results in the stem having 29 nucleotides in total length. In another embodiment, a 16 nucleotide extension results in the stem having 35 nucleotides in total length. In another embodiment, a 26 nucleotide extension results in the stem having 45 nucleotides in total length.

The RNA scaffold of the present invention comprises one or more RNA motif(s) that bind to an aptamer binding molecule. The one or more RNA motif(s) is selected from the following aptamers: MS2, Ku, PP7, SfMu and Sm7. For example, the MS2 aptamer binds to the MCP protein. In a preferred embodiment, the RNA scaffold comprises one recruiting MS2 RNA motif. In other embodiments, the RNA scaffold comprises two recruiting MS2 RNA motifs. In a preferred embodiment, the MS2 aptamer is a wild-type MS2, a mutant MS2, or variants thereof. The mutant MS2 as used herein is a C-5, F-5 hybrid and/or F-5 mutant. The RNA motif of the RNA scaffold according to the present invention recruits an effector module. The effector module as disclosed herein comprises an RNA binding domain capable of binding to the RNA motif and an effector domain. Suitable effector domains are selected from: reporters, tags, molecules, proteins, particulates and nano particles. In a preferred embodiment, the effector domain is a DNA modification enzyme. Suitable DNA modification enzymes are selected from: AID, CDA, APOBEC1, APOBEC3A, APOBEC3B, APOBEC3C, APOBEC3D, APOBEC3F, or other APOBEC family enzymes, ADA, ADAR family enzymes, or tRNA adenosine deaminases.

In a second aspect, the invention provides a system for genetic modification comprising:

(a) a CRISPR protein;

(b) a crRNA of the present invention as defined above;

(c) an RNA scaffold of the present invention as defined above;

(d) an aptamer binding molecule;

(e) an effector module;

The system according to a second aspect comprises components (a)-(e) that are delivered in the form of nucleic acids, protein complexes and/or expressed through any suitable expression vectors.

The system provided herein may comprise a CRISPR protein that is fused to one or more uracil DNA glycosylase (UNG) inhibitor peptide (UGI). In a preferred embodiment, the CRISPR as used in the system according to a second aspect is a Class 2 type II CRISPR protein such as cas9. The CRISPR protein and/or the effector module as used in the system according to the second aspect may comprise one or more nuclear localization signals (NLSs). The CRISPR protein may be a Class 2 Cas protein that is nuclease null or has nickase activity.

The effector module as used in the system according to a second aspect may be an effector fusion protein comprising an RNA binding domain capable of binding to the RNA motif and an effector domain. The system according to the second aspect may use an RNA motif and an effector module comprising an RNA binding domain pair selected from the group consisting of:

a telomerase Ku binding motif and Ku protein or an RNA-binding section thereof,

a telomerase Sm7 binding motif and Sm7 protein or an RNA-binding section thereof,

a MS2 phage operator stem-loop and MS2 coat protein (MCP) or an RNA-binding section thereof,

a PP7 phage operator stem-loop and PP7 coat protein (PCP) or an RNA-binding section thereof,

a SfMu phage Com stem-loop and Com RNA binding protein or an RNA-binding section thereof

In a third aspect, the present invention provides a method for genetically modifying a cell wherein the method comprises introducing into a cell and/or expressing in a cell the system according to the second aspect. The method according to the third aspect may be used to genetically modify a cell including but not limited to correcting a genetic mutation or inactivating the expression of a gene or changing the expression levels of a gene or changing intron-exon splicing. The genetic modification according to the methods provided in the third aspect is a point mutation, optionally wherein the point mutation introduces a premature stop codon, disrupts a start codon, disrupts a splice site or corrects a genetic mutation.

### FIGURES

FIG. 1: FIG. 1A shows the system which comprises of three structural and functional components: (1) a sequence targeting component (e.g., a Cas protein); (2) an RNA scaffold, for sequence recognition and for effector recruitment, that comprises a crRNA, tracrRNA and an RNA motif and (3) an effector module (e.g. a non-nuclease DNA modifying enzyme such as AID fused to a small protein that binds to the RNA motif). More specifically as shown in FIG. 1A, the components of the RNA scaffold mediated recruitment platform include: a sequence targeting component 1 (such as dCas9 or nCas9_{D10A}); an RNA scaffold 2 containing a cRNA comprising a guide RNA (and the repeat of the repeat:anti-repeat stem) for sequence targeting 2.1, a tracrRNA for Cas protein binding 2.2, and an RNA motif for recruiting an effector module 2.3, and an effector module 3 comprising an effector domain 3.1 (e.g., cytidine deaminase) fused to an RNA aptamer ligand 3.2. FIG. 1B shows a schematic of the RNA scaffold mediated recruitment complex at the target sequence: Cas9 (or dCas9 or nCas9) binds to tracrRNA, the RNA motif (e.g. aptamer) recruits the effector module, forming an active RNA scaffold mediated recruitment system capable of editing target residues on the unpaired DNA within the CRISPR R-loop.

FIG. 2: (A) MS2 hairpin sequence with a C-5 substitution and (B) MS2 hairpin sequence containing the F-5 mutant sequence, with the additional substitution of A to d2AP at position A-10 indicated.

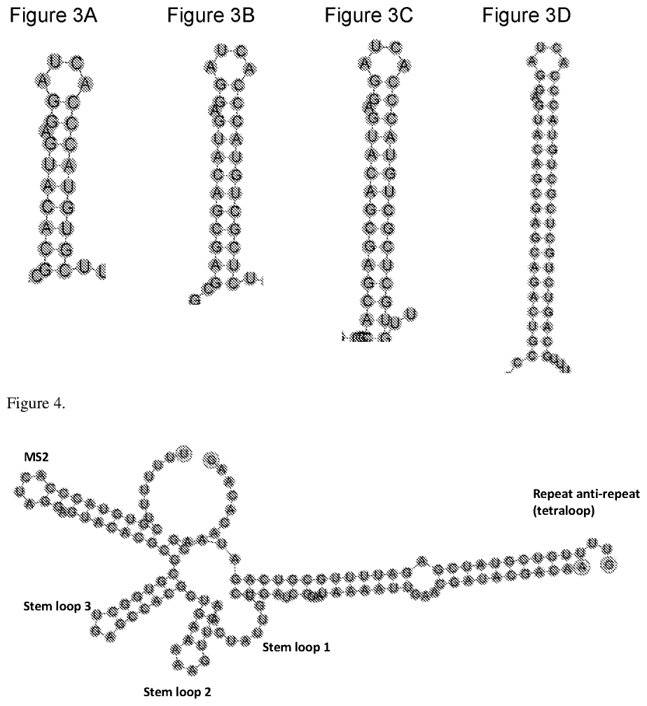

FIG. 3: The RNA motif containing MS2 stem extensions of (A) 4 nt (B) 10 nt (C) 16 nt and (D) (26 nt) relative to the wild type MS2.

FIG. 4: The module of the RNA scaffold comprising a tracrRNA, an RNA motif with an extension sequence, and crRNA comprising a guide RNA sequence.

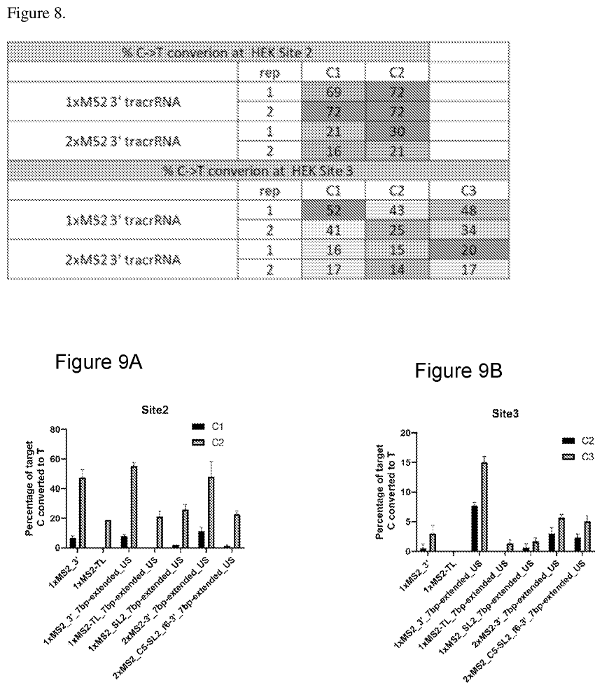

FIG. 5: TRAC Ex3 SA Splice Site Phenotype Disruption Variation Due to Synthetic Aptamers by Cytosine to Thymine Base Changing. Synthetic crRNA:tracrRNA (with and without aptamers) with Electroporated nCas9-UGI-UGI and rApobec1 and hAID Deaminases.

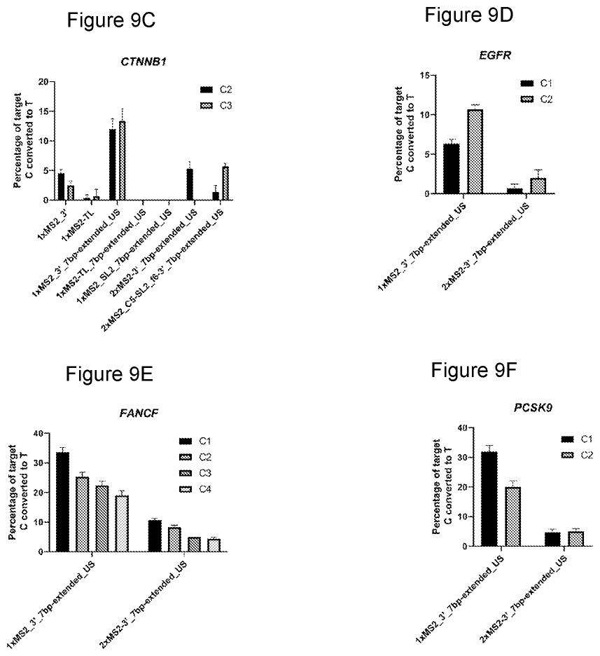

FIG. 6: TRAC Ex3 SA Splice Site Base Change Variation Due to Synthetic Aptamers by Cytosine to Thymine Base Changing. Synthetic crRNA:tracrRNA (with and without aptamers) with electroporated nCas9-UGI-UGI and rApobec1 and hAID Deaminases. Data is shown as the percentage of T sequenced at the indicated target C residue as measured by sanger sequencing.

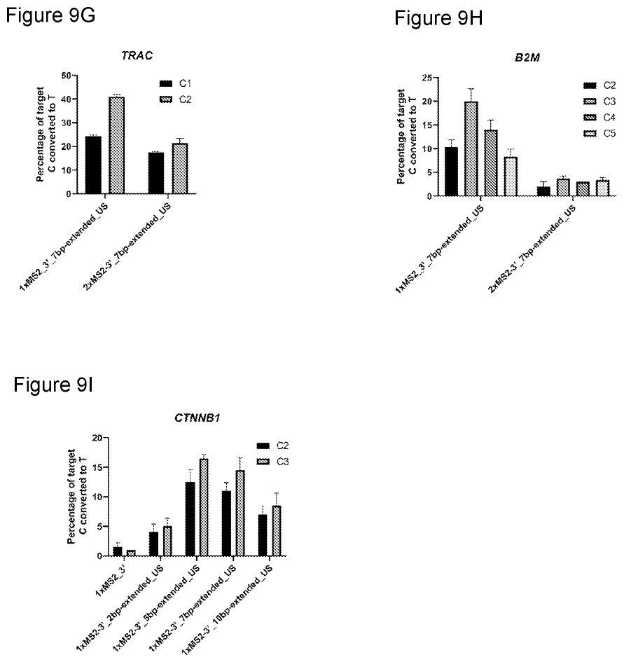

FIG. 7: HEK Site2 editing with tracrRNA containing 4 nt or 16 nt extensions of the MS2 hairpin sequence with nCas9-UGI-UGI and rApobec1 deaminase. Data is shown as the percentage of T sequenced at the indicated target C residue as measured by sanger sequencing.

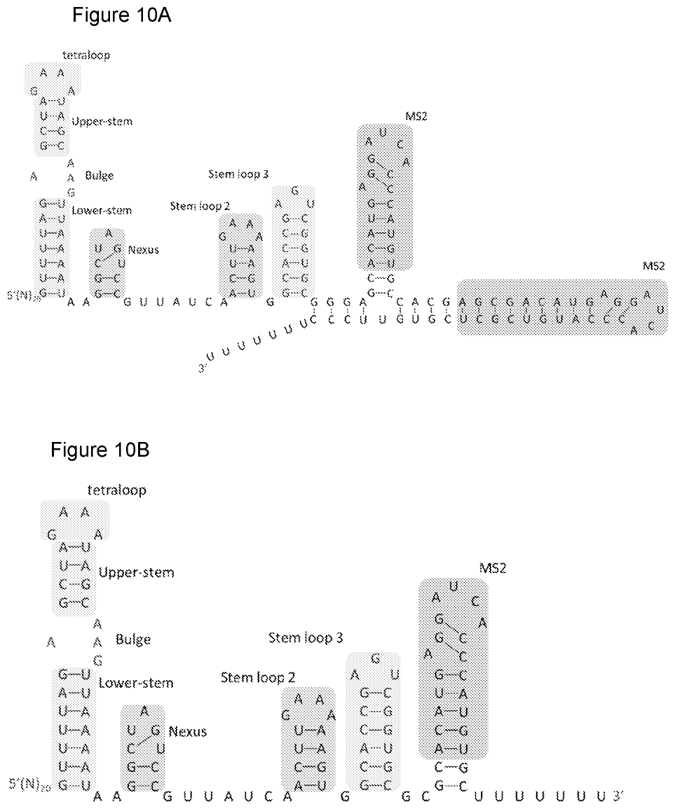

FIG. 8: HEK Site2 and HEK Site3 editing with tracrRNA containing 1 or 2 MS2 hairpins at the 3′ end of the of the RNA motif with nCas9-UGI-UGI and hAID deaminase. Data is shown as the percentage of T sequenced at the indicated target C residue as measured by sanger sequencing.

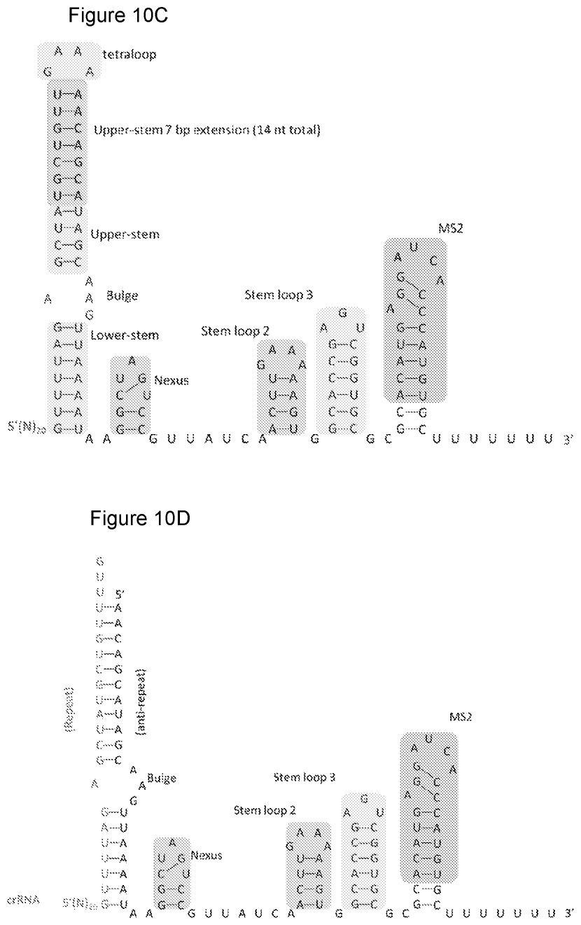

FIG. 9: Base editing efficiencies at different target loci using various RNA scaffold designs. FIG. 9 A-C: The impact of MS2 aptamer location and number, as well as extension of the repeat:anti-repeat upper stem, on APOBEC-1 mediated base editing. Base editing was measured at 3× target loci, the sequence and C residues within the base editing target window are shown in Table 5 of within example 1. The RNA scaffold either incorporated a single copy of the MS2 aptamer (1×MS2) or 2 copies of the MS2 aptamer (2×MS2), and were located either in the tetra-loop (TL), stem-loop 2 (SL2) or the 3′ of the RNA scaffold (3′). Additionally, some designs incorporated a 14-base extension of the repeat:anti-repeat upper stem (7 bp-extended US). Data is shown as the percentage of T sequenced at the indicated target C residue as measured by sanger sequencing. Error bars represent the standard deviation of the mean from 3 replicate experiments. FIG. 9 D-H: Editing by APOBEC-1 was measured at an additional 5 loci, with the previous best 1×MS2_3′ 7 bp-extended US tested along with 2×MS2_3′ 7 bp-extended US. The sequence and C residues within the base editing target window are shown in Table 5 within example 1. Data is shown as the percentage of T sequenced at the indicated target C residue as measured by sanger sequencing. Error bars represent the standard deviation of the mean from 3 replicate experiments. FIG. 9 I: Comparison of the impact of different length extensions of the repeat:anti-repeat upper stem upon aptamer dependent APOBEC-1 mediated base editing. sgRNAs possessing upper-stem extensions of 2 bp, 5 bp, 7 bp and 10 bp as well as a non-extended upper stem (1×MS2_3′) sgRNA were included in the analysis. Data is shown as the percentage of T sequenced at the indicated target C residue as measured by sanger sequencing. Error bars represent the standard deviation of the mean from 3 replicate experiments.

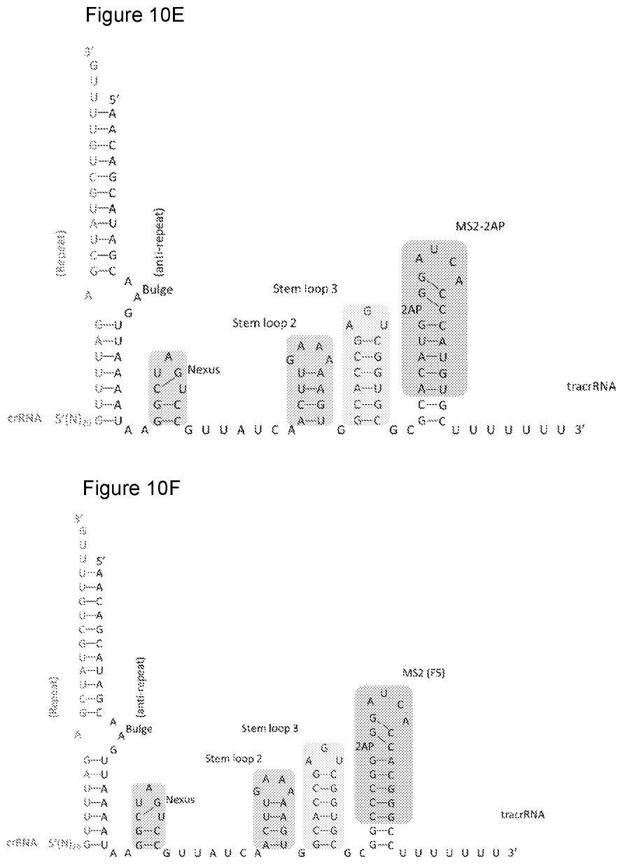

FIG. 10: Annotated diagram illustrating the different portions of the RNA scaffold when synthesised as a single molecule or as separate molecules. FIG. 10A: the RNA scaffold synthesised as a single molecule with two MS2 as disclosed in the prior art WO2017011721. FIG. 10B: the RNA scaffold synthesised as a single molecule with one MS2 as described herein. FIG. 10C: the RNA scaffold synthesised as a single molecule with one MS2 with an extension of 7 bp at either side of the anti-repeat: repeat region. FIG. 10D: the RNA scaffold synthesised as separate molecules wherein the tetra loop is absent. FIG. 10E: the RNA scaffold synthesised as separate molecules with the 2AP modification at position 10 of the MS2 stem loop. FIG. 10F: the RNA scaffold synthesised as separate molecules with the 2AP modification at position 10 of the F-5 mutant of the MS2 stem loop.

FIG. 11: Base editing using chemically synthesized C-5 or F-5 1×MS2_3′ tracrRNA, with crRNA and mRNA of rApobec1deaminase, in nCas9-UGI-UGI U2OS stable cells. Gene sites targeted by each cRNA are (A) CR0118_PDCD1, (B) CR0107_PDCD1, (C) CR0057-TRAC_EX3, (D) CR0151_CD2, (E) HEK Site 2, (F) CR0121_PDCD1, and (G) CR0165_CIITA. Data is shown as the percentage of T sequenced at the indicated target C residue as measured by sanger sequencing.

FIG. 12: Base editing using chemically synthesized C-5 or F-5 1×MS2_3′ tracrRNA, with crRNA and mRNA of hAID deaminase, in nCas9-UGI-UGI U2OS stable cells. Gene sites targeted by each cRNA are (A) CR0151_CD2, (B) CR0121_PDCD1, and (C) CR0165_CIITA. Data is shown as the percentage of T sequenced at the indicated target C residue as measured by sanger sequencing.

FIG. 13: Base editing with chemically synthesized 1×MS2_3′ sgRNAs (C-5), 1×MS2_3′_7 bp-extended_US sgRNAs (C-5) containing a 7-base pair extension of the repeat:anti-repeat upper stem, or 1×MS2_3′ tracrRNA (C-5) with crRNA and mRNA of hAID deaminase in nCas9-UGI-UGI U2OS stable cells. Gene sites targeted by each crRNA are (A) TRAC_22550571, (B) PDCD1_241852953, and (C) CTNNB1. Data is shown as the percentage of T sequenced at the indicated target C residue as measured by sanger sequencing.

FIG. 14. Base editing using chemically synthesized C-5 or F-5 1×MS2_3′ tracrRNA, with crRNA and variable levels of mRNA of rApobec1 deaminase, in nCas9-UGI-UGI U2OS stable cells. Sites targeted by each cRNA are (A) HEK Site 2, (B) CR0107_PDCD1.

### DETAILED DESCRIPTION OF THE INVENTION

The invention relates to new RNA scaffolds for targeting the genome and delivering functional effectors. Such functional effectors include enzymes, reporters, tags, molecules, proteins, particulates, nano particles.

One application of the invention relates to CRISPR gene editing and screening. The invention can be used in any CRISPR gene editing system. An application of the invention involves use of the RNA scaffolds to recruit an effector module to a target DNA sequence in the genome. The invention has particular application in CRISPR base editing systems, for example an RNA scaffold mediated recruitment system.

An example of an RNA scaffold mediated recruitment system comprises the following functional components: (1) a CRISPR/Cas-based module engineered for sequence targeting; (2) an RNA scaffold-based module for guiding the platform to a target sequence as well as for recruitment of an effector module; and (3) a an effector module, such as cytidine deaminases (e.g., activation-induced cytidine deaminase, AID).

In a first aspect, provided herein is an RNA scaffold comprising: (a) a tracrRNA; and (b) an RNA motif with an extension sequence. As disclosed herein, the RNA scaffold are optimised for enhanced gene editing. The RNA scaffold mediated recruitment system is a complex of a number of components including the RNA scaffold that need to be assembled in a specific way to carry out a precise function. The complex has to find a specific part of the genome and arrive in precisely the correct orientation and steric conformation to be able to effectively edit the genome, in the specific way to result in a desired output. Furthermore, the complex has to effectively recruit and deliver a biologically active, effector module such as an enzyme in the correct orientation/configuration to retain enzymatic activity and edit the genome without causing significant off-target effects. Previous base editing systems were associated with poor or limited editing at numerous regions.

In order to overcome these issues, the present inventors have introduced one or more modifications to the RNA scaffold mediated recruitment system, particularly to the RNA scaffold, identified through a trial and error process.

Whilst not wishing to be bound by any theory, it is thought that some of these modifications induce conformational changes to the components of the RNA scaffold mediated recruitment system. A marked improvement was observed with the use of the RNA scaffold as disclosed herein. Advantageously the optimised system comprising an RNA scaffold, itself comprising an RNA motif with an extension sequence, has greater flexibility, stability, positioning and affinity, thereby efficiently editing previously resistant regions including therapeutically relevant loci whilst maintaining performance. The new RNA scaffold expands the repertoire of editable targets and enhances efficiency of gene editing.

RNA Scaffold Mediated Recruitment System

Conventional nuclease-dependent precise genome editing for correction of mutations usually requires introduction of DNA double strand breaks (DSBs) and activation of the homology dependent repair (HDR) pathway.

Recently an RNA-mediated base editing system was also developed. This system recruits a base editing enzyme to a target DNA sequence through the RNA component of a CRISPR complex. This system contains a modified gRNA with a re-programmable RNA-aptamer at the 3′ end, which recruits the cognate aptamer ligand fused to an effector (such as a deaminase effector). Using this system, targeted nucleotide modification was achieved with high precision in prokaryotic cells and eukaryotic cells including mammalian cells; see WO2018129129 and WO2017011721. A new, second generation of RNA-mediated base editing system with increased specificity and efficacy in prokaryotic cells was tested and further improved in mammalian cells. The second generation system/platform exhibits high specificity, high efficiency, and low off-target liability. With a modular design that fully separates the nucleic acid modification module from the nucleic acid recognition module, the RNA-mediated base editing system provides an alternative to recruitment of the effector through fusion to, or direct interaction with, the sequence-targeting protein, which could not effectively separate sequence-targeting function from nucleic acid modification function. The present invention disclosed herein is an RNA scaffold mediated recruitment system which is a modified version of the modular design of the RNA-mediated base editing system. Various modifications have been incorporated to the components of the system thereby improving the flexibility, the specificity and efficiency of said system. The new RNA scaffold mediated recruitment system is not limited to base editing but has a number of possible applications such as genome editing, genome screening and genome tagging, providing a powerful tool for genetic engineering and therapeutic development.

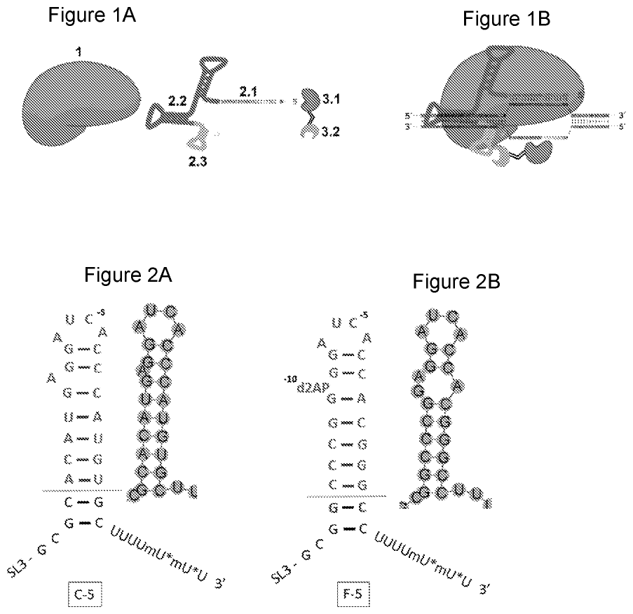

Illustrated in FIGS. 1A and 1B are schematics of an exemplary RNA scaffold mediated recruitment system for use in the methods provided herein. The system includes three structural and functional components: (1) a sequence targeting component (e.g., a Cas protein); (2) an RNA scaffold, for sequence recognition and for effector recruitment, that comprises a crRNA, tracrRNA and an RNA motif and (3) an effector module (e.g. a non-nuclease DNA modifying enzyme such as AID fused to a small protein that binds to the RNA motif). More specifically as shown in FIG. 1A, the components of the RNA scaffold mediated recruitment platform include: a sequence targeting component 1 (such as dCas9 or nCas9_{D10A}); an RNA scaffold 2 containing a cRNA comprising a guide RNA (and the repeat:anti-repeat stem) for sequence targeting 2.1, a tracrRNA for Cas protein binding 2.2, and an RNA motif for recruiting an effector module 2.3, and an effector module 3 comprising an effector domain 3.1 (e.g., cytidine deaminase) fused to an RNA aptamer ligand 3.2. FIG. 1B shows a schematic of the RNA scaffold mediated recruitment complex at the target sequence: Cas9 (or dCas9 or nCas9) binds to tracrRNA, the RNA motif (e.g. aptamer) recruits the effector module, forming an active RNA scaffold mediated recruitment system capable of editing target residues on the unpaired DNA within the CRISPR R-loop. The three components can be constructed in a single expression vector or in multiple separate expression vectors or be introduced in a DNA-free format (mRNA or protein and chemically synthesized RNA molecules). The totality and the combination of the three specific components constitute the enabling of the technologic platform. Although FIG. 1B shows three components of the RNA scaffold in a particular 5′ to 3′ order, the components can also be arranged in different orders when required, such as optimization for different Cas protein variants.

As disclosed herein, there are a number of clear distinctions between recruitment mechanisms: the RNA scaffold mediated recruitment system versus the direct fusion of Cas9 to effector protein system (the BE system). The modular design of the RNA scaffold mediated recruitment system allows for flexible system engineering. Modules are interchangeable and many combinations of different modules can be achieved by simply swapping the nucleotide sequence of the recruiting RNA aptamer and the cognate ligand. Recruitment of an effector by direct fusion or direct interaction with the protein component of the sequence-targeting unit, on the other hand, always requires a re-engineering of a new fusion protein, which is technically more difficult with a less predictable outcome. Furthermore, RNA scaffold mediated recruitment system likely facilitates oligomerization of effector proteins, while direct fusion would preclude the formation of oligomers due to steric hindrance.

Because of its relative ease of use and scalability, the CRISPR/Cas based gene system is poised to dominate the therapeutic landscape, making it an attractive gene editing technology to develop novel applications with therapeutic value. As disclosed herein, the RNA scaffold mediated recruitment system takes advantages of certain aspects of the CRISPR/Cas system. To overcome the limitations associated with requirement of DSB and HDR for conventional CRISPR/Cas gene editing system, an elegant gene editing method called base editing (BE) has been developed exploiting the DNA targeting ability of Cas9 devoid of double-stranded cleavage activity e.g dCas9 or nCas9, combined with the DNA editing capabilities of APOBEC-1, an enzyme member of the APOBEC family of DNA/RNA cytidine deaminases. By directly fusing the deaminase effector to the nuclease deficient Cas9 protein termed dCas9, these tools, called base editors, can introduce targeted point mutations in genomic DNA or RNA without generating DSBs or requiring HDR activity. In essence, the BE system utilizes a nuclease deficient CRISPR/Cas9 complex as a DNA targeting machinery, in which the mutant Cas9 serves as an anchor to recruit cytidine or adenine deaminase through a direct protein-protein fusion.

RNA scaffold mediated recruitment system, on the other hand, takes a different approach. More specifically, in the RNA scaffold mediated recruitment system, the RNA component of the CRISPR/Cas9 complex serves as an anchor for effector recruitment by including an RNA motif such as an aptamer into the RNA molecule. In turn, the RNA aptamer recruits an effector module such as an effector fused to the RNA aptamer ligand. Comparing to the recruitment by direct protein fusion or other recruiting approaches by the protein component, the RNA scaffold mediated recruitment system mechanism has a number of distinct features potentially advantageous both for system engineering and for achieving better functionality. For example, it has a modular design in which the nucleic acid sequence targeting function and effector function reside in different molecules, making it possible to independently reprogram the functional modules and to multiplex the system. The re-programming of the RNA scaffold recruitment mediated system requires only the change of RNA aptamer sequence in gRNA and swap of the cognate RNA aptamer ligand fusing effector. It does not require re-engineering of an individual functional Cas9 fusion protein. In addition, the effector module is smaller in size which could potentially allow more efficient oligomerization of the functional effector. Moreover, as the RNA scaffold mediated recruitment does not require generation of a Cas9 fusion protein, which further increases the gene/transcription size of Cas9, the system could potentially be constructed in a way that is more efficient for packaging and delivery by viral vectors, non-viral vectors, mRNA molecules, mechanical means, or protein components.

As disclosed herein, this invention provides further engineering of the RNA scaffold mediated recruitment system for precision gene editing. As demonstrated herein, the optimised RNA scaffold recruitment system exhibits a number of important different features compared to the previous RNA mediated base editing system described in WO2018129129 and WO2017011721 (incorporated herein by reference in their entirety). First, the optimised RNA scaffold recruitment system exhibit substantially increased on-target efficacy compared to the first generation and second generation RNA mediated base editing system, but still maintains low or absent detectable off-target effect. Second, the optimised RNA scaffold recruitment system has greater flexibility due to modifications incorporated into the various components of the system e.g. the extension sequence at the 3′ end of the RNA motif. Third, the optimised RNA scaffold has improved steric hindrance due to the positioning of the RNA motif in relation to the tracrRNA.

a. Sequence-Targeting Module

The sequence-targeting component of the methods and systems provided herein typically utilise a Cas protein of CRISPR/Cas systems from bacterial species as the sequence targeting protein.

In embodiments the Cas protein is mutant Cas protein, for example, a dCas protein which contains mutations at its nuclease catalytic domains thus does not have nuclease activity, or a nCas protein which is partially mutated at one of the catalytic domains thus does not have nuclease activity for generating DSB. The Cas protein is specifically recognized by the tracrRNA component of the RNA scaffold, which guides the Cas protein to its target DNA or RNA sequence. The latter is flanked by a 3′ PAM.

Cas Proteins

Various Cas proteins can be used in this invention. A Cas protein, CRISPR-associated protein, or CRISPR protein, used interchangeably, refers to a protein of or derived from a CRISPR-Cas Class 1 or Class 2 system, which has an RNA-guided DNA-binding. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. e.g. Koonin and Makarova, 2019, origins and evolution of crispr cas systems, Review Philos Trans R Soc Lond B Biol Sci. 2019 May 13; 374(1772).

In one embodiment, the Cas protein is derived from a Class 2 CRISPR-Cas system. In a preferred embodiment, the Cas protein is a class 2 type 2 cas system. In exemplary embodiments, the Cas protein is or is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus Acaryochloris marina, Legionella pneumophila, Francisella novicida, gamma proteobacterium HTCC5015, Parasutterella excrementihominis, Sutter ella wadsworthensis, Sulfurospirillum sp. SC ADC, Ruminobacter sp. RM87, Burkholderiales bacterium 1 1 47, Bacteroidetes oral taxon 274 str. F0058, Wolinella succinogenes, Burkholderiales bacterium YL45, Ruminobacter amylophilus, Campylobacter sp. P0111, Campylobacter sp. RM9261, Campylobacter lanienae strain RM8001, Camplylobacter lanienae strain P0121, Turicimonas muris, Legionella londiniensis, Salinivibrio sharmensis, Leptospira sp. isolate FW.030, Moritella sp. isolate NORP46, Fndozoicomonassp. S-B4-1 U, Tamilnaduibacter salinus, Vibrio natriegens, Arcobacter skirrowii, Francisella philomiragia, Francisella hispaniensis, or Parendozoicomonas haliclonae.

In general, a Cas protein includes at least one RNA binding domain. The RNA binding domain interacts with the guide RNA. The Cas protein can be a wild type Cas protein or a modified version with no nuclease activity or just single-strand nicking activity. The Cas protein can be modified to increase nucleic acid binding affinity and/or specificity, alter an enzymatic activity, and/or change another property of the protein. For example, nuclease (i.e., DNase, RNase) domains of the protein can be modified, deleted, or inactivated. Alternatively, the protein can be truncated to remove domains that are not essential for the function of the protein. The protein can also be truncated or modified to optimize the activity.

In some embodiments, the Cas protein can be a mutant of a wild type Cas protein (such as Cas9) or a fragment thereof. In other embodiments, the Cas protein can be derived from a mutant Cas protein. For example, the amino acid sequence of the Cas9 protein can be modified to alter one or more properties (e.g., nuclease activity, affinity, stability, etc.) of the protein. Alternatively, domains of the Cas9 protein not involved in RNA targeting can be eliminated from the protein such that the modified Cas9 protein is smaller than the wild type Cas9 protein. In some embodiments, the present system utilizes the Cas9 protein from S. pyogenes, either as encoded in bacteria or codon-optimized for expression in mammalian cells.

A mutant Cas protein refers to a polypeptide derivative of the wild type protein, e.g., a protein having one or more point mutations, insertions, deletions, truncations, a fusion protein, or a combination thereof. The mutant has at least one of the RNA-guided DNA binding activity, or RNA-guided nuclease activity, or both. In general, the modified version is at least 50% (e.g., any number between 50% and 100%, inclusive, e.g., 50%, 60%, 70%, 75%, 80%, 85%, 90%, 95%, and 99%) identical to the wild type protein such as SEQ ID NO: 1.

A Cas protein (as well as other protein components described in this invention) can be obtained as a recombinant polypeptide. To prepare a recombinant polypeptide, a nucleic acid encoding it can be linked to another nucleic acid encoding a fusion partner, e.g., glutathione-s-transferase (GST), 6×-His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this invention. Alternatively, the proteins can be chemically synthesized using routine methods known in the art or produced by recombinant DNA technology as described herein and using methods known in the art.

The Cas protein described in the invention can be provided in purified or isolated form, or can be part of a composition. Preferably, where in a composition, the proteins are first purified to some extent, more preferably to a high level of purity (e.g., about 80%, 90%, 95%, or 99% or higher). Compositions according to the invention can be any type of composition desired, but typically are aqueous compositions suitable for use as, or inclusion in, a composition for RNA-guided targeting. Those of skill in the art are well aware of the various substances that can be included in such nuclease reaction compositions.

To practice the method disclosed herein for modifying a target nucleic acid, one can produce the proteins in a target cell via mRNA, protein RNA complexes (RNP), or any suitable expression vectors. Examples of expression vectors include chromosomal, non-chromosomal and synthetic DNA sequences, bacterial plasmids, minicircles, phage DNA, baculovirus, yeast plasmids, vectors derived from combinations of plasmids and phage DNA, viral DNA such as vaccinia, adenovirus, fowl pox virus, and pseudorabies. More details are described in the Expression System and Methods sections below.

As disclosed here, one can use the nuclease dead Cas9 (dCas9, for example from S. pyogenes D10A, H840A mutant protein), or the nuclease defective nickase Cas9 (nCas9, for example from S. pyogenes D10A mutant protein). dCas9 or nCas9 could also be derived from various bacterial species. Table 1 lists a non-exhausting list of examples of Cas9, and their corresponding PAM requirements. One can also use synthetic Cas substitutes such as those described in Rauch et al., Programmable RNA-Guided RNA Effector Proteins Built from Human Parts. Cell Volume 178, Issue 1, 27 Jun. 2019, Pages 122-134.e12.

TABLE 1

| Species | 5′ to 3′ PAM |
|---|---|
| Streptococcus pyogenes | NGG |
| Streptococcus agalactiae | NGG |
| Staphylococcus aureus | NNGRRT |
| Streptococcus thermophiles | NNAGAAW |
| Streptococcus thermophiles | NGGNG |
| Neisseria meningitidis | NNNNGATT |
| Treponema denticola | NAAAAC |
| Other Type II CRISPR/Cas9 | |
| systems from other bacterial | |
| species | |

N is any nucleotides (A or G or T or C), R is A or G, and W is A or T.

UGI

In some embodiments of this disclosure, the above-described sequence-targeting component comprises a fusion between (a) a CRISPR protein, and (b) a first uracil DNA glycosylase (UNG) inhibitor peptide (UGI). For example, the fusion protein can include a Cas protein, e.g. Cas9 protein, fused to a UGI. Such fusion proteins may exhibit an increased nucleic acid editing efficiency as compared to fusion proteins not comprising an UGI domain. In some embodiments, the UGI comprises a wild type UGI sequence or one having the following amino acid sequence: sp|P14739|UNGI_BPPB2: Uracil-DNA glycosylase inhibitor (UGI)

SEQ ID NO: 2
MTNLSDIIEKETGKQLVIQESILMLPEEVEEVIGNKPESDILVHTAYDE
STDENVMLLTSDAPEYKPWALVIQDSNGENKIKML.

In some embodiments, the UGI proteins provided herein include fragments of UGI and proteins homologous to a UGI or a UGI fragment. For example, in some embodiments, a UGI comprises a fragment of the amino acid sequence set forth above. In some embodiments, a UGI comprises an amino acid sequence homologous to the amino acid sequence set forth above or an amino acid sequence homologous to a fragment of the amino acid sequence set forth in the UGI sequence above. In some embodiments, proteins comprising UGI or fragments of UGI or homologs of UGI or UGI fragments are referred to as “UGI variants.” A UGI variant shares homology to UGI, or a fragment thereof. For example, a UGI variant is at least about 70% (e.g., at least about 80%, 90%, 95%, 96%, 97%, 98%, 99%) to a wild type UGI or the UGI sequence as set forth above.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J Mol. Biol. 287:331-346(1999), the entire contents of each are incorporated herein by reference.

b. RNA Scaffold for Sequence Recognition and Effector Recruitment:

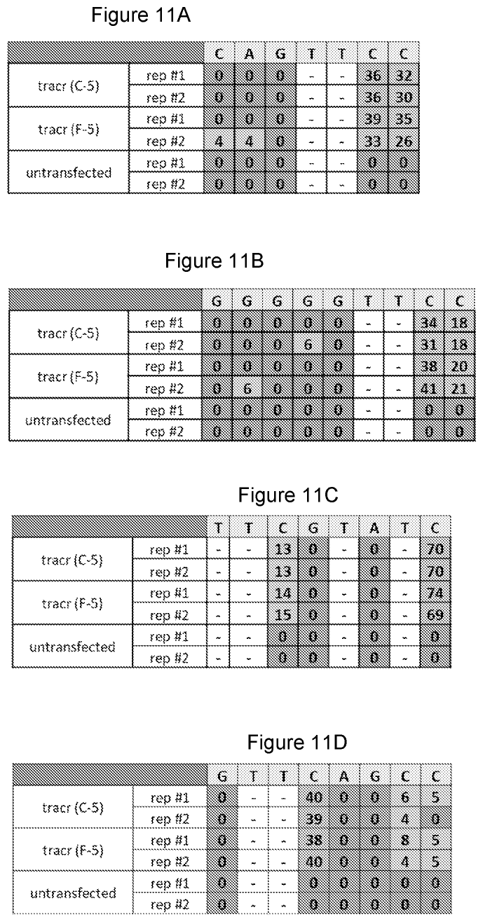

The second component of the platform disclosed herein is an RNA scaffold, which has three sub-components: a crRNA comprising a guide RNA sequence, a trans-activating CRISPR RNA (tracrRNA), and an RNA motif with an extension sequence. This scaffold can be either a single RNA molecule or a complex of multiple RNA molecules. The crRNA comprising a guide RNA of the RNA scaffold is linked to the tracrRNA through a repeat:anti-repeat region which consists of a 7-bp lower stem and a 4 bp upper stem interspersed by a 4 nucleotide bulge structure. When the RNA scaffold is expressed as a single molecule the repeat:anti-repeat region is connected by a tetra-loop comprising 4 nucleotides as shown in FIG. 10B. When the RNA scaffold is expressed as multiple RNA molecules, the tetra-loop is absent and the repeat:anti-repeat region links the crRNA and tracrRNA RNA molecules as shown in FIG. 10D.

As disclosed herein, the crRNA comprising a programmable guide RNA, tracrRNA and the Cas protein together form a CRISPR/Cas-based module for sequence targeting and recognition, while the RNA motif recruits, via an RNA-protein binding pair, an effector module, such as a base editing enzyme, which carries out the genetic modification. Accordingly, the RNA scaffold connects the effector module (e.g. base editing enzyme) and sequence recognition module (e.g. Type II Cas protein). The RNA scaffold as disclosed herein comprises one or more modifications.

Programmable Guide RNA (crRNA)

One key sub-component is the programmable guide RNA. Due to its simplicity and efficiency, the CRISPR-Cas system has been used to perform genome-editing in cells of various organisms. The specificity of this system is dictated by base pairing between a target DNA and a custom-designed guide RNA. By engineering and adjusting the base-pairing properties of guide RNAs, one can target any sequences of interest provided that there is a PAM sequence adjacent to the target sequence.

Among the sub-components of the RNA scaffold disclosed herein, the guide sequence provides the targeting specificity. It includes a region that is complementary and capable of hybridization to a pre-selected target site of interest. In various embodiments, the target specifying component of the guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In an exemplary embodiment, the guide sequence is about 17-20 nucleotides in length, such as 20 nucleotides.

Additionally the crRNA possesses a constant region 3′ of the target specifying sequence. This sequence forms the repeat:anti-repeat stem that links the crRNA to the tracrRNA components of the RNA scaffold. The constant 3′ sequence of the crRNA is complementary to the 5′ sequence of the tracrRNA and as such forms a duplex stem. The repeat:anti-repeat region of the RNA scaffold can be split into 3 parts; the lower stem, bulge and upper stem. The lower stem is 7 bp in length form through both Watson-Crick and non-Watson-Crick base pairing; this is followed by a bulge structure of 4 nucleotides. The upper stem consists of a 4 bp structure. When synthesized as a single RNA molecule, the tracrRNA comprises the anti-repeat region, the tetraloop, and the 3′ constant region of the sgRNA. When synthesized as a separate RNA molecule, the tracrRNA comprises the anti-repeat region and the 3′ constant region of the sgRNA, but the tetraloop is absent.

One requirement for selecting a suitable target nucleic acid is that it has a 3′ PAM site/sequence. Each target sequence and its corresponding PAM site/sequence are referred herein as a Cas-targeted site. The Class 2 CRISPR system such as the type II enzymes, one of the most well characterized systems, needs only Cas9 protein and a guide RNA complementary to a target sequence to affect target cleavage. The Class 2 type II CRISPR system of S. pyogenes such as cas9 uses target sites having N12-20NGG, where NGG represents the PAM site from S. pyogenes, and N12-20 represents the 12-20 nucleotides directly 5′ to the PAM site. Additional PAM site sequences from other species of bacteria include NGGNG, NNNNGATT, NNAGAA, NNAGAAW, and NAAAAC. See, e.g., US 20140273233, WO 2013176772, Cong et al., (2012), Science 339 (6121): 819-823, Jinek et al., (2012), Science 337 (6096): 816-821, Mali et al, (2013), Science 339 (6121): 823-826, Gasiunas et al., (2012), Proc Natl Acad Sci USA. 109 (39): E2579-E2586, Cho et al., (2013) Nature Biotechnology 31, 230-232, Hou et al., Proc Natl Acad Sci USA. 2013 Sep. 24; 110(39):15644-9, Mojica et al., Microbiology. 2009 March; 155(Pt 3):733-40, and www.addgene.org/CRISPR/. The contents of these documents are incorporated herein by reference in their entireties.

The target nucleic acid strand can be either of the two strands on a genomic DNA in a host cell. Examples of such genomic dsDNA include, but are not necessarily limited to, a host cell chromosome, mitochondrial DNA and a stably maintained plasmid. However, it is to be understood that the present method can be practiced on other dsDNA present in a host cell, such as non-stable plasmid DNA, viral DNA, and phagemid DNA, as long as there is Cas-targeted site regardless of the nature of the host cell dsDNA. The present method can be practiced on RNAs too.

tracrRNA

Besides the above-described guide sequence, the RNA scaffold of this invention includes additional active or non-active sub-components. In one example, the scaffold has a tracrRNA. For example, the scaffold can be a hybrid RNA molecule where the above-described crRNA comprising a programmable guide RNA is fused to a tracrRNA to mimic the natural crRNA:tracrRNA duplex. Shown below is an exemplary hybrid crRNA:tracrRNA, gRNA sequence SEQ ID NO:3: 5′-(20 nt guide)-GUUUAAGAGCUAUGCUGGAAACAGCAUAGCAAGUUUAAAUAAGGCUAGUCCGUUAUCA ACUUGAAAAAGUGGCACCGAGUCGGUGCUUUUUUU-3′. Various tracrRNA sequences are known in the art and examples include the following tracrRNAs and active portions thereof. As used herein, an active portion of a tracrRNA retains the ability to form a complex with a Cas protein, such as Cas9 or dCas9 or nCas9. Methods for generating crRNA-tracrRNA hybrid RNAs (also known as single guide RNAs or sgRNAs) are known in the art. In one embodiment where the crRNA and tracrRNA are provided as a single gRNA (sgRNA), the two components be linked together via a tetra stem loop. In some embodiments, the repeat anti-repeat region is extended. There is an extension of 2, 3, 4, 5, 6, 7 bases or more than 7 bases at either side of the repeat:anti-repeat region. In a preferred embodiment, the repeat: anti-repeat region has an extension of 7 nucleotides at either side of the upper stem as shown in FIG. 10C and FIG. 10D. The extension of 7 bases at either side of the upper stem results in a region that is 14 base pairs longer. The 7 base extension at either side of the upper stem results in the upper stem having a total of 11 bases at either side and a total length of 22 nucleotides when the RNA scaffold is synthesized as one single RNA molecule as shown in FIG. 10C. The 7 base extension at either side of the upper stem results in the upper stem having a total of 11 bases at either side and a total length of 25 nucleotides when the RNA scaffold is synthesized as two separate RNA molecule as shown in FIG. 10D. In one embodiment, the total length of the upper stem of the repeat:anti-repeat region is 22 nucleotides when the RNA scaffold is synthesised as a single RNA molecule. In other embodiments, the total length of the upper stem of the repeat:anti-repeat region is 25 nucleotides when the RNA scaffold is synthesised as two separate RNA molecules. In other embodiments, the extension may be more than 7 bases.

See e.g., WO2014099750, US 20140179006, and US 20140273226. The contents of these documents are incorporated herein by reference in their entireties.

TracrRNA sequences for S. pyogenes Cas9 with various truncations and extensions are shown below:

(SEQ ID NO: 4)
GGAACCAUUCAAAACAGCAUAGCAAGUUAAAAUAAGGCUAGUCCGUUAU
CAACUUGAAAAAGUGGCACCGAGUCGGUGC;
(SEQ ID NO: 5)
UAGCAAGUUAAAAUAAGGCUAGUCCGUUAUCAACUUGAAAAAGUGGCAC
CGAGUCGGUGC;
(SEQ ID NO: 6)
AGCAUAGCAAGUUAAAAUAAGGCUAGUCCGUUAUCAACUUGAAAAAGUG
GCACCGAGUCGGUGC;
(SEQ ID NO: 7)
CAAAACAGCAUAGCAAGUUAAAAUAAGGCUAGUCCGUUAUCAACUUGAA
AAAGUGGCACCGAGUCGGUGC;
(SEQ ID NO: 8)
UAGCAAGUUAAAAUAAGGCUAGUCCGUUAUCAACUUGAAAAAGUG;
(SEQ ID NO: 9)
UAGCAAGUUAAAAUAAGGCUAGUCCGUUAUCA;
and
(SEQ ID NO: 10)
UAGCAAGUUAAAAUAAGGCUAGUCCG.

In some embodiments the tracrRNA is from Strep pyogenes.

In some embodiments, the tracrRNA and the crRNA comprising the guide sequence are two separate RNA molecules, which together form the functional guide RNA and part of the RNA scaffold.

In this case, the tracrRNA should be able to interact with (usually by base pairing) the crRNA having the guide sequence to form a two part guide crRNA:tracRNA.

RNA Motif

The third sub-component of the RNA scaffold is the RNA motif(s), which, in effect, recruits the effector module (base editing enzyme) to the target DNA. The RNA motif is also referred to as the recruiting RNA motif. This linkage is critical for the gene editing systems and methods disclosed herein. The RNA scaffold as disclosed herein may have one or more RNA motif(s).

A prior art method to recruit effector/DNA editing enzymes to a target sequence is through a direct fusion of an effector protein to dCas9. The direct fusion of effector enzymes to the proteins required for sequence recognition (such as dCas9) has achieved success in sequence specific transcriptional activation or suppression, but the protein-protein fusion design may render spatial hindrance, which is not ideal for enzymes that need to form a multimeric complex for their activities. In fact, most nucleotide editing enzymes (such as AID or APOBEC3G) require formation of dimers, tetramers or higher order oligomers, for their DNA editing catalytic activities. The direct fusion to dCas9, which anchors to DNA in a defined conformation, would hinder the formation of a functional oligomeric enzyme complex at the right location.

In contrast, the RNA scaffold mediated recruitment system and methods provided herein are based on RNA scaffold-mediated effector protein recruitment. More specifically, the platform takes advantage of various RNA motif/RNA binding protein binding pairs. To this end, an RNA scaffold is designed such that an RNA motif (e.g., MS2 operator motif), which specifically binds to an aptamer binding molecule such as an RNA binding protein (e.g., MS2 coat protein, MCP), is linked to the RNA scaffold via a linker sequence at the 3′ end of the tracrRNA. The linker may be a single-stranded RNA or a chemical linkage. In one embodiment, a single-stranded linker comprises 0-10 nucleotides, preferably 2-6 nucleotides. The single stranded sequence may comprise GC nucleotides. Advantageously, the linker e.g. the single stranded linker separates the loop of the RNA motif from the bulky stem loop of the tracrRNA. The one or more RNA motif(s) as disclosed herein has an extension sequence. In a preferred embodiment, the extension sequence is a double-stranded extension. The extension sequence varies in length comprising of 2-24 nucleotides. In some embodiments, the one or more RNA motif(s) comprises one or more modifications. The one or more modifications may be at the 5′ end and/or the 3′ end of the one or more RNA motif(s).

As a result, this RNA scaffold component of the platform disclosed herein is a designed RNA molecule, which contains not only the crRNA for specific DNA/RNA sequence recognition, the tracrRNA for Cas protein binding, but also the RNA motif for effector recruitment (FIG. 1B). In this way, recruited-effector modules can be recruited to the target site through their ability to bind to the RNA motif. Due to the flexibility of RNA scaffold mediated recruitment, a functional monomer, as well as dimer, tetramer, or oligomer could be relatively easy to form near the target DNA or RNA sequence. These pairs of RNA motif/binding protein could be derived from naturally occurring sources (e.g., RNA phages, or yeast telomerase) or could be artificially designed (e.g., RNA aptamers and their corresponding binding protein ligands). A non-exhaustive list of examples of recruiting RNA motif/RNA binding protein pairs that could be used in the methods and systems provided herein is summarized in Table 2.

TABLE 2
Examples of recruiting RNA motifs that can be used in this invention,
as well as their paring RNA binding proteins/protein domains.

| RNA motif | Pairing interacting protein* | Organism |
|---|---|---|
| Telomerase Ku binding motif | Ku | Yeast |
| Telomerase Sm7 binding motif | Sm7 | Yeast |
| MS2 phage operator stem- | MS2 Coat Protein (MCP) | Phage |
| loop | | Phage |
| PP7 phage operator stem-loop | PP7 coat protein (PCP) | Phage |
| SfMu phage Com stem-loop | Com RNA binding protein | Artificially |
| Non-natural RNA aptamer | Corresponding aptamer | designed |
| | ligand | |

*Recruited proteins are fused to effector proteins, for examples see Table 3.

The sequences for the above binding pairs are listed below.

1. Telomerase Ku biding motif/Ku heterodimer
a. Ku binding hairpin
5′-
UUCUUGUCGUACUUAUAGAUCGCUACGUUAUUUCAAUUUUGAAAAUCUGAGUCCUGGGAGUGCG
GA-3′ SEQ ID NO: 11
Ku heterodimer SEQ ID NO: 12
MSGWESYYKTEGDEEAEEEQEENLEASGDYKYSGRDSLIFLVDASKAMFESQSEDELTPFDMSI
QCIQSVYISKIISSDRDLLAVVFYGTEKDKNSVNFKNIYVLQELDNPGAKRILELDQFKGQQGQ
KRFQDMMGHGSDYSLSEVLWVCANLFSDVQFKMSHKRIMLFTNEDNPHGNDSAKASRARTKAGD
LRDTGIFLDLMHLKKPGGFDISLFYRDIISIAEDEDLRVHFEESSKLEDLLRKVRAKETRKRAL
SRLKLKLNKDIVISVGIYNLVQKALKPPPIKLYRETNEPVKTKTRTFNTSTGGLLLPSDTKRSQ
IYGSRQIILEKEETEELKRFDDPGLMLMGFKPLVLLKKHHYLRPSLFVYPEESLVIGSSTLFSA
LLIKCLEKEVAALCRYTPRRNIPPYFVALVPQEEELDDQKIQVTPPGFQLVFLPFADDKRKMPF
TEKIMATPEQVGKMKAIVEKLRFTYRSDSFENPVLQQHFRNLEALALDLMEPEQAVDLTLPKVE
AMNKRLGSLVDEFKELVYPPDYNPEGKVTKRKHDNEGSGSKRPKVEYSEEELKTHISKGTLGKF
TVPMLKEACRAYGLKSGLKKQELLEALTKHFQD>
MVRSGNKAAVVLCMDVGFTMSNSIPGIESPFEQAKKVITMFVQRQVFAENKDEIALVLFGTDGT
DNPLSGGDQYQNITVHRHLMLPDFDLLEDIESKIQPGSQQADFLDALIVSMDVIQHETIGKKFE
KRHIEIFTDLSSRFSKSQLDIIIHSLKKCDISERHSIHWPCRLTIGSNLSIRIAAYKSILQERV
KKTWTVVDAKTLKKEDIQKETVYCLNDDDETEVLKEDIIQGFRYGSDIVPFSKVDEEQMKYKSE
GKCFSVLGFCKSSQVQRRFFMGNQVLKVFAARDDEAAAVALSSLIHALDDLDMVAIVRYAYDKR
ANPQVGVAFPHIKHNYECLVYVQLPFMEDLRQYMFSSLKNSKKYAPTEAQLNAVDALIDSMSLA
KKDEKTDTLEDLFPTTKIPNPRFQRLFQCLLHRALHPREPLPPIQQHIWNMLNPPAEVTTKSQI
PLSKIKTLFPLIEAKKKDQVTAQEIFQDNHEDGPTAK
‘>’ separates the two dimers.
2. Telomerase Sm7 biding motif/Sm7 homoheptamer
a. Sm consensus site (single stranded)
5′-AAUUUUUGGA-3′ SEQ ID NO: 13
Monomeric Sm-like protein (archaea) SEQ ID NO: 14
GSVIDVSSQRVNVQRPLDALGNSLNSPVIIKLKGDREFRGVLKSFDLHMNLVLNDAEELEDGEV
TRRLGTVLIRGDNIVYISP
3. MS2 phage operator stem loop/MS2 coat protein
a. MS2 phage operator stem loop
5′-ACAUGAGGAUCACCCAUGU-3′ SEQ ID NO: 15
MS2 coat protein SEQ ID NO: 16
MASNFTQFVLVDNGGTGDVTVAPSNFANGIAEWISSNSRSQAYKVTCSVRQSSAQNRKYTIKVE
VPKGAWRSYLNMELTIPIFATNSDCELIVKAMQGLLKDGNPIPSAIAANSGIY
4. PP7 phage operator stem loop/PP7 coat protein
a. PP7 phage operator stem loop
5′-aUAAGGAGUUUAUAUGGAAACCCUUA-3′ SEQ ID NO: 17
PP7 coat protein (PCP) SEQ ID NO: 18
MSKTIVLSVGEATRTLTEIQSTADRQIFEEKVGPLVGRLRLTASLRQNGAKTAYRVNLKLDQAD
VVDCSTSVCGELPKVRYTQVWSHDVTIVANSTEASRKSLYDLTKSLVATSQVEDLVVNLVPLGR
5. SfMu Com stem loop/SfMu Com binding protein
a. SfMu Com stem loop
5′-CUGAAUGCCUGCGAGCAUC-3′ SEQ ID NO: 19
a. SfMu Com binding protein SEQ ID NO: 20
MKSIRCKNCNKLLFKADSFDHIEIRCPRCKRHIIMLNACEHPTEKHCGKREKITHSDETVRY

The RNA scaffold can be either a single RNA molecule or a complex of multiple RNA molecules. For example, the guide RNA, tracrRNA, and RNA motif(s) can be three segments of one, long single RNA molecule. Alternatively, one, two or three of them can be on separate molecules. In the latter case, the three components can be linked together to form the scaffold via covalent or non-covalent linkage or binding, including e.g., Watson-Crick base-pairing.

In one example, the RNA scaffold can comprise two separate RNA molecules. The first RNA molecule can comprise the crRNA comprising a programmable guide RNA and a region that can form a stem duplex structure with a complementary region. The second RNA molecule can comprise the complementary region in addition to the tracrRNA and the RNA motif(s). Via this stem duplex structure, the first and second RNA molecules form an RNA scaffold of this invention. In one embodiment, the first and second RNA molecules each comprise a sequence (of about 6 to about 20 nucleotides) that base pairs to the other sequence. By the same token, the tracrRNA and the RNA motif can also be on different RNA molecule and be brought together with another stem duplex structure.

The RNAs and related scaffold of this invention can be made by various methods known in the art including cell-based expression, in vitro transcription, and chemical synthesis, or combinations thereof. The ability to chemically synthesize relatively long RNAs (as long as 200 mers or more) allows one to produce RNAs with special features that outperform those enabled by the basic four ribonucleotides (A, C, G and U).

The Cas protein-guide RNA scaffold complexes can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties. The complexes can be isolated or purified, at least to some extent, from cellular material of a cell or an in vitro translation-transcription system in which they are produced.

Modifications

The RNA scaffold as disclosed herein may include one or more modifications.

Such modifications may include inclusion and/or removal of at least one non-naturally occurring nucleotide, or a modified nucleotide, or analogues thereof. Examples of such modifications include, but are not limited to the addition of nucleotides to extend sequences, substitution of nucleotides, addition of linker sequences, removal of nucleotides and modifying the positioning of various components of the RNA scaffold. One or more modification(s) is to the backbone and/or sugar moieties of the RNA scaffold.

Nucleotides may be modified at the ribose, phosphate linkage, and/or base moiety. Modified nucleotides may include 2′-O-methyl analogs, 2′-fluoro analogs or 2′-deoxy analogs or 2′-ribose analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, 5-methylcytidine, 5-methoxyuridine, pseudouridine, inosine, 7-methylguanosine. These modifications may apply to any component of the RNA scaffold. These modifications may apply to any component of the CRISPR system. In a preferred embodiment these modifications are made to the RNA components, e.g., the guide RNA sequence.

In some embodiments, the RNA scaffold described above or a subsection thereof can comprise one or more modifications, e.g., a base modification, a backbone modification, etc, to provide the nucleic acid with a new or enhanced feature (e.g., improved stability).

Modified Backbones and Modified Inter-Nucleoside Linkages

Examples of suitable nucleic acids containing modifications include nucleic acids containing modified backbones, bases, sugars, or non-natural internucleoside linkages. Nucleic acids (having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Modified Sugar Moieties

A subject nucleic acid can also include one or more substituted sugar moieties. Suitable polynucleotides comprise a sugar substituent group selected from: OH; H; F; O-, S-, or N-alkyl; O-, S-, or N-alkenyl; O-, S- or N-alkynyl; or O-alkyl-Co-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)·OCH₃, O(CH₂)ₙNH₂, O(CH₂)·CH₃, O(CH₂)ₙONH₂, and O(CH₂)ₙON((CH₂)·CH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂CH₂NH₂), allyl (—CH₂—CH═CH₂), —O-allyl CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Modifications as disclosed herein can be incorporated at various positions of the RNA scaffold such as at the tetra loop of a sgRNA, the repeat:anti-repeat region of the crRNA:tracrRNA component, at any position of the tracrRNA e.g. at the 5′end, 3′end, stem loop 1, 2 or 3, and at the RNA motif. Modifications disclosed herein include but are not limited to an extension of the repeat anti-repeat of a sgRNA or crRNA:tracrRNA of the 2-part component, positioning of the RNA motif at the 3′end of the tracrRNA motif, linker linking the RNA motif to the CRISPR motif, modifying the RNA motif's nucleotides and extending the RNA motif.

### Positioning of the RNA Motif

The RNA motif may be positioned at various positions of the RNA scaffold as described in Example 1. The RNA scaffold of the present invention may have one MS2 RNA motif or may have two MS2 RNA motif. The RNA motif e.g. the MS2 aptamer can be positioned at the 3′ end of the tracrRNA, at the tetra loop of the sgRNA, at stem loop 2 of the tracrRNA and at the stem loop 3 of the tracrRNA. The positioning of the aptamer such as the MS2 aptamer is crucial due to the steric hindrance that can result from the bulky loops. In a preferred embodiment, the MS2 aptamer is at the 3′end of the CRISPR motif. Advantageously, the positioning of the MS2 aptamer at the 3′end of the CRISPR motif is in space therefore reducing steric hindrance with other bulky loops of the RNA scaffold.

### Linker

The RNA motif may be linked to the tracrRNA motif via a linker. The linker may be a single-stranded RNA or a chemical linkage. The single stranded RNA linker may be 2, 3, 4, 5, 6, 7 or more than 7 nucleotides. Advantageously, the linker sequence provides flexibility to the RNA scaffold. The linker sequence may include GC nucleotides.

### Modifying the RNA Motif's Nucleotides

Modifications may be made to the RNA motif e.g. aptamer sequence. In a preferred embodiment, the RNA motif(s) comprises one or more modifications. For example a suitable modification is to the C-5 and F-5 aptamer mutant. In a preferred embodiment, the modification to the aptamer is a substitution of the Adenine to 2-aminopurine (2-AP) at position 10. Advantageously, the substitution induces conformational changes resulting in greater affinity compared to the wild-type MS2. Whilst not wishing to be bound by any theory, it is believed that the conformational change induced by 2-AP results in hydrogen bond formation between the exocyclic amino group of the 2-AP nucleotide at position 10 and the carbonyl the B59 at the backbone. It is thought that replacing the MS2 hairpin sequence with the higher affinity MS2 sequences will result in increased gene editing efficiencies because substituting amino acids helps to order the RNA stem loop into a conformation that is better recognised by the coat protein.

Suitable modifications to the RNA motif are listed above, such as 2′ deoxy-2-aminopurine, 2′ribose-2-aminopurine, phosphorothioate mods, 2′-Omethyl mods, 2′-Fluro mods and LNA mods. Advantageously, the modifications help to increase stability and promote stronger bonds/folding structure of the desired hairpin.

Other suitable modifications may be at the 5′ end and/or the 3′ end of the one or more RNA motif(s).

### Extension of RNA Motif

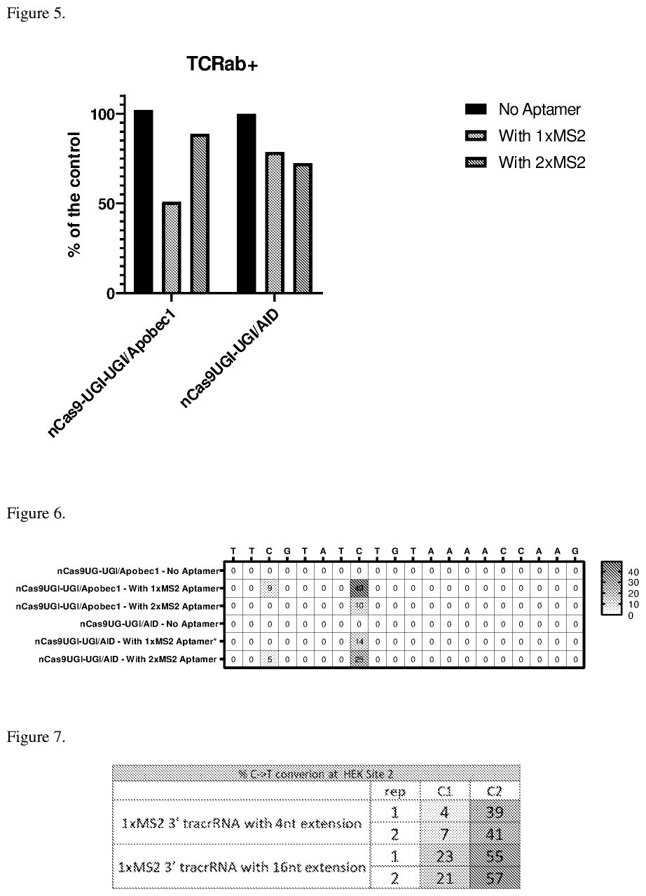

The length of the RNA motif extension can be variable. The extension to the RNA motif can range from 2-24 nucleotides. The extension to the RNA motif can be more than 24 nucleotides. FIG. 3A-D illustrates a number of extensions to the recruiting RNA motif relative to the wild-type MS2 and the sequences for the extension are show below. FIG. 3A is a 4 nucleotide (2 bp) extension which results in the stem having 23 nucleotides in total length (SEQ ID NO: 21). FIG. 3B is a 10 nucleotide (5 bp) extension which results in the stem having 29 nucleotides in total length (SEQ ID NO: 22). FIG. 3C is a 16 nucleotide (8 bp) extension which results in the stem having 35 nucleotides in total length (SEQ ID NO: 23). FIG. 3D is a 26 nucleotide (13 bp) extension which results in the stem having 45 nucleotides in total length (SEQ ID NO: 24). Advantageously, the extension of the RNA motif increases flexibility of the motif. The extension to the RNA motif may be a double-stranded or a single-stranded extension. Double-stranded extension provides greater stabilization of the RNA scaffold. In a preferred embodiment, the extension of the RNA motif is double-stranded.

### Sequences for RNA Motif Extension:

(SEQ ID NO: 21)
GCGCACAUGAGGAUCACCCAUGUGC-4 nt extension
(SEQ ID NO: 22)
GCGAGCGACAUGAGGAUCACCCAUGUCGCUC-10 nt extension
(SEQ ID NO: 23)
GCCACGAGCGACAUGAGGAUCACCCAUGUCGCUCGUG-
16 nt extension
(SEQ ID NO: 24)
GCCGUCAGACGAGCGACAUGAGGAUCACCCAUGUCGCUCGUCUGACG-
26 nt extension

### Key:

GC linker is underlined, nucleotide extension is shown in bold and the aptamer is in italics.

### The Repeat: Anti-Repeat Region

The crRNA and tracrRNA can be provided as a sgRNA or as two separate components. The crRNA hybridises to the tracrRNA via a repeat:anti-repeat region. The repeat region of the crRNA hybridises to the anti-repeat region of the tracrRNA. The repeat:anti-repeat region may be extended to increase the flexibility, proper folding and stability of the component. The repeat:anti-repeat region can be extended by 2, 3, 4, 5, 6, 7 bases or more than 7 bases at either side of the region. The repeat:anti-repeat region can be extended by 14 nucleotides in total. The repeat:anti-repeat may also comprise other modification as disclosed above.

### Combination of Modifications

The RNA scaffold may have one or more of the above mentioned modifications. The one or more modifications to the RNA scaffold is one or more of the above mentioned modifications, such as an extension to the repeat:anti-repeat region, an extension to the recruiting RNA motif, or a substitution of a nucleotide to 2AP. The one or more modification can be on the different components of the RNA scaffold e.g. extension of repeat:anti-repeat region of the sgRNA, or the 2-part crRNA:tracrRNA, and extension of the RNA motif. The one or more modifications or can be on the same component of the RNA scaffold, e.g. extension of the RNA motif and substitution of the RNA motif's nucleotides. The modifications may be two or more, three or more, four or more, or five or more. In one embodiment, the modification may be the extension of the RNA motif and/or may be the substitution of the RNA motif's nucleotide. For example, the modification may be the extension of the RNA motif or the substitution of the RNA motif's nucleotide. In other cases, the RNA motif may have the length extended and the nucleotide substitution.

Aptamer

In some embodiments, the aptamer binding protein can be a wild-type protein, a mutant of a wild-type protein or variants thereof. An example of a RNA motif as used herein is the MS2 aptamer. The RNA motif(s) bind to an aptamer binding molecule. The MS2 motif specifically binds to the MS2 bacteriophage coat protein (MCP). In vitro selection process was repeated yielding a series of aptamer families. Two of the aptamer family members include MS2 C-5 mutant and MS2 F-5 mutant. One of the significant differences between the wild-type MS2 and the C-5 and F-5 mutants is the substitution of the Uracil nucleotide to Cytosine at position 5 of the aptamer loop. The F-5 mutant has been reported to have higher affinity for the coat protein compared to the wild-type and other members of the aptamer family. Suitably, both C-5 mutants and F-5 mutants are used as aptamers in the present invention. In one embodiment, the MS2 aptamer is a wild-type MS2, a mutant MS2 or variants thereof. In another embodiment, the MS2 aptamer comprises a C-5 and/or F-5 mutation. The MS2 protein linked to the CRISPR motif can be a single-copy (i.e. one MS2 loop) or a double-copy (i.e. two MS2 loops). In a preferred embodiment, the RNA scaffold has one RNA motif. In other embodiments, the RNA scaffold has more than one, more than two, more than three RNA motifs. In other embodiments, the RNA scaffold has two RNA motifs.

c. Effector Module

The third component of the platform disclosed in this invention is a non-nuclease effector. The effector module as disclosed herein comprises an RNA binding domain capable of binding to the RNA motif and an effector domain. The effector domain as used herein include but are not limited to enzymes, reporters, tags, molecules, proteins, particulates, nano particles. In one embodiment, the effector domain is a DNA modification enzyme.

The effector is not a nuclease and does not have any nuclease activity but can have the activity of other types of DNA modifying enzymes, for example base editing. Examples of the enzymatic activity include, but are not limited to, deamination activity, methyltransferase activity, demethylase activity, DNA repair activity, DNA damage activity, dismutase activity, nickase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, the effector has the activity of cytidine deaminases (e.g., AID, APOBEC3G), adenosine deaminases (e.g., ADA), DNA methyltransferases, and DNA demethylases. In some embodiments, the effectors are from different vertebrate animal species have distinct activity properties.

In preferred embodiments, this third component is a conjugate or a fusion protein that has an RNA-binding domain and an effector domain. These two domains can be joined via a linker.

In some embodiments, no effector is needed in some cell types (e.g., cancer lines over-expressing deaminases). In that case, endogenous effector (e.g. APOBEC, AID, etc) can be gene-edited to include the recruitment module, so no exogenous editor is needed. This is applicable to cell types that express the editor of interest—e.g., lymphoid (B+T cells) and certain cancer cells. In addition, the nickase activity does not have to come from the Cas module but can be recruited from the effectors—for example, dCas9 can have an aptamer to recruit both the nickase and editor via the same gRNA recruitment. The effector protein as used herein may be a wild-type, genetically engineered or a chimeric enzyme.

### RNA-Binding Domain

Although various RNA-binding domains can be used in this invention, the RNA-binding domain of Cas protein (such as Cas9) or its variant (such as dCas9) should not be used. As mentioned above, the direct fusion to dCas9, which anchors to DNA in a defined conformation, would hinder the formation of a functional oligomeric enzyme complex at the right location. Instead, the present invention takes advantages of various other RNA motif-RNA binding protein binding pairs. Examples include those listed in Table 2.

In this way, the effector protein can be recruited to the target site through RNA-binding domain's ability to bind to the recruiting RNA motif. Due to the flexibility of RNA scaffold mediated recruitment, a functional monomer, as well as dimer, tetramer, or oligomer could be formed relatively easily near the target DNA or RNA sequence.

Effector Domain

The effector component comprises an activity portion, i.e., an effector domain. In one embodiment, the effector domain as used herein include but are not limited to enzymes, reporters, tags, molecules, proteins, particulates, nano particles In some embodiments, the effector domain comprises the naturally occurring activity portion of a non-nuclease protein (e.g., deaminases). In other embodiments, the effector domain comprises a modified amino acid sequence (e.g., substitution, deletion, insertion) of a naturally occurring activity portion of a non-nuclease protein. The effector domain has an enzymatic activity. Examples of this activity include deamination activity, methyltransferase activity, demethylase activity, DNA repair activity, DNA damage activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, DNA methylation, histone acetylation activity, or histone methylation activity. Some modifications in non-nuclease protein (e.g., deaminases) can help reduce off-target effect. For example, as described below, one can reduce the recruitment of AID to off-target sites by mutating Ser38 in AID to Ala.

Linker

The above-mentioned two domains as well as others as disclosed herein can be joined by means of linkers, such as, but not limited to chemical modification, peptide linkers, chemical linkers, covalent or non-covalent bonds, or protein fusion or by any means known to one skilled in the art. The joining can be permanent or reversible. See for example U.S. Pat. Nos. 4,625,014, 5,057,301 and 5,514,363, US Application Nos. 20150182596 and 20100063258, and WO2012142515, the contents of which are incorporated herein in their entirety by reference. In some embodiments, several linkers can be included in order to take advantage of desired properties of each linker and each protein domain in the conjugate. For example, flexible linkers and linkers that increase the solubility of the conjugates are contemplated for use alone or with other linkers. Peptide linkers can be linked by expressing DNA encoding the linker to one or more protein domains in the conjugate. Linkers can be acid cleavable, photocleavable and heat sensitive linkers. Methods for conjugation are well known by persons skilled in the art and are encompassed for use in the present invention.

In some embodiments, the RNA-binding domain and the effector domain can be joined by a peptide linker. Peptide linkers can be linked by expressing nucleic acid encoding in frame the two domains and the linker. Optionally the linker peptide can be joined at either or both of the amino terminus and carboxy terminus of the domains. In some examples, a linker is an immunoglobulin hinge region linker as disclosed in U.S. Pat. Nos. 6,165,476, 5,856,456, US Application Nos. 20150182596 and 2010/0063258 and International Application WO2012/142515, each of which are incorporated herein in their entirety by reference.

Other Domains

The effector fusion protein can comprise other domains. In certain embodiments, the effector fusion protein can comprise at least one nuclear localization signal (NLS). In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

In some embodiments, the fusion protein can comprise at least one cell-penetrating domain to facilitate delivery of the protein into a target cell. In one embodiment, the cell-penetrating domain can be a cell-penetrating peptide sequence. Various cell-penetrating peptide sequences are known in the art and examples include that of the HIV-1 TAT protein, TLM of the human HBV, Pep-1, VP22, and a polyarginine peptide sequence.

In still other embodiments, the fusion protein can comprise at least one marker domain. Non-limiting examples of marker domains include fluorescent proteins, purification tags, and epitope tags. In some embodiments, the marker domain can be a fluorescent protein. In other embodiments, the marker domain can be a purification tag and/or an epitope tag. See, e.g., US 20140273233.

In one embodiment, AID was used as an example to illustrate how the system works. AID is a cytidine deaminase that can catalyze the reaction of deamination of cytidine in the context of DNA or RNA. When brought to the targeted site, AID changes a C base to U base. In dividing cells, this could lead to a C to T point mutation. Alternatively, the change of C to U could trigger cellular DNA repair pathways, mainly excision repair pathway, which will remove the mismatching U-G base-pair, and replace with a T-A, A-T, C-G, or G-C pair. As a result, a point mutation would be generated at the target C-G site. As excision repair pathway is present in most, if not all, somatic cells, recruitment of AID to the target site can correct a C-G base pair to others. In that case, if a C-G base pair is an underlying disease-causing genetic mutation in somatic tissues/cells, the above-described approach can be used to correct the mutation and thereby treat the disease.

By the same token, if an underlying disease causing genetic mutation is an A-T base pair at a specific site, one can use the same approach to recruit an adenosine deaminase to the specific site, where adenosine deaminase can correct the A-T base pair to others. Other effector enzymes are expected to generate other types of changes in base-pairing. A non-exhaustive list of examples of DNA/RNA modifying enzymes is detailed in Table 3.

TABLE 3
Examples of effector proteins that can be used in this invention

| | Genetic | Effector protein |
|---|---|---|
| Enzyme type | change | abbreviated |
| Cytidine | C→U/T | A\|I |
| deaminase | | APOBEC1 |
| | | APOBEC3A |
| | | APOBEC3B |
| | | APOBEC3C |
| | | APOBEC3D |
| | | APOBEC3F |
| | | APOBEC3G |
| | | APOBEC3H |
| | | CDA |
| Adenosine | A→I/G | ADA |
| deaminase | | ADAR1 |
| | | ADAR2 |
| | | ADAR3 |
| | | tadA |
| DNA Methyl | C→Met-C | Dnmt1 |
| transferase | | Dnmt3a |
| | | Dnmt3b |
| Demethylase | Met-C→ C | Tet1 |
| | | Tet2 |
| | | TDG |

Effector protein full names:
AID: activation induced cytidine deaminase, a.k.a AICDA
APOBEC1: apolipoprotein B mRNA editing enzyme, catalytic polypeptide-like 1.
APOBEC3A: apolipoprotein B mRNA editing enzyme, catalytic polypeptide-like 3A
APOBEC3B: apolipoprotein B mRNA editing enzyme, catalytic polypeptide-like 3B
APOBEC3C: apolipoprotein B mRNA editing enzyme, catalytic polypeptide-like 3C
APOBEC3D: apolipoprotein B mRNA editing enzyme, catalytic polypeptide-like 3D
APOBEC3F: apolipoprotein B mRNA editing enzyme, catalytic polypeptide-like 3F
APOBEC3G: apolipoprotein B mRNA editing enzyme, catalytic polypeptide-like 3G
APOBEC3H: apolipoprotein B mRNA editing enzyme, catalytic polypeptide-like 3H
CDA: Cytidine deaminase
ADA: adenosine deaminase
ADAR1: adenosine deaminase acting on RNA 1
ADAR2: adenosine deaminase acting on RNA 2
ADAR3: adenosine deaminase acting on RNA 3
tadA: tRNA-specific adenosine deaminase
Dnmt1: DNA (cytosine-5-)-methyltransferase 1
Dnmt3a: DNA (cytosine-5-)-methyltransferase 3 alpha
Dnmt3b: DNA (cytosine-5-)-methyltransferase 3 beta
Tet1: ten-eleven translocation 1
Tet2: ten-eleven translocation 2
Tdg: thymine DNA glycosylase

The above-described three specific components constitute the technological platform. Each component could be chosen from the list in Table 1-3 respectively to achieve a specific therapeutic/utility goal.

An RNA scaffold mediated recruitment system could be constructed using (i) dCas9/nCas9 from S. pyogenes as the sequence targeting protein, (ii) an RNA scaffold containing a crRNA comprising a guide RNA sequence, a tracrRNA, and a RNA motif e.g. MS2 operator motif, and (iii) an effector module containing a human AID fusing to MS2 operator binding protein MCP. The sequences for the components are listed below:

S. pyogenes dCas9 protein sequence
(SEQ ID NO: 25)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKWDELVKVMGRHKPENIVIEMARENQ
TTQKGQKNSRERMKRIEEGIKELGSQILKEHPVENTQLQNEKLYLYYLQNGRDMYVDQELDINR
LSDYDVDAIVPQSFLKDDSIDNKVLTRSDKNRGKSDNVPSEEWKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD
(Residues underlined: D10A (D → A), H840A (H → A) active site mutants)
Cas9 D10A Protein (residues underlined: D10A,)
(SEQ ID NO: 26)
DKKYSIGLAIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYN
QLFEENPINASGVDAKAILSARLSKSRRLENLIAQLPGEKKNGLFGNLIALSLGLTPNFKSNFD
LAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQT
TQKGQKNSRERMKRIEEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKRMLASA
GELQKGNELALPSKYVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTIDRKRYTSTKEVLDA
TLIHQSITGLYETRIDLSQLGGD
DNA encoding Cas9D1 OA Protein (29A > C)
(SEQ ID NO: 27)
ATGGATAAAAAGTATTCTATTGGTTTAGCCATCGGCACTAATTCCGTTGGATGGGCTGTCATAA
CCGATGAATACAAAGTACCTTCAAAGAAATTTAAGGTGTTGGGGAACACAGACCGTCATTCGAT
TAAAAAGAATCTTATCGGTGCCCTCCTATTCGATAGTGGCGAAACGGCAGAGGCGACTCGCCTG
AAACGAACCGCTCGGAGAAGGTATACACGTCGCAAGAACCGAATATGTTACTTACAAGAAATTT
TTAGCAATGAGATGGCCAAAGTTGACGATTCTTTCTTTCACCGTTTGGAAGAGTCCTTCCTTGT
CGAAGAGGACAAGAAACATGAACGGCACCCCATCTTTGGAAACATAGTAGATGAGGTGGCATAT
CATGAAAAGTACCCAACGATTTATCACCTCAGAAAAAAGCTAGTTGACTCAACTGATAAAGCGG
ACCTGAGGTTAATCTACTTGGCTCTTGCCCATATGATAAAGTTCCGTGGGCACTTTCTCATTGA
GGGTGATCTAAATCCGGACAACTCGGATGTCGACAAACTGTTCATCCAGTTAGTACAAACCTAT
AATCAGTTGTTTGAAGAGAACCCTATAAATGCAAGTGGCGTGGATGCGAAGGCTATTCTTAGCG
CCCGCCTCTCTAAATCCCGAGGGCTAGAAAACCTGATCGCACAATTACCCGGAGAGAAGAAAAA
TGGGTTGTTCGGTAACCTTATAGCGCTCTCACTAGGCCTGACACCAAATTTTAAGTCGAACTTC
GACTTAGCTGAAGATGCCAAATTGCAGCTTAGTAAGGACACGTACGATGACGATCTCGACAATC
TACTGGCACAAATTGGAGATCAGTATGCGGACTTATTTTTGGCTGCCAAAAACCTTAGCGATGC
AATCCTCCTATCTGACATACTGAGAGTTAATACTGAGATTACCAAGGCGCCGTTATCCGCTTCA
ATGATCAAAAGGTACGATGAACATCACCAAGACTTGACACTTCTCAAGGCCCTAGTCCGTCAGC
AACTGCCTGAGAAATATAAGGAAATATTCTTTGATCAGTCGAAAAACGGGTACGCAGGTTATAT
TGACGGCGGAGCGAGTCAAGAGGAATTCTACAAGTTTATCAAACCCATATTAGAGAAGATGGAT
GGGACGGAAGAGTTGCTTGTAAAACTCAATCGCGAAGATCTACTGCGAAAGCAGCGGACTTTCG
ACAACGGTAGCATTCCACATCAAATCCACTTAGGCGAATTGCATGCTATACTTAGAAGGCAGGA
GGATTTTTATCCGTTCCTCAAAGACAATCGTGAAAAGATTGAGAAAATCCTAACCTTTCGCATA
CCTTACTATGTGGGACCCCTGGCCCGAGGGAACTCTCGGTTCGCATGGATGACAAGAAAGTCCG
AAGAAACGATTACTCCATGGAATTTTGAGGAAGTTGTCGATAAAGGTGCGTCAGCTCAATCGTT
CATCGAGAGGATGACCAACTTTGACAAGAATTTACCGAACGAAAAAGTATTGCCTAAGCACAGT
TTACTTTACGAGTATTTCACAGTGTACAATGAACTCACGAAAGTTAAGTATGTCACTGAGGGCA
TGCGTAAACCCGCCTTTCTAAGCGGAGAACAGAAGAAAGCAATAGTAGATCTGTTATTCAAGAC
CAACCGCAAAGTGACAGTTAAGCAATTGAAAGAGGAGTAGTTTAAGAAAATTGAATGCTTCGAT
TCTGTCGAGATCTCCGGGGTAGAAGATCGATTTAATGCGTCACTTGGTACGTATCATGACCTCC
TAAAGATAATTAAAGATAAGGACTTCCTGGATAACGAAGAGAATGAAGATATCTTAGAAGATAT
AGTGTTGACTCTTACCCTCTTTGAAGATCGGGAAATGATTGAGGAAAGACTAAAAACATACGCT
CACCTGTTCGACGATAAGGTTATGAAACAGTTAAAGAGGCGTCGCTATACGGGCTGGGGACGAT
TGTCGCGGAAACTTATCAACGGGATAAGAGACAAGCAAAGTGGTAAAACTATTCTCGATTTTCT
AAAGAGCGACGGCTTCGCCAATAGGAACTTTATGCAGCTGATCCATGATGACTCTTTAACCTTC
AAAGAGGATATACAAAAGGCACAGGTTTCCGGACAAGGGGACTCATTGCACGAACATATTGCGA
ATCTTGCTGGTTCGCCAGCCATCAAAAAGGGCATACTCCAGACAGTCAAAGTAGTGGATGAGCT
AGTTAAGGTCATGGGACGTCACAAACCGGAAAACATTGTAATCGAGATGGCACGCGAAAATCAA
ACGACTCAGAAGGGGCAAAAAAACAGTCGAGAGCGGATGAAGAGAATAGAAGAGGGTATTAAAG
AACTGGGCAGCCAGATCTTAAAGGAGCATCCTGTGGAAAATACCCAATTGCAGAACGAGAAACT
TTACCTCTATTACCTACAAAATGGAAGGGACATGTATGTTGATCAGGAACTGGACATAAACCGT
TTATCTGATTACGACGTCGATCACATTGTACCCCAATCCTTTTTGAAGGACGATTCAATCGACA
ATAAAGTGCTTACACGCTCGGATAAGAACCGAGGGAAAAGTGACAATGTTCCAAGCGAGGAAGT
CGTAAAGAAAATGAAGAACTATTGGCGGCAGCTCCTAAATGCGAAACTGATAACGCAAAGAAAG
TTCGATAACTTAACTAAAGCTGAGAGGGGTGGCTTGTCTGAACTTGACAAGGCCGGATTTATTA
AACGTCAGCTCGTGGAAACCCGCCAAATCACAAAGCATGTTGCACAGATACTAGATTCCCGAAT
GAATACGAAATACGACGAGAACGATAAGCTGATTCGGGAAGTCAAAGTAATCACTTTAAAGTCA
AAATTGGTGTCGGACTTCAGAAAGGATTTTCAATTCTATAAAGTTAGGGAGATAAATAACTACC
ACCATGCGCACGACGCTTATCTTAATGCCGTCGTAGGGACCGCACTCATTAAGAAATACCCGAA
GCTAGAAAGTGAGTTTGTGTATGGTGATTACAAAGTTTATGACGTCCGTAAGATGATCGCGAAA
AGCGAACAGGAGATAGGCAAGGCTACAGCCAAATACTTCTTTTATTCTAACATTATGAATTTCT
TTAAGACGGAAATCACTCTGGCAAACGGAGAGATACGCAAACGACCTTTAATTGAAACCAATGG
GGAGACAGGTGAAATCGTATGGGATAAGGGCCGGGACTTCGCGACGGTGAGAAAAGTTTTGTCC
ATGCCCCAAGTCAACATAGTAAAGAAAACTGAGGTGCAGACCGGAGGGTTTTCAAAGGAATCGA
TTCTTCCAAAAAGGAATAGTGATAAGCTCATCGCTCGTAAAAAGGACTGGGACCCGAAAAAGTA
CGGTGGCTTCGATAGCCCTACAGTTGCCTATTCTGTCCTAGTAGTGGCAAAAGTTGAGAAGGGA
AAATCCAAGAAACTGAAGTCAGTCAAAGAATTATTGGGGATAACGATTATGGAGCGCTCGTCTT
TTGAAAAGAACCCCATCGACTTCCTTGAGGCGAAAGGTTACAAGGAAGTAAAAAAGGATCTCAT
AATTAAACTACCAAAGTATAGTCTGTTTGAGTTAGAAAATGGCCGAAAACGGATGTTGGCTAGC
GCCGGAGAGCTTCAAAAGGGGAACGAACTCGCACTACCGTCTAAATACGTGAATTTCCTGTATT
TAGCGTCCCATTACGAGAAGTTGAAAGGTTCACCTGAAGATAACGAACAGAAGCAACTTTTTGT
TGAGCAGCACAAACATTATCTCGACGAAATCATAGAGCAAATTTCGGAATTCAGTAAGAGAGTC
ATCCTAGCTGATGCCAATCTGGACAAAGTATTAAGCGCATACAACAAGCACAGGGATAAACCCA
TACGTGAGCAGGCGGAAAATATTATCCATTTGTTTACTCTTACCAACCTCGGCGCTCCAGCCGC
ATTCAAGTATTTTGACACAACGATAGATCGCAAACGATACACTTCTACCAAGGAGGTGCTAGAC
GCGACACTGATTCACCAATCCATCACGGGATTATATGAAACTCGGATAGATTTGTCACAGCTTG
GGGGTGAC

RNA scaffold expression cassette (S. pyogenes), containing a 20-nucleotide programmable sequence, a CRISPR RNA motif (tracrRNA), and an MS2 operator motif:

SEQ ID NO: 28
N₂₀GTTTTAGAGCTAGAAATAGCAAGTTAAAATAAGGCTAGTCCGTTATC
AACTTGAAAAAGTGGCACCGAGTCGGTGC [symbol]
TTTTTTT

(N₂₀: programmable sequence; Underlined: CRISPR RNA motif (tracrRNA); Bold: MS2 motif; Italic: terminator; Bold and italics: GC linker; Bold and underlined: extension to the MS2)

The above RNA scaffold containing one MS2 loop (1×MS2). Shown below is an RNA scaffold containing two MS2 loops (2×MS2), where MS2 scaffolds are underlined:

SEQ ID NO: 29
GTTTTAGAGCTAGAAATAGCAAGTTAAAATAAGGCTAGTCCGTTATCAA
CTTGAAAAAGTGGCACCGAGTCGGTGCgggagcACATGAGGATCACCCA
TGTgccacgagcgACATGAGGATCACCCATGTcgctcgtgttccc
TTTTTTT

Effector AID-MCP Fusion:

SEQ ID NO: 30
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT

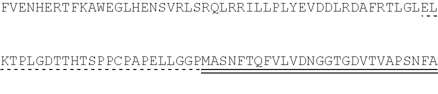

NGIAEWISSNSRSQAYKVTCSVRQSSAQNRKYTIKVEVPKGAWRSYLNME
LTIPIFATNSDCELIVKAMQGLLKDGNPIPSAIAANSGIY
Key:

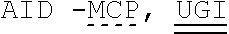

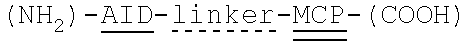

Like the Cas protein described above, the non-nuclease effector can also be obtained as a recombinant polypeptide. Techniques for making recombinant polypeptides are known in the art.

As described herein, by mutating Ser38 to Ala in AID one can reduce the recruitment of AID to off-target sites. Listed below are the DNA and protein sequences of both wild type AID as well as AID_S38A (phosphorylation null, pnAID):

wtAID cDNA (Ser38 codon in bold and underlined,):
SEQ ID NO: 31
ATGGACAGCCTCTTGATGAACCGGAGGAAGTTTCTTTACCAATTCAAAAATGTCCGCTGGGCTA
AGGGTCGGCGTGAGACCTACCTGTGCTACGTAGTGAAGAGGCGTGACAGTGCTACATCCTTTTC
ACTGGACTTTGGTTATCTTCGCAATAAGAACGGCTGCCACGTGGAATTGCTCTTCCTCCGCTAC
ATCTCGGACTGGGACCTAGACCCTGGCCGCTGCTACCGCGTCACCTGGTTCACCTCCTGGAGCC
CCTGCTACGACTGTGCCCGACATGTGGCCGACTTTCTGCGAGGGAACCCCAACCTCAGTCTGAG
GATCTTCACCGCGCGCCTCTACTTCTGTGAGGACCGCAAGGCTGAGCCCGAGGGGCTGCGGCGG
CTGCACCGCGCCGGGGTGCAAATAGCCATCATGACCTTCAAAGATTATTTTTACTGCTGGAATA
CTTTTGTAGAAAACCATGAAAGAACTTTCAAAGCCTGGGAAGGGCTGCATGAAAATTCAGTTCG
TCTCTCCAGACAGCTTCGGCGCATCCTTTTGCCCCTGTATGAGGTTGATGACTTACGAGACGCA
TTTCGTACTTTGGGACTT
wtAID protein (Ser38 in bold and underlined,):
SEQ ID NO: 32
MDSLLMNRRKFLYQFKNVRWAKGRRETYLCYVVKRRDSATSFSLDFGYLRNKNGCHVELLFLRY
ISDWDLDPGRCYRVTWFTSWSPCYDCARHVADFLRGNPNLSLRIFTARLYFCEDRKAEPEGLRR
LHRAGVQIAIMTFKDYFYCWNTFVENHERTFKAWEGLHENSVRLSRQLRRILLPLYEVDDLRDA
FRTLGL
AID_S38A cDNA (S38A mutation in bold and underlined,)
SEQ ID NO: 33
ATGGACAGCCTCTTGATGAACCGGAGGAAGTTTCTTTACCAATTCAAAAATGTCCGCTGGGCTA
AGGGTCGGCGTGAGACCTACCTGTGCTACGTAGTGAAGAGGCGTGACGCCGCTACATCCTTTTC
ACTGGACTTTGGTTATCTTCGCAATAAGAACGGCTGCCACGTGGAATTGCTCTTCCTCCGCTAC
ATCTCGGACTGGGACCTAGACCCTGGCCGCTGCTACCGCGTCACCTGGTTCACCTCCTGGAGCC
CCTGCTACGACTGTGCCCGACATGTGGCCGACTTTCTGCGAGGGAACCCCAACCTCAGTCTGAG
GATCTTCACCGCGCGCCTCTACTTCTGTGAGGACCGCAAGGCTGAGCCCGAGGGGCTGCGGCGG
CTGCACCGCGCCGGGGTGCAAATAGCCATCATGACCTTCAAAGATTATTTTTACTGCTGGAATA
CTTTTGTAGAAAACCATGAAAGAACTTTCAAAGCCTGGGAAGGGCTGCATGAAAATTCAGTTCG
TCTCTCCAGACAGCTTCGGCGCATCCTTTTGCCCCTGTATGAGGTTGATGACTTACGAGACGCA
TTTCGTACTTTGGGACTT
AID_S38A protein (S38A mutation in bold and underlined,)

SEQ ID NO: 34
MDSLLMNRRKFLYQFKNVRWAKGRRETYLCYVVKRRDAATSFSLDFGYLRNKNGCHVELLFLRY
LSDWDLDPGRCYRVTWFTSWSPCYDCARHVADFLRGNPNLSLRIFTARLYFCEDRKAEPEGLRR
LHRAGVQIAIMTFKDYFYCWNTFVENHERTFKAWEGLHENSVRLSRQLRRILLPLYEVDDLRDA
FRTLGL

Exemplary Sequences

Shown below are a number of exemplary sequences developed in this study.

Protein sequence of ^{A}RNA scaffold mediated recruitment system nu construct
(SEQ ID NO: 35):

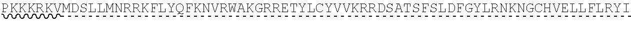

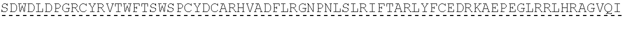

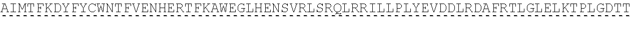

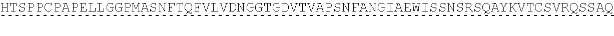

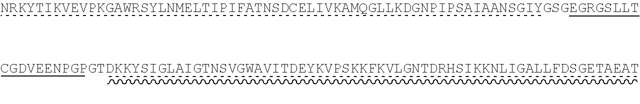

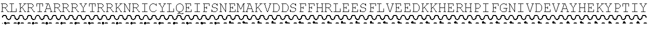

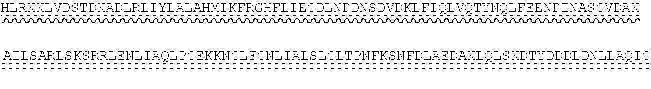

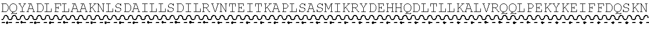

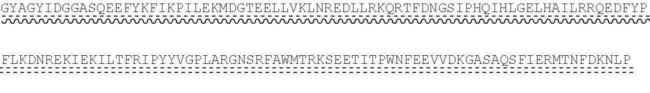

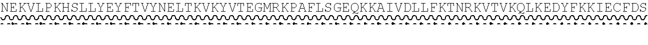

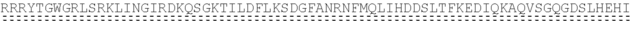

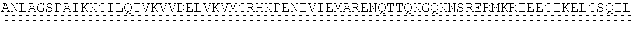

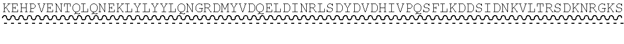

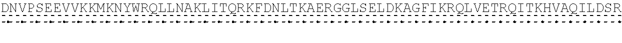

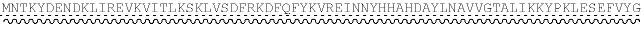

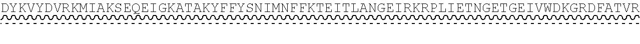

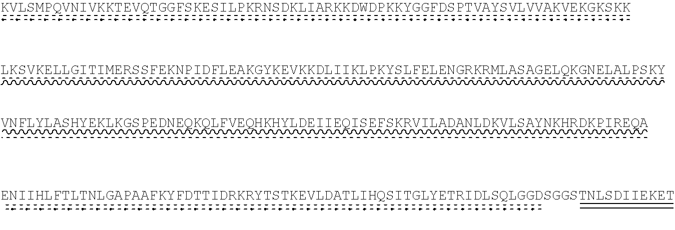

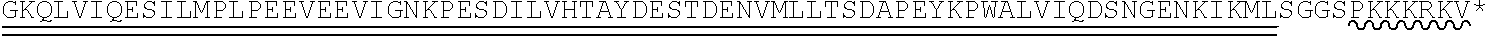

Key:

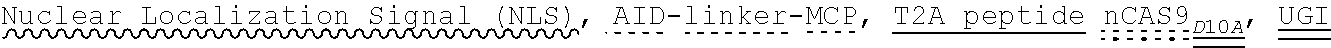

Protein sequence of ^{A}RNA scaffold mediated recruitment system nu.2 construct
(SEQ ID NO: 36):

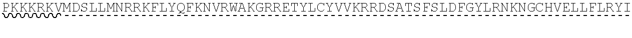

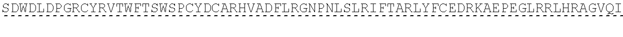

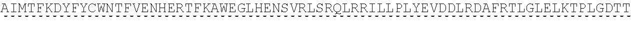

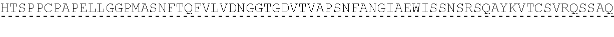

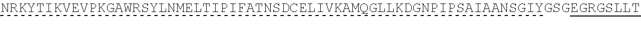

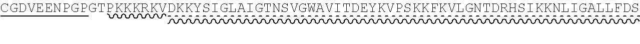

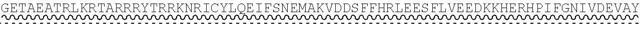

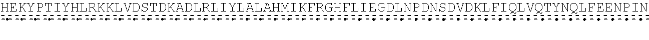

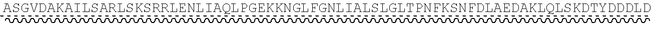

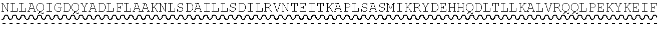

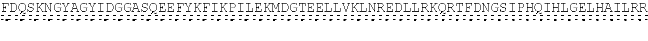

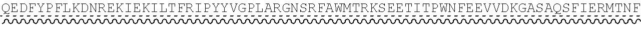

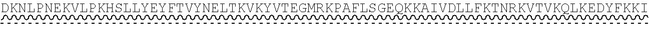

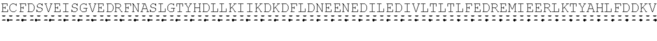

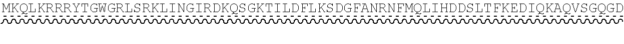

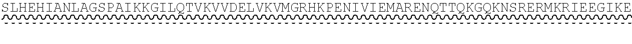

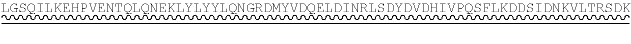

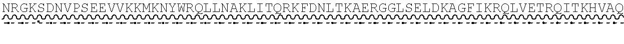

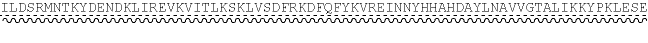

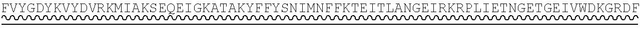

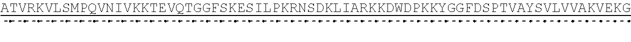

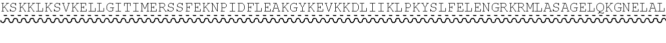

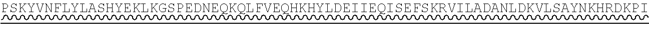

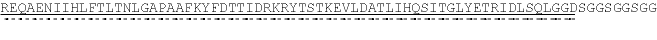

STNLSDIIEKETGKQLVIQESILMLPEEVEEVIGNKPESDILVHTAYDESTDENVMLLTSDAPEYKPWA
LVIQDSNGENKIKMLSGGSGGSGGSTNLSDIIEKETGKQLVIQESILMLPEEVEEVIGNK

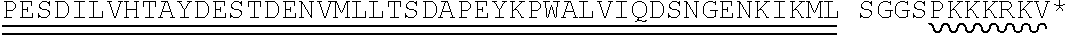

Key:

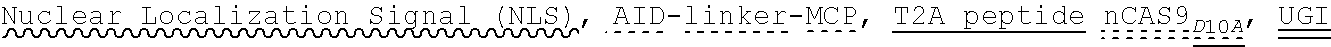

Protein sequence of RNA scaffold mediated recruitment system
(SEQ ID NO: 37):

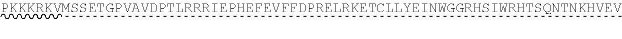

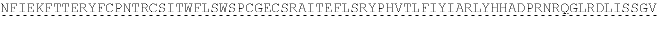

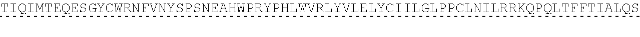

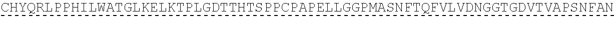

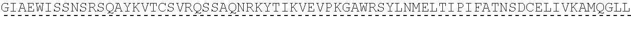

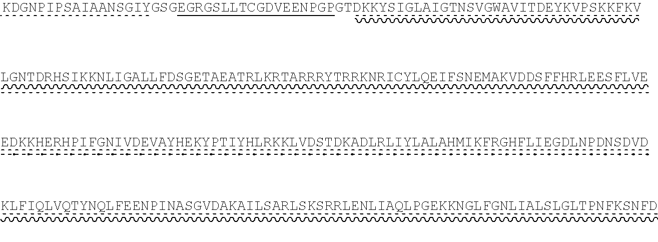

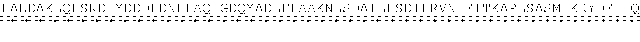

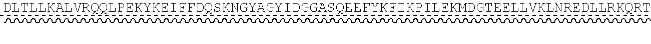

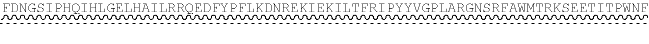

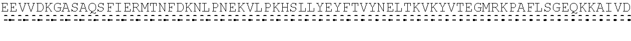

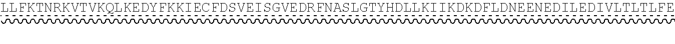

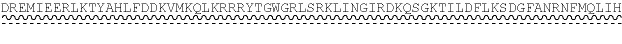

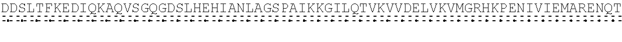

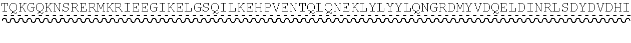

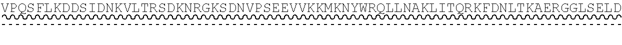

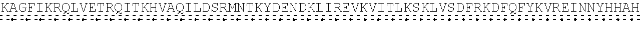

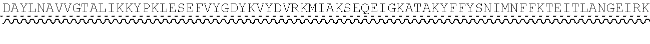

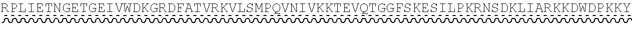

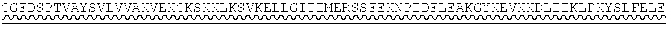

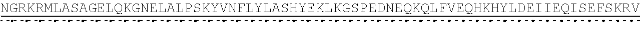

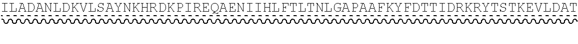

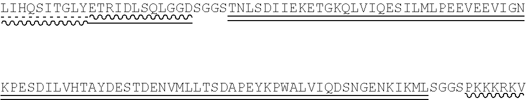

Key:

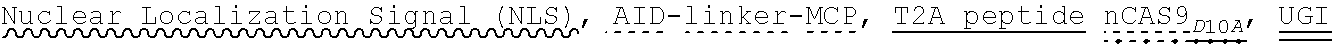

The 2×UGI base editor sequence is represented by SEQ ID NO: 186.

Shown below are a number of exemplary RNA sequence of gRNA constructs used in this study. Each contains, from the 5′ end to the 3′ end, a customizable target, a gRNA scaffold, and one or two copies of a MS2 aptamer.

1. Sequence of gRNA_MS2 construct
(SEQ ID NO: 38):
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGCgcg

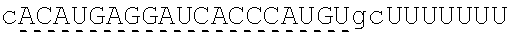

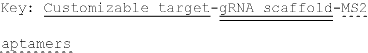

2. Sequence of gRNA_2xMS2 construct
(SEQ ID NO: 39):
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGCggga

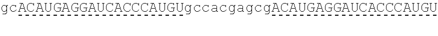

CGCTCGTGTTCCCUUUUUUU

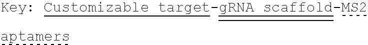

The above three components of the platform/system disclosed herein can be expressed using one, two or three expression vectors. The system can be programmed to target virtually any DNA or RNA sequence. Similar RNA scaffold recruitment system could be generated by varying the modular components of the system, including any suitable Cas orthologs, deaminase orthologs, and other DNA modification enzymes.

Cell Types/Therapeutic Uses

The RNA scaffold recruitment system of the present invention can be used to genetically modify cells including but not limited to animal cells, fungal cells and plant cells. In a preferred embodiment, the RNA scaffold of the present invention can be used to genetically modify human cells. The present invention can be applied to primary cell lines, immortalised cell lines, primary cells isolated from humans. Examples of human cells include, but are not limited to, differentiated cells or differentiating cells or stem cells. Suitable human cells include those derived from any of the three embryonic germ layers, i.e., endoderm, mesoderm, and ectoderm. For example, human cells are cells found in the following organs: skeletal muscle, skeleton, dermis of skin, connective tissue, urogenital system, heart, blood (lymph cells), and spleen (mesoderm); stomach, colon, liver, pancreas, urinary bladder; lining of urethra, epithelial parts of trachea, lungs, pharynx, thyroid, parathyroid, intestine (endoderm); or central nervous system, retina and lens, cranial and sensory, ganglia and nerves, pigment cells, head connective tissue, epidermis, hair, mammary glands (ectoderm). In a preferred embodiment, the RNA scaffold is used to genetically modify primary immune cells or immune cell lines. Immune cells include T cells, NK cells, B cells, CD34+ hematopoietic stem progenitor cells (HSPCs) and other cells involved in the production of lymphocytes and cells of blood, bone marrow, spleen, lymph nodes, and thymus. Immune cells, particularly primary immune cells either naturally occurring within a host animal or patient or derived from an induced pluripotent stem cell [iPSC] may be genetically modified. Immune cells include T cells, NK cells, B cells, pluripotent cells such as haematopoietic stem cells (HSCs) which are pluripotent cells that can differentiate into immune cells and other cells involved in the production of lymphocytes and cells of blood, bone marrow, spleen, lymph nodes, and thymus.

Provided herein are also methods for genome engineering (e.g., methods for altering or manipulating the expression of one or more genes or one or more gene products) in cells in vitro, in vivo, or ex vivo. In particular, the methods provided herein are useful for targeted base editing disruption in mammalian cells.

In another aspect, provided herein are methods for targeting diseases for base editing correction. The target sequence can be any disease-associated polynucleotide or gene, as have been established in the art. Examples of useful applications of mutation or ‘correction’ of an endogenous gene sequence include alterations of disease-associated gene mutations, alterations in sequences encoding splice sites, alterations in regulatory sequences, alterations in sequences to cause a gain-of-function mutation, and/or alterations in sequences to cause a loss-of-function mutation, and targeted alterations of sequences encoding structural characteristics of a protein.

In some cases, it will be advantageous to genetically modify a cell using the methods described herein such that cell expresses a chimeric antigen receptor (CAR) and/or T cell receptor (TCR). The “chimeric antigen receptor (CAR)” is sometimes called a “chimeric receptor”, a “T-body”, or a “chimeric immune receptor (CIR).” As used herein, the term “chimeric antigen receptor (CAR)” refers to an artificially constructed hybrid protein or polypeptide comprising an extracellular antigen binding domains of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, T cells can be engineered to express CAR specific for CD19 on B-cell lymphoma. For allogenic antitumor cell therapeutics not limited by donor-matching, cells can be engineered to knock-in nucleic acids encoding a CAR but also knocking out genes responsible for donor matching (TCR and HLA markers).

As used herein, the terms “genetically modified” and “genetically engineered” are used interchangeably and refer to a prokaryotic or eukaryotic cell that includes an exogenous polynucleotide, regardless of the method used for insertion. In some cases, the effector cell has been modified to comprise a non-naturally occurring nucleic acid molecule that has been created or modified by the hand of man (e.g., using recombinant DNA technology) or is derived from such a molecule (e.g., by transcription, translation, etc.). An effector cell that contains an exogenous, recombinant, synthetic, and/or otherwise modified polynucleotide is considered to be an engineered cell.

Cell Therapies and Ex Vivo Therapies

Various embodiments of the present invention also provide cells that are produced or used in accordance with any of the other embodiments of the present invention for use in therapy. In one embodiment, the present invention is directed to methods for generating therapeutic cells such as T cells engineered to express a Chimeric Antigen Receptor (CAR-T) or T Cell Receptor (TCR-T). The CAR-T/TCR-T cells may be derived from primary T cells or differentiated from stem cells. Suitable stem cells include, but are not limited to, mammalian stem cells such as human stem cells, including, but not limited to, hematopoietic, neural, embryonic, induced pluripotent stem cells (iPSC), mesenchymal, mesodermal, liver, pancreatic, muscle, and retinal stem cells. Other stems cells include, but are not limited to, mammalian stem cells such as mouse stem cells, e.g., mouse embryonic stem cells.

In various embodiments, the present invention may be used to knockout, base-changes, modify the expression of a single gene or multiple genes in various types of cells or cell lines, including but not limited to cells from eukaryotes e.g. human cells. The present invention may be used for multiplex modifications i.e. one or more base edits, which can be introduced simultaneously or sequentially. The technology may be used for many applications, including but not limited to knock out of genes to prevent graft versus host disease by making non-host cells non-immunogenic to the host or prevent host vs graft disease by making non-host cells resistant to attack by the host. These approaches are also relevant to generating allogenic (off-the-shelf) or autologous (patient specific) cell-based therapeutics. Such genes include, but are not limited to, the T Cell Receptor (TRAC), the major histocompatibility complex (MHC class I and class II) genes, including B2M, co-receptors (HLA-F, HLA-G), genes involved in the innate immune response (MICA, MICB, HCP5, STING, DDX41 and Toll-like-receptors (TLRs)), inflammation (NKBBiL, LTA, TNF, LTB, LST1, NCR3, AIF1), heat shock proteins (HSPA1L, HSPA1A, HSPA1B), complement cascade, regulatory receptors (NOTCH family members), antigen processing (TAP, HLA-DM, HLA-DO), increased potency or persistence (such as PD-1, CTLA-4 and other members of the B7 family of checkpoint proteins), genes involved in immunosuppressive immune cells (such as FOXP3 and Interleukin (IL)-10), genes involved in T cell interaction with the tumour microenvironment (including but not limited to receptors of cytokines such as TGFB, IL-4, IL-7, IL-2, IL-15, IL-12, IL-18, IFNgamma), genes involved in contributing to cytokine release syndrome (including but not limited to IL-6, IFNgamma, IL-8 (CXCL8), IL-10, GM-CSF, MIP-1α/β, MCP-1 (CCL2), CXCL9, and CXCL10 (IP-10), genes that code for the antigen targeted by a CAR/TCR (for example endogenous CS1 where the CAR is designed against CS1) or other genes found to be beneficial to CAR-T/TCR-T (such as TET2) or other cell based therapeutics including but not limited to CAR-NK. CAR-B etc. See, e.g., DeRenzo et al., Genetic Modification Strategies to Enhance CAR T Cell Persistence for Patients With Solid Tumors. Front. Immunol., 15 Feb. 2019.

The technology may also be used to knock down or modify genes that are involved in fratricide of immune cells, such as T cells and NK cells, or genes that alert the immune system of a patient or animal that a foreign cell, particle or molecule has entered a patient or animal, or genes encoding proteins that are current therapeutic targets used to compromise or boost an immune response, for example, CD52 and PD1, respectively.

One application is to engineer HLA alleles of bone marrow cells to increase haplotype match. The engineered cells can be used for bone marrow transplantation for treating leukemia. Another application is to engineer the negative regulatory element of fetal hemoglobin gene in hematopoietic stem cells for treating sickle cell anemia and beta-thalassemia. The negative regulatory element will be mutated and the expression of fetal hemoglobin gene is re-activated in hematopoietic stem cells, compensating the functional loss due to mutations in adult alpha or beta hemoglobin genes. A further application is to engineer iPS cells for generating allogenic therapeutic cells for various degenerative diseases including Parkinson's disease (neuronal cell loss), Type 1 diabetes (pancreatic beta cell loss). Other exemplary applications include engineering HIV infection resistant T-Cells by inactivating CCR5 gene and other genes encoding receptors required for HIV entering cells.

Type of Genetic Modifications

Accordingly, provided herein are methods for targeted disruption of transcription or translation of a target gene. In particular, the methods comprise targeted disruption of transcription or translation of a target gene via disruption of a start codon, introduction of a premature stop codon, and/or targeted disruption of intron/exon splice sites.

Using the methods described herein, one may knock-in and/or knock-out one or more genes of interest in primary cells with improved efficiency and a reduced rate of off-target indel formation. In preferred embodiments, the methods are used for multiplexed base editing comprising gene knock-in, gene knock-out, and missense mutation.

As described in the paragraphs and Examples that follow, the inventors' streamlined approach to genome engineering employs base editors (e.g., 3rd- and 4th-generation base editors, adenine base editor) for targeted gene disruption by knock-out and missense mutation and also targeted gene knock-in in the presence of a DNA donor template. The methods described herein are well-suited for studying hematopoietic cell biology and gene function, modeling diseases such as primary immunodeficiencies, as well as correcting disease-causing point mutations, and generating novel cell products (e.g., T cell products) for therapeutic applications.

Delivery of Components into Cells

Suitable methods for delivering the base editing components to cells are provided in the Examples herein below.

In embodiments provided herein the RNA scaffold is chemically synthesized RNA and is introduced into the cells by any suitable technique, e.g. such as electroporation. The base editing enzyme component and Class 2 Cas enzyme component may be introduced into the cells as mRNA or proteins.

In embodiments, components including a base editor and a guide molecule can be delivered to a cell, in vitro, ex vivo, or in vivo. In some cases, a viral or plasmid vector system is employed for delivery of base editing components described herein. Preferably, the vector is a viral vector, such as a lenti- or baculo- or preferably adeno-viral/adeno-associated viral (AAV) vectors, but other means of delivery are known (such as yeast systems, microvesicles, gene guns/means of attaching vectors to gold nanoparticles) and are contemplated. In certain embodiments, nucleic acids encoding gRNAs and base editor fusion proteins are packaged for delivery to a cell in one or more viral delivery vectors. Suitable viral delivery vectors include, without limitation, adeno-viral/adeno-associated viral (AAV) vectors, lentiviral vectors. In some cases, non-viral transfer methods as are known in the art can be used to introduce nucleic acids or proteins in mammalian cells. Nucleic acids and proteins can be delivered with a pharmaceutically acceptable vehicle, or for example, encapsulated in a liposome. Other means of delivery are known (such as yeast systems, microvesicles, gene guns/means of attaching vectors to gold nanoparticles) and are contemplated. In some cases, cells are electroporated for uptake of gRNA and base editor (e.g., BE3, BE4, ABE). In some cases, DNA donor template is delivered as Adeno-Associated Virus Type 6 (AAV6) vector by addition of viral supernatant to culture medium after introduction of the gRNA, base editor, and vector by electroporation.

Rates of insertion or deletion (indel) formation can be determined by an appropriate method. For example, Sanger sequencing or next generation sequencing (NGS) can be used to detect rates of indel formation. Preferably, the contacting results in less than 20% off-target indel formation upon base editing. The contacting results in at least 2:1 intended to unintended product upon base editing.

Expression System

To use the platform described above, it may be desirable to express one or more of the protein and RNA components from nucleic acids that encode them. This can be performed in a variety of ways. For example, the nucleic acids encoding the RNA scaffold or proteins can be cloned into one or more intermediate vectors for introducing into prokaryotic or eukaryotic cells for replication and/or transcription. Intermediate vectors are typically prokaryotic vectors, e.g., plasmids, or shuttle vectors, or insect vectors, for storage or manipulation of the nucleic acid encoding the RNA scaffold or protein for production of the RNA scaffold or protein. The nucleic acids can also be cloned into one or more expression vectors, for administration to a plant cell, animal cell, preferably a mammalian cell or a human cell, fungal cell, bacterial cell, or protozoan cell. Accordingly, the present invention provides nucleic acids that encode any of the RNA scaffold or proteins mentioned above. Preferably, the nucleic acids are isolated and/or purified.

The present invention also provides recombinant constructs or vectors having sequences encoding one or more of the RNA scaffold or proteins described above. Examples of the constructs include a vector, such as a plasmid or viral vector, into which a nucleic acid sequence of the invention has been inserted, in a forward or reverse orientation. In a preferred embodiment, the construct further includes regulatory sequences, including a promoter, operably linked to the sequence. Large numbers of suitable vectors and promoters are known to those of skill in the art, and are commercially available. Appropriate cloning and expression vectors for use with prokaryotic and eukaryotic hosts known in the art.

A vector refers to a nucleic acid molecule capable of transporting another nucleic acid to which it has been linked. The vector can be capable of autonomous replication or integration into a host DNA. Examples of the vector include a plasmid, cosmid, or viral vector. The vector of this invention includes a nucleic acid in a form suitable for expression of the nucleic acid in a host cell. Preferably, the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the host cell to be transformed, transfected, or transduced, the level of expression of RNAs or proteins desired, and the like.

Examples of expression vectors include chromosomal, non-chromosomal and synthetic DNA sequences, bacterial plasmids, phage DNA, baculovirus, yeast plasmids, vectors derived from combinations of plasmids and phage DNA, viral DNA such as vaccinia, adenovirus, fowl pox virus, and pseudorabies. However, any other vector may be used provided it is replicable and viable in the host. The appropriate nucleic acid sequence may be inserted into the vector by a variety of procedures. In general, a nucleic acid sequence encoding one of the RNAs or proteins described above can be inserted into an appropriate restriction endonuclease site(s) by procedures known in the art. Such procedures and related sub-cloning procedures are within the scope of those skilled in the art.

The vector may include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli.

The vectors for expressing the RNAs can include RNA Pol III promoters to drive expression of the RNAs, e.g., the HI, U6 or 7SK promoters. These human promoters allow for expression of RNAs in mammalian cells following plasmid transfection. Alternatively, a T7 promoter may be used, e.g., for in vitro transcription, and the RNA can be transcribed in vitro and purified.

The vector containing the appropriate nucleic acid sequences as described above, as well as an appropriate promoter or control sequence, can be employed to transform, transfect, or infect an appropriate host to permit the host to express the RNAs or proteins described above. Examples of suitable expression hosts include bacterial cells (e.g., E. coli, Streptomyces, Salmonella typhimurium), fungal cells (yeast), insect cells (e.g., Drosophila and Spodoptera frugiperda (Sf9)), animal cells (e.g., CHO, COS, and HEK 293), adenoviruses, and plant cells. The selection of an appropriate host is within the scope of those skilled in the art. In some embodiments, the present invention provides methods for producing the above mentioned RNAs or proteins by transforming, transfecting, or infecting a host cell with an expression vector having a nucleotide sequence that encodes one of the RNAs, or polypeptides, or proteins. The host cells are then cultured under a suitable condition, which allows for the expression of the RNAs or proteins.

Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

### Culturing the Cells

The method further comprises maintaining the cell under appropriate conditions such that the guide RNA guides the effector protein to the targeted site in the target sequence, and the effector domain modifies the target sequence.

In general, the cell can be maintained under conditions appropriate for cell growth and/or maintenance. Suitable cell culture conditions are well known in the art those of skill in the art appreciate that methods for culturing cells are known in the art and can and will vary depending on the cell type. Routine optimization may be used, in all cases, to determine the best techniques for a particular cell type.

Cells useful for the methods provided herein can be freshly isolated primary cells or obtained from a frozen aliquot of a primary cell culture. In some cases, cells are electroporated for uptake of gRNAs and the base editing fusion protein. As described in the Examples that follow, electroporation conditions for some assays (e.g., for T cells) can comprise 1400 volts, pulse width of 10 milliseconds, 3 pulses. Following electroporation, electroporated T cells are allowed to recover in a cell culture medium and then cultured in a T cell expansion medium. In some cases, electroporated cells are allowed to recover in the cell culture medium for about 5 to about 30 minutes (e.g., about 5, 10, 15, 20, 25, 30 minutes). Preferably, the recovery cell culture medium is free of an antibiotic or other selection agent. In some cases, the T cell expansion medium is complete CTS OpTmizer T-cell Expansion medium.

Applications

The RNA scaffolds of the invention can be used for the following applications genome editing, genome screening, generation of therapeutic cells, genome tagging, epigenome editing, karyotype engineering, chromatin imaging, transcriptome and metabolic pathway engineering, genetic circuits engineering, cell signalling sensing, cellular events recording, lineage information reconstruction, gene drive, DNA genotyping, miRNA quantification, in vivo cloning, site-directed mutagenesis, genomic diversification, and proteomic analysis in situ.

Applications also include research of human diseases such as cancer immunotherapy, antiviral therapy, bacteriophage therapy, cancer diagnosis, pathogen screening, microbiota remodelling, stem-cell reprogramming, immunogenomic engineering, vaccine development, and antibody production.

### Definition

A nucleic acid or polynucleotide refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded. A person skilled in the art would understand that uracil is a nucleotide which replaces thymine in the RNA format. DNA sequences as disclosed herein will have a thymine nucleotide and the corresponding RNA sequences will have a uracil nucleotide at the same position.

The term “isolated” when referring to nucleic acid molecules or polypeptides means that the nucleic acid molecule or the polypeptide is substantially free from at least one other component with which it is associated or found together in nature.

As used herein, the term “guide RNA” generally refers to an RNA molecule (or a group of RNA molecules collectively) that can bind to a CRISPR protein and target the CRISPR protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to or modify using a CRISPR system. A target sequence may be within a nucleic acid in vitro or in vivo within the genome of a cell, which may be any form of single-stranded or double-stranded nucleic acid.

A “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas9 polynucleotides are derived from the wild type Cas9 protein amino acid sequence. Also, the variant mammalian codon-optimized Cas9 polynucleotides, including the Cas9 single mutant nickase (nCas9, such as nCas9D10A) and Cas9 double mutant null-nuclease (dCas9, such as dCas9 D10A H840A), are derived from the polynucleotide encoding the wild type mammalian codon-optimized Cas9 protein.

As used herein the term “wild type” is a term of the art understood by skilled persons and means the typical form of an organism, strain, gene or characteristic as it occurs in nature as distinguished from mutant or variant forms.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. For example, the mutant forms of mammalian codon-optimized Cas9 (hspCas9), including the Cas9 single mutant nickase and the Cas9 double mutant null-nuclease, are variants of the mammalian codon-optimized wild type Cas9 (hspCas9). The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 96%, 97%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants include polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule, for example, mutant variants of the Cas9 polypeptide that have modified or lost nuclease activity. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where a substitution within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine. Amino acids having aromatic side chains include: phenylalanine, tyrosine and tryptophan. Amino acids having positively charged side chains include: lysine, arginine and histidine. Amino acids having negatively charged side chains include: aspartate and glutamate.

A “Cas9 mutant” or “Cas9 variant” refers to a protein or polypeptide derivative of the wild type Cas9 protein such as S. pyogenes Cas9 protein, e.g., a protein having one or more point mutations, insertions, deletions, truncations, a fusion protein, or a combination thereof. It retains substantially the RNA targeting activity of the Cas9 protein. The protein or polypeptide can comprise, consist of, or consist essentially of a fragment of S. pyogenes Cas9 protein. In general, the mutant/variant is at least 50% (e.g., any number between 50% and 100%, inclusive) identical to S. pyogenes Cas9 protein. The mutant/variant can bind to an RNA molecule and be targeted to a specific DNA sequence via the RNA molecule, and may additional have a nuclease activity. Examples of these domains include RuvC like motifs (aa. 7-22, 759-766 and 982-989 of the S. pyogenes Cas9 protein) and HNH motif (aa 837-863). See Gasiunas et al., Proc Natl Acad Sci USA. 2012 Sep. 25; 109(39): E2579-E2586 and WO2013176772.

“Complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part I, Second Chapter “Overview of principles of hybridization and the strategy of nucleic acid probe assay”, Elsevier, N.Y.

“Hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytidine and guanine (C and G), other base pairs may form (e.g., Adams et al., The Biochemistry of the Nucleic Acids, 11th ed., 1992).

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bonds formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The terms “subject” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed. In some embodiments, a subject may be an invertebrate animal, for example, an insect or a nematode; while in others, a subject may be a plant or a fungus.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “contacting,” when used in reference to any set of components, includes any process whereby the components to be contacted are mixed into same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components, such as an Cas protein or guide RNA, includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

Various exemplary embodiments of compositions and methods according to this invention are now described in the following Examples.

### EXAMPLES

### Example 1—Modifications to the RNA Scaffold

sgRNA Sequence Design

A complete list of sgRNA designs used and their sequences are displayed in table 4. All sgRNA designs are based on the S. pyrogenes sgRNA consisting of a target specific 20 nt spacer sequence, a 76 nt b constant region sgRNA sequence, and a 7 nt poly-T U6 termination signal. All modifications were made to the constant component of the sgRNA and consist of the inclusion of the RNA aptamer hairpins and/or the extension of the repeat:anti-repeat of the stem. A single copy (1×MS2) or 2 copies (2×MS2) of the MS2 hairpin sequence (C5 variant) were incorporated into either the tetra-loop, stem-loop2 or the 3′ of the sgRNA. For 2×MS2 tracrRNAs two designs were pursued with one integrating 2 copies of the C5 MS2 variant into the 3′ of the sgRNA, and a second design consisting of the C5 variant positioned at stem-loop2 and the engineered MCP protein binding f6 aptamer assimilated into the 3′ of the sgRNA. The f6 aptamer is a different variant used for 2×MS2 plasmid design. Various extensions of the upper-stem of the repeat:anti-repeat were incorporated either side of the stem and in each case the extension incorporates the native S pyrogenes sequence.

TABLE 4
Design and sequences of different sgRNAs. N denotes the 20 nt target specific
spacer sequence. The constant sgRNA sequence as previously described is highlighted
in bold, the extended repeat: anti-repeat sequences are underlined. The MS2 (C5
variant) or f6 aptamer sequences are displayed in italics whilst the extensions
to the aptamer and linkers sequences are shown in in italics and underlined. US =
upper-stem of the repeat: antirepeat; TL = tetraloop; SL2 = stem-loop 2.

| sgRNA name | sgRNA sequence |
|---|---|
| S. pyrogenes | NNNNNNNNNNNNNNNNNNNNGTTTTAGAGCTAGAAATAGCAAGTTA |
| sgRNA (SEQ ID | AAATAAGGCTAGTCCGTTATCAACTTGAAAAAGTGGCACCGAGTC |
| NO: 40) | GGTGCTTTTTTT |
| 2xMS2_3′ (SEQ | NNNNNNNNNNNNNNNNNNNNGTTTTAGAGCTAGAAATAGCAAGTTA |
| ID NO: 41) | AAATAAGGCTAGTCCGTTATCAACTTGAAAAAGTGGCACCGAGTC |
| | GGTGCGGGAGCACATGAGGATCACCCATGTGCCACGAGCGACATGAGGA |
| | TCACCCATGTCGCTCGTGTTCCCTTTTTTT |
| 1xMS2-TL (SEQ | NNNNNNNNNNNNNNNNNNNNGTTTTAGAGCTAGGCCAACATGAGGAT |
| ID NO: 42) | CACCCATGTCTGCAGGGCCTAGCAAGTTAAAATAAGGCTAGTCCGTT |
| | ATCAACTTGAAAAAGTGGCACCGAGTCGGTGCTTTTTTT |
| 1xMS2_3′ (SEQ | NNNNNNNNNNNNNNNNNNNNGTTTTAGAGCTAGAAATAGCAAGTTA |
| ID NO: 43) | AAATAAGGCTAGTCCGTTATCAACTTGAAAAAGTGGCACCGAGTC |
| | GGTGCGCGCACATGAGGATCACCCATGTGCTTTTTTT |
| 7 bp | NNNNNNNNNNNNNNNNNNNNGTTTTAGAGCTATGCTGTTGAAAAAC |
| Extended_US | AGCATAGCAAGTTAAAATAAGGCTAGTCCGTTATCAACTTGAAAAA |
| (SEQ ID NO: 44) | GTGGCACCGAGTCGGTGCTTTTTTT |
| 2xMS2_3′ | NNNNNNNNNNNNNNNNNNNNGTTTTAGAGCTATGCTGTTGAAAA |
| 7 bp_extended_US | ACAGCATAGCAAGTTAAAATAAGGCTAGTCCGTTATCAACTTGAAA |
| (SEQ ID NO: | AAGTGGCACCGAGTCGGTGCGGGAGCACATGAGGATCACCCATGTGC |
| 45) | CACGAGCGACATGAGGATCACCCATGTCGCTCGTGTTCCCTTTTTTT |
| 1xMS2-TL_7 bp | NNNNNNNNNNNNNNNNNNNNGTTTTAGAGCTATGCTGTTGGCCAA |
| extended_US | CATGAGGATCACCCATGTCTGCAGGGCCAACAGCATAGCAAGTTAAA |
| (SEQ ID NO: 46) | ATAAGGCTAGTCCGTTATCAACTTGAAAAAGTGGCACCGAGTCGG |
| | TGCTTTTTTT |
| 1xMS2-3′_2 bp | NNNNNNNNNNNNNNNNNNNNGTTTTAGAGCTATGGAAA |
| extended-_US | CATAGCAAGTTAAAATAAGGCTAGTCCGTTATCAACTTGAAAAAGT |
| (SEQ ID NO: 47) | GGCACCGAGTCGGTGCGCGCACATGAGGATCACCCATGTGCTTTTTTT |
| 1xMS2-3′_5 bp- | NNNNNNNNNNNNNNNNNNNNGTTTTAGAGCTATGCTGGAAA |
| extended_US | CAGCATAGCAAGTTAAAATAAGGCTAGTCCGTTATCAACTTGAAAA |
| (SEQ ID NO: 48) | AGTGGCACCGAGTCGGTGCGCGCACATGAGGATCACCCATGTGCTTTT |
| | TTT |
| 1xMS2-3′_7 bp- | NNNNNNNNNNNNNNNNNNNNGTTTTAGAGCTATGCTGTTGAA |
| extended_US | AAACAGCATAGCAAGTTAAAATAAGGCTAGTCCGTTATCAAC |
| (SEQ ID NO: 49) | TTGAAAAAGTGGCACCGAGTCGGTGCGCGCACATGAGGATCAC |
| | CCATGTGCTTTTTTT |
| 1xMS2-3′_10 bp- | NNNNNNNNNNNNNNNNNNNNGTTTTAGAGCTATGCTGTTTTGG |
| extended_US | AAACAAAACAGCATAGCAAGTTAAAATAAGGCTAGTCCGTTA |
| (SEQ ID NO: 50) | TCAACTTGAAAAAGTGGCACCGAGTCGGTGCGCGCACATGAG |
| | GATCACCCATGTGCTTTTTTT |
| 1xMS2- | NNNNNNNNNNNNNNNNNNNNGTTTTAGAGCTATGCTGTTGAA |
| SL2_7 bp- | AAACAGCATAGCAAGTTAAAATAAGGCTAGTCCGTTATCAAC |
| extended_US | TTGGCCAACATGAGGATCACCCATGTCTGCAGGGCCAAGTGGCAC |
| (SEQ ID NO: 51) | CGAGTCGGTGCTTTTTTT |
| 2xMS2_C5- | NNNNNNNNNNNNNNNNNNNNGTTTTAGAGCTATGCTGTTGAAAAA |
| SL2_f6-3′_7 bp | CAGCATAGCAAGTTAAAATAAGGCTAGTCCGTTATCAACTTGGCCA |
| extended-US | ACATGAGGATCACCCATGTCTGCAGGGCCAAGTGGCACCGAGTCGGTG |
| (SEQ ID NO: 52) | CGCGCCCACAGTCACTGGGGCTTTTTTT |

### Plasmid Design

Aside from the sgRNA, all components of the base-editing system were encoded on one vector and expressed as a single polycistronic unit from a CMV promoter. The vector encodes the expression of an APOBEC-1-MCP fusion protein and nCas9 (D10A) fused to UGI though its C-terminus—the nCas9-UGI fusion protein was flanked by 2 copies of the SV40 NLS at the C terminus of nCas9 and the N terminus of UGI. Additionally, the vector encodes the expression of turboRFP to allow the monitoring of transfection efficiency.

The sgRNA component of the base editing system was expressed on a separate vector with expression being driven by the RNA polymerase III U6 promoter. The sgRNA was expressed as a single unit encompassing the crRNA and tracrRNA components of S. pyrogenes Cas9 linked by an artificial tetra-loop as previously described. A list of sgRNA target sgRNA sequences are shown in table 5, if the target did not possess a 5′ G then a G was added as required for expression from a U6 promoter.

The design of the expression of the BE4max base editor was as described previously.

TABLE 5
sgRNA target site sequences for base editing. Cs that lie
within editing window are shown in bold.

| Target name | Target sequence |
|---|---|
| Site2 | GAAC¹AC²AAAGCATAGACTGC (SEQ ID NO: 53) |
| Site3 | GGC¹C²C³AGACTGAGCACGTGA (SEQ ID NO: 54) |
| CTNNB1 | CTGGAC²TC³TGGAATCCATTC (SEQ ID NO: 55) |
| EGFR | ATC¹AC²GCAGCTCATGCCCTT (SEQ ID NO: 56) |
| PCSK9 | CAGGTTC²C³ACGGGATGCTCT (SEQ ID NO: 57) |
| FANCF | GGAATC¹C²C³TTCTGCAGCACC (SEQ ID NO: 58) |
| TRAC | TTC¹GTATC²TGTAAAACCAAG (SEQ ID NO: 59) |
| B2M | CTTAC²C³C⁴C⁵ACTTAACTATCT (SEQ ID NO: 60) |
| CR0118_PDCD1 | CAGTTCCAAACCCTGGTGGT (SEQ ID NO: 61) |
| CR0107_PDCD1 | GGGGGTTCCAGGGCCTGTCT (SEQ ID NO: 62) |
| CR0057- | TTCGTATCTGTAAAACCAAG (SEQ ID NO: 63) |
| TRAC_EX3 | |
| CR0151_CD2 | GTTCAGCCAAAACCTCCCCA (SEQ ID NO: 64) |
| CR0121_PDCD1 | GGAGTCTGAGAGATGGAGAG (SEQ ID NO: 65) |
| CR0165_CIITA | CAGCTCACAGTGTGCCACCA (SEQ ID NO: 66) |
| TRAC_22550571 | TTCAAAACCTGTCAGTGATT (SEQ ID NO: 67) |
| PDCD1_241852953 | GGGGGTTCCAGGGCCTGTCT (SEQ ID NO: 68) |
| CTNNB1 | CTGGACTCTGGAATCCATTC (SEQ ID NO: 69) |

### Cell Culture and Transfection

HEK293 cells were cultured in DMEM (Dulbecco's modified Eagle medium) supplemented with 10% FBS. 24 hours prior to transfection 50,000 cells were seeded into a single well of a 24-well plate to achieve ˜70% confluency for transfection. After 24 hours the cells were lipid transfected with 200 ng of plasmid DNA (150 ng pin-point/BE4max vector and 50 ng sgRNA expression vector) using Lipofectamine 3000 reagent (ThermoFisher scientific).

### Cell Lysis and Flow Cytometry

Following 72 hours after transfection the medium was removed, and the cells were washed 1× with PBS and detached from the well with 100 μl of TrypLE express enzyme (ThermoFisher scientific). The dissociated cells were then centrifuged at 300×rpm for 5 minutes at room temperature and the supernatant was decanted. The pelleted cells were washed 1× in PBS and again centrifuged at 300×rpm for 5 minutes and the supernatant was discarded, after which the pelleted cells were resuspended in 100 ul of PBS. 20 μl of the resuspended cells were transferred to a 96 well plate and were incubated with 36 μl of DirectPCR lysis reagent (Viagen biotech) under the following conditions: 55° C. for 30 minutes followed by 95° C. for 30 minutes, the cell lysates were stored at −20° C. The remaining 80 μl of the resuspended cells were transferred to a 96 well plate and pelleted by centrifugation at 300×rpm for 5 minutes at room temperature. The supernatant was decanted, and cells were resuspended in 50 μl MACS buffer (Miltenyi Biotec) supplemented with 0.5% BSA ready for flow cytometry analysis. All flow cytometry was performed using the iQue3 (Sartorius).

### PCR Amplification of Targeted Regions

1 μl of cell lysate obtained using the DirectPCR lysis reagent was used per PCR reaction. The Q5 high-fidelity 2× master mix (NEB) was used for amplification of sgRNA target sites, reaction mixes were set up as follows:

| Reagent | volume |
|---|---|
| Q5 2x master mix | 12.5 μl |
| Forward primer (10 μM) | 1.25 μl |
| Reverse primer (10 μM) | 1.25 μl |
| Cell lysate | 1.0 μl |
| Nuclease-free water | 9.0 μl |
| Total | 25 μl |

The PCR reactions were performed under the following thermocycling conditions:

| Step | Temperature | Time |
|---|---|---|
| Initial denaturation | 98° C. | 30 seconds |
| 30 cycles | 98° C. | 10 seconds |
| | 64° C./68° C. | 30 seconds |
| | 72° C. | 30 seconds |
| Final extension | 72° C. | 2 minutes |

Primers used and their annealing temperatures are detailed in Table 6 below:

| | | | Annealing | |
|---|---|---|---|---|
| | Target | | Temperature/ | |
| Primer | Site | Sequence | ° C. | SEQ ID NO |
| Site 2 F | Site 2 | TGGCCCTTCAAGTTACTGCA | 68 | SEQ ID NO: 70 |
| Site 2 R | | AGCACATGACAGTTAAGGTTTGT | | SEQ ID NO: 71 |
| Site 3 F | Site 3 | AAACGCCCATGCAATTAGTC | 68 | SEQ ID NO: 72 |
| Site 3 R | | AGCCCCTGTCTAGGAAAAGC | | SEQ ID NO: 73 |
| CTNNB1 F | CTNNB1 | CAATGGGTCATATCACAGATTCTT | 64 | SEQ ID NO: 74 |
| CTNNB1 R | | CCAGCTACTTGTTCTTGAGTGAA | | SEQ ID NO: 75 |
| EGFR F | EGFR | TCATGCGTCTTCACCTGAA | 64 | SEQ ID NO: 76 |
| EGFR R | | CGCACACACATATCCCCATG | | SEQ ID NO: 77 |
| PCSK9 F | PCSK9 | CACTAGCAGGGACAAGGTGG | 68 | SEQ ID NO: 78 |
| PCSK9 R | | ATTCAGCTCAGATGGGGTGG | | SEQ ID NO: 79 |
| FANCF F | FANCF | CGCTGGGAGATTGACATGCA | 68 | SEQ ID NO: 80 |
| FANCF R | | CTCTTGCCTCCACTGGTTGT | | SEQ ID NO: 81 |
| TRAC F | TRAC | ACCTACCCCATCCCCAGAAG | 68 | SEQ ID NO: 82 |
| TRAC R | | TCCCTAAACCCCACTCCCAG | | SEQ ID NO: 83 |
| B2M F | B2M | TGGGTTTCATCCATCCGACAT | 64 | SEQ ID NO: 84 |
| B2M R | | ATGGGATGGGACTCATTCAGG | | SEQ ID NO: 85 |

Unpurified PCR amplicons were subjected to Sanger sequencing by Genewiz.

### Example 2—Base Editing Efficiency of Modified RNA Scaffolds

### RNA Synthesis

All crRNA and tracrRNA were synthesized by Horizon Discovery using either 2′-acetoxy ethyl orthoester (2′-ACE) or 2′-tert-butyldimethylsilyl (2′-TBDMS) protection chemistries. Chemical modifications were included where noted, including two 2′-O-Methyl nucleotides and two phosphorothioate linkages (2×MS modification) at the 5′ end of the crRNA and 3′ end of the tracrRNA. RNA oligos were 2′-deprotected/desalted and purified by either high-performance liquid chromatography (HPLC) or polyacrylamide gel electrophoresis (PAGE). Oligos were resuspended in 10 mM Tris pH7.5 buffer prior to electroporation.

### Electroporation

HEK 293T cells (ATCC, #CRL-11268) were electroporated using the Invitrogen™ Neon™ Transfection System, 10 μL Kit. A mixture of 50,000 cells, 1 μg of mRNA, and 6 μM of synthetic crRNA:tracrRNA were electroporated at 1150V for 20 ms and for 2 pulses. mRNA (obtained from TriLink or in vitro transcribed in house by standard methods) was mixed at a 3:1 molar ratio of nCas9-UGI to MCP-AID or MCP-APOBEC. Cells were plated in a 96-well plate with full serum growth media and harvested after 72 hours for further processing.

### Cell Processing

Cells were lysed in 100 μL of a buffer containing proteinase K (Thermo Scientific, #FERE00492), RNase A (Thermo Scientific, #FEREN0531), and Phusion HF buffer (Thermo Scientific, #F-518L) for 30 min at 56° C., followed by a 5 minute heat inactivation at 95° C. This cell lysate was used to generate 200-400 nucleotide PCR amplicons spanning the region containing the base editing site(s). Unpurified PCR amplicons were subjected to Sanger sequencing by Genewiz.

### Editing Analysis

Base editing efficiencies were calculated from the AB1 files using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 20 bp input guide sequence.

### Example 3: Base Editing System Applied to Human Primary Immune Cells Utilising Lentiviral Integrated sgRNA

In this example, primary human Pan T lymphocytes were used to prove the utility of the base editing mRNA components in primary immune cells in the presence of a constitutive expression sgRNA with RNA aptamers under the control of a PolIII promoter. The Pan T cells were activated utilising anti-CD3 and anti-CD28 and then transduced using enriched and concentrated lentiviral particles. Successfully transduced cells were selected using puromycin selection to ensure >95% of the population had at least one copy of the lentiviral insert. During the selection the T cells were re-activated by anti-CD3 and anti-CD28, then the cells were electroporated with mRNA components for both the deaminase-MCP and the nCas9-UGI-UGI components. The cells were then incubated for a further 72-96 hours and cells were checked for surface KO by flow cytometry and the base editing was checked by targeted PCR amplification and Sanger sequencing.

### Example 4: Base Editing System Applied to Human Primary Immune Cells Utilising Synthetic crRNA and tracrRNA-Aptamer Guides

In this example, primary human Pan T lymphocytes were used to prove the utility of the base editing system with crRNA and aptamer modified tracrRNA components in primary immune cells. The Pan T cells were activated utilising anti-CD3 and anti-CD28 and then cells were electroporated with mRNA components for both the deaminase-MCP, nCas9-UGI-UGI components, tracrRNA-Aptamer and the crRNA. The cells were then incubated for a further 72-96 hours and cells were checked for surface KO by flow cytometry and the base editing was checked by targeted PCR amplification and Sanger sequencing.

The data show the base editing system can edit primary immune cells, without the necessity to integrate DNA into the genome (via lentiviral cassettes), utilising varied crRNA and tracrRNA-Aptamer with mRNA components. The results display a distinct RNA aptamer and deaminase specificity with the Apobec1 having preferences for the single RNA motif, whilst the AID deaminase preferring the double RNA motif in this context. The results display a high utility of the base editing system for altering specific bases for function protein knock-out by surface staining and flow cytometry and by alterations at the DNA level.

### Material and Methods

### Guides

Internally generated data was used to specify base editing windows calculated at set distances from the PAM motif (NGG). The data was used to development algorithms to predict Phenotype or Gene KO applicable guides sequence for the following genes: TRAC, TRBC1, TRBC2, PDCD-1, B2M, and CD52 (Table 7). The crRNAs and tracrRNA were synthesised by Horizon Discovery (formerly Dharmacon) and Agilent.

Synthetic crRNA Sequence (SEQ ID NO: 86):
mN*mN*NNNNNNNNNNNNNNNNNNGUUUUAGAGCUAUGCUGUUUUG
2′OMe (m) and phosphorothioate (*) modified residues
Synthetic 1xMS2 tracrRNA-Aptamer Sequence (SEQ ID NO: 87):
AACAGCAUAGCAAGUUAAAAUAAGGCUAGUCCGUUAUCAACUUGAAAAAGUGGCACCGA
GUCGGUGCGCGCACAUGAGGAUCACCCAUGUGCUUUUmU*mU*U
2′OMe (m) and phosphorothioate (*) modified
Synthetic 2xMS2 tracrRNA-Aptamer Sequence (SEQ ID NO: 88):
AACAGCAUAGCAAGUUAAAAUAAGGCUAGUCCGUUAUCAACUUGAAAAAGUGGCACCGA
GUCGGUGCGGGAGCACAUGAGGAUCACCCAUGUGCCACGAGCGACAUGAGGAUCACCCA
UGUCGCUCGUGUUCCCUUUUmU*mU*U
2′OMe (m) and phosphorothioate (*) modified residues
Lentiviral sgRNA sequences (SEQ ID NO: 89):
NNNNNNNNNNNNNNNNNNNNGUUUUAGAGCUAGAAAUAGCAAGUUAAAAUAAGGCUAG
UCCGUUAUCAACUUGAAAAAGUGGCACCGAGUCGGUGCgggagcACAUGAGGAUCACCCAU
GUgccacgagcgACAUGAGGAUCACCCAUGUcgcUcgUgUUcccUUUU

mRNA Component Generation

Messenger RNA molecules were custom generated by Trilink utilising modified nucleotides: Pseudouridine and 5-Methyl-Cytosine. The mRNA components translated to the following proteins: Deaminase AID=NLS-hAID-Linker-MCP, Deaminase Apobec1=NLS-rApobec1-Linker-MCP, and Cas9-UGI-UGI=NLS-nCas9-UGI-UGI-NLS

### Plasmid Construction

The lentiviral construct included additional selectable markers (e.g. antibiotics, fluorescent proteins) to ensure that single integration copies were present within the genome of the target cell population. Sequences for the specific guides sequences were cloned (by T4 DNA ligase technology) into overhangs generated by Type IIS restriction enzyme sites. The target construct ensured the guide sequence was perfectly in frame for efficient transcription from the human U6 PolIII promoter (inclusion of a G nucleotide if not at the 5′ of the sequence) and to extended into the Cas9 scaffold and aptamer sequences before termination sequences. Plasmid clones were check by Sanger sequencing and restriction digestion QC, before being expanded for large-scale plasmid preparation (e.g. maxiprep).

### Lentiviral Particle Generation

sgRNA-Aptamer lentiviral constructs were made in functional lentiviral particles using 3^{rd} generation plasmid systems (Horizon Discovery). Viral particles were then concentrated by diafiltration and aliquoted for transduction.

### Lentiviral Transduction

T cells were activated for >48 hours and transduced with a MOI of 0.1 by Retronectin (T100B, Takara-bio) treated plates and incubation at 37 C and 5% CO2 overnight.

### Frozen T Cells Culturing

Sources of frozen CD3+ T Cells (Hemacare) were thawed and then cultured into Immunocult XT media (STEMCELL Technologies) with 1× Penicillin/Streptomycin (Thermofisher) at 37 C and 5% CO2.

### T Cell Electroporation

After 48-72 post-activation T cells were electroporated with using the Neon Electroporator (Thermofisher) or 4D Nucleofector (Lonza). Neon Electroporator conditions were 1600 v/10 ms/3 pulses with a 10 ul tip with 250 k cells, combined total of mRNA amount of 1-5 ug, for both the Deaminase-MCP and nCas9-UGI-UGI, and where applicable 0.2-1.8 umol of complexed crRNA:tracrR or sgRNA. 4D Nucleofector conditions were EO-115 with a 20 ul cuvette with 500 k combined total of mRNA amount of 1-5 ug, for both the Deaminase-MCP and nCas9-UGI-UGI (synthesised by Trilink), and 0.2-1.8 umol of complexed crRNA:tracrR or sgRNA (Horizon Discovery). Post-electroporation cells were transferred to Immunocult XT media with 100 U IL-2, 100 U IL-7 and 100 U IL-15 (STEMCELL Technologies) and cultured at 37 C and 5% CO2 for 48-72 hours.

### CD3+ T Cell Activation

T cells were activated by using 1:1 bead:cell ratio of Dynabeads Human T Activator CD3/CD28 beads (Thermofisher) cultured in Immunocult XT media (STEMCELL Technologies) in the presence of 100 U/ml IL-2 (STEMCELL Technologies) and 1× Penicillin/Streptomycin (Thermofisher) at 37 C and 5% CO2 for 48 hours. Post-activation, beads were removed by placement on a magnet and the transfer of the cells back into culture.

### Flow Cytometry

T cell identity and QC was confirmed by CD3-antibody staining (Biolegend). T cell activation confirmed by CD25 staining. Phenotypic Gene KO: TRAC was confirmed by CD3 and TCRab antibody staining (Biolegend) and B2M by B2M-Antibody (Biolegend); any phenotype data was the percentage change against reference material on viable cells only as ascertained by DAPI staining (BD Bioscience).

### Genomic DNA Analysis

Genomic DNA was released from lysed cells 48-72 hours post-electroporation. Loci of interest were amplified by PCR and products then sent for Sanger sequencing (Genewiz). Data was analysed by proprietary in-house software.

TABLE 7
Single-guide RNAs (sgRNAs) for TRAC, TRBC1, TRBC2, PDCD1, CD52 and B2M
Functional Knock-Out Base Editing

| Gene Name | Guide ID | KO Type* | Strand | Guide Sequence | PAM |
|---|---|---|---|---|---|
| B2M | B2M_1 | Stop | sense | CACAGCCCAAGATAGTTAAG | TGG |
| | | | | (SEQ ID NO: 90) | |
| | B2M_2 | Stop | sense | ACAGCCCAAGATAGTTAAGT | GGG |
| | | | | (SEQ ID NO: 91) | |
| | B2M_3 | Stop | anti | TTACCCCACTTAACTATCTT | GGG |
| | | | | (SEQ ID NO: 92) | |
| | B2M_4 | Stop | anti | CTTACCCCACTTAACTATCT | TGG |
| | | | | (SEQ ID NO: 93) | |
| | B2M_5 | Splice | anti | ACTCACGCTGGATAGCCTCC | AGG |
| | | | | (SEQ ID NO: 94) | |
| | B2M_6 | Splice | anti | TTGGAGTACCTGAGGAATAT | CGG |
| | | | | (SEQ ID NO: 95) | |
| | B2M_7 | Splice | anti | TCGATCTATGAAAAAGACAG | TGG |
| | | | | (SEQ ID NO: 96) | |
| | B2M_8 | Splice | anti | AACCTGAAAAGAAAAGAAAA | AGG |
| | | | | (SEQ ID NO: 97) | |
| CD52 | CD52_1 | Stop | sense | GTACAGGTAAGAGCAACGCC | TGG |
| | | | | (SEQ ID NO: 98) | |
| | CD52_2 | Stop | sense | CTCCTCCTACAGATACAAAC | TGG |
| | | | | (SEQ ID NO: 99) | |
| | CD52_3 | Stop | sense | CAGATACAAACTGGACTCTC | AGG |
| | | | | (SEQ ID NO: 100) | |
| | CD52_4 | Splice | anti | CTCTTACCTGTACCATAACC | AGG |
| | | | | (SEQ ID NO: 101) | |
| | CD52_5 | Splice | anti | GTATCTGTAGGAGGAGAAGT | GGG |
| | | | | (SEQ ID NO: 102) | |
| | CD52_6 | Splice | anti | TGTATCTGTAGGAGGAGAAG | TGG |
| | | | | (SEQ ID NO: 103) | |
| | CD52_7 | Splice | anti | GTCCAGTTTGTATCTGTAGG | AGG |
| | | | | (SEQ ID NO: 104) | |
| TRAC | TRAC_1 | Stop | sense | AACAAATGTGTCACAAAGTA | AGG |
| | | | | (SEQ ID NO: 105) | |
| | TRAC_2 | Stop | sense | CTTCTTCCCCAGCCCAGGTA | AGG |
| | | | | (SEQ ID NO: 106) | |
| | TRAC_3 | Stop | sense | TTCTTCCCCAGCCCAGGTAA | GGG |
| | | | | (SEQ ID NO: 107) | |
| | TRAC_4 | Stop | sense | AGCCCAGGTAAGGGCAGCTT | TGG |
| | | | | (SEQ ID NO: 108) | |
| | TRAC_5 | Stop | sense | TTTCAAAACCTGTCAGTGAT | TGG |
| | | | | (SEQ ID NO: 109) | |
| | TRAC_6 | Stop | sense | TTCAAAACCTGTCAGTGATT | GGG |
| | | | | (SEQ ID NO: 110) | |
| | TRAC_7 | Stop | sense | CCGAATCCTCCTCCTGAAAG | TGG |
| | | | | (SEQ ID NO: 111) | |
| | TRAC_8 | Splice | anti | CTTACCTGGGCTGGGGAAGA | AGG |
| | | | | (SEQ ID NO: 112) | |
| | TRAC_9 | Splice | anti | TTCGTATCTGTAAAACCAAG | AGG |
| | | | | (SEQ ID NO: 113) | |
| TRBC1/2 | TRBC1/2_1 | Stop | sense | CCACACCCAAAAGGCCACAC | TGG |
| | | | | (SEQ ID NO: 114) | |
| | TRBC1/2_2 | Stop | anti | CCCACCAGCTCAGCTCCACG | TGG |
| | | | | (SEQ ID NO: 115) | |
| | TRBC1/2_3 | Stop | sense | CGCTGTCAAGTCCAGTTCTA | CGG |
| | | | | (SEQ ID NO: 116) | |
| | TRBC1/2_4 | Stop | sense | GCTGTCAAGTCCAGTTCTAC | GGG |
| | | | | (SEQ ID NO: 117) | |
| | TRBC1/2_5 | Stop | sense | AGTCCAGTTCTACGGGCTCT | CGG |
| | | | | (SEQ ID NO: 118) | |
| | TRBC1/2_6 | Stop | sense | CACCCAGATCGTCAGCGCCG | AGG |
| | | | | (SEQ ID NO: 119) | |
| | TRBC1/2_7 | Splice | anti | ACCTGCTCTACCCCAGGCCT | CGG |
| | | | | (SEQ ID NO: 120) | |
| | TRBC1/2_8 | Splice | anti | CCACTCACCTGCTCTACCCC | AGG |
| | | | | (SEQ ID NO: 121) | |
| TRBC1 | TRBC1_1 | Stop | sense | CACGGACCCGCAGCCCCTCA | AGG |
| | | | | (SEQ ID NO: 122) | |
| | TRBC1_2 | Stop | anti | GCGGGGGTTCTGCCAGAAGG | TGG |
| | | | | (SEQ ID NO: 123) | |
| | TRBC1_3 | Stop | anti | GTTGCGGGGGTTCTGCCAGA | AGG |
| | | | | (SEQ ID NO: 124) | |
| | TRBC1_4 | Stop | sense | ATGACGAGTGGACCCAGGAT | AGG |
| | | | | (SEQ ID NO: 125) | |
| | TRBC1_5 | Stop | sense | TGACGAGTGGACCCAGGATA | GGG |
| | | | | (SEQ ID NO: 126) | |
| | TRBC1_6 | Stop | anti | ACCTGCTCTACCCCAGGCCT | CGG |
| | | | | (SEQ ID NO: 127) | |
| | TRBC1_7 | Stop | sense | CCAACAGTGTCCTACCAGCA | AGG |
| | | | | (SEQ ID NO: 128) | |
| | TRBC1_8 | Stop | sense | CAACAGTGTCCTACCAGCAA | GGG |
| | | | | (SEQ ID NO: 129) | |
| | TRBC1_9 | Stop | sense | AACAGTGTCCTACCAGCAAG | GGG |
| | | | | (SEQ ID NO: 130) | |
| | TRBC1_10 | Splice | anti | GTCTGAAAGAAAGCAGGGAG | AGG |
| | | | | (SEQ ID NO: 131) | |
| | TRBC1_11 | Splice | anti | CCACAGTCTGAAAGAAAGCA | GGG |
| | | | | (SEQ ID NO: 132) | |
| | TRBC1_12 | Splice | anti | GCCACAGTCTGAAAGAAAGC | AGG |
| | | | | (SEQ ID NO: 133) | |
| | TRBC1_13 | Splice | anti | GACACTGTTGGCACGGAGGA | AGG |
| | | | | (SEQ ID NO: 134) | |
| | TRBC1_14 | Splice | anti | GTAGGACACTGTTGGCACGG | AGG |
| | | | | (SEQ ID NO: 135) | |
| | TRBC1_15 | Splice | anti | TACCATGGCCATCAACACAA | GGG |
| | | | | (SEQ ID NO: 136) | |
| | TRBC1_16 | Splice | anti | TTACCATGGCCATCAACACA | AGG |
| | | | | (SEQ ID NO: 137) | |
| TRBC2 | TRBC2_1 | Stop | anti | CCAGCTCAGCTCCACGTGGT | CGG |
| | | | | (SEQ ID NO: 138) | |
| | TRBC2_2 | Stop | sense | CACAGACCCGCAGCCCCTCA | AGG |
| | | | | (SEQ ID NO: 139) | |
| | TRBC2_3 | Stop | anti | GCGGGGGTTCTGCCAGAAGG | TGG |
| | | | | (SEQ ID NO: 140) | |
| | TRBC2_4 | Stop | anti | GTTGCGGGGGTTCTGCCAGA | AGG |
| | | | | (SEQ ID NO: 141) | |
| | TRBC2_5 | Stop | sense | ATGACGAGTGGACCCAGGAT | AGG |
| | | | | (SEQ ID NO: 142) | |
| | TRBC2_6 | Stop | sense | TGACGAGTGGACCCAGGATA | GGG |
| | | | | (SEQ ID NO: 143) | |
| | TRBC2_7 | Stop | anti | ACCTGCTCTACCCCAGGCCT | CGG |
| | | | | (SEQ ID NO: 144) | |
| | TRBC2_8 | Stop | sense | TCAACAGAGTCTTACCAGCA | AGG |
| | | | | (SEQ ID NO: 145) | |
| | TRBC2_9 | Stop | sense | CAACAGAGTCTTACCAGCAA | GGG |
| | | | | (SEQ ID NO: 146) | |
| | TRBC2_10 | Stop | sense | AACAGAGTCTTACCAGCAAG | GGG |
| | | | | (SEQ ID NO: 147) | |
| | TRBC2_11 | Splice | anti | CACAGTCTGAAAGAAAACAG | AGG |
| | | | | (SEQ ID NO: 148) | |
| | TRBC2_12 | Splice | anti | CCACAGTCTGAAAGAAAACA | AGG |
| | | | | (SEQ ID NO: 149) | |
| | TRBC2_13 | Splice | anti | GCCACAGTCTGAAAGAAAAC | AGG |
| | | | | (SEQ ID NO: 150) | |
| PDCD1 | PDCD1_1 | Stop | sense | TCCAGGCATGCAGATCCCAC | AGG |
| | | | | (SEQ ID NO: 151) | |
| | PDCD1_2 | Stop | sense | TGCAGATCCCACAGGCGCCC | TGG |
| | | | | (SEQ ID NO: 152) | |
| | PDCD1_3 | Stop | anti | CGACTGGCCAGGGCGCCTGT | GGG |
| | | | | (SEQ ID NO: 153) | |
| | PDCD1_4 | Stop | anti | ACGACTGGCCAGGGCGCCTG | TGG |
| | | | | (SEQ ID NO: 154) | |
| | PDCD1_5 | Stop | anti | ACCGCCCAGACGACTGGCCA | GGG |
| | | | | (SEQ ID NO: 155) | |
| | PDCD1_6 | Stop | anti | CACCGCCCAGACGACTGGCC | AGG |
| | | | | (SEQ ID NO: 156) | |
| | PDCD1_7 | Stop | anti | TGTAGCACCGCCCAGACGAC | TGG |
| | | | | (SEQ ID NO: 157) | |
| | PDCD1_8 | Stop | sense | GGGCGGTGCTACAACTGGGC | TGG |
| | | | | (SEQ ID NO: 158) | |
| | PDCD1_9 | Stop | sense | CGGTGCTACAACTGGGCTGG | CGG |
| | | | | (SEQ ID NO: 159) | |
| | PDCD1_10 | Stop | sense | CTACAACTGGGCTGGCGGCC | AGG |
| | | | | (SEQ ID NO: 160) | |
| | PDCD1_11 | Stop | anti | CACCTACCTAAGAACCATCC | TGG |
| | | | | (SEQ ID NO: 161) | |
| | PDCD1_12 | Stop | anti | GGGGTTCCAGGGCCTGTCTG | GGG |
| | | | | (SEQ ID NO: 162) | |
| | PDCD1_13 | Stop | anti | GGGGGTTCCAGGGCCTGTCT | GGG |
| | | | | (SEQ ID NO: 163) | |
| | PDCD1_14 | Stop | anti | GGGGGGTTCCAGGGCCTGTC | TGG |
| | | | | (SEQ ID NO: 164) | |
| | PDCD1_15 | Stop | sense | CAGCAACCAGACGGACAAGC | TGG |
| | | | | (SEQ ID NO: 165) | |
| | PDCD1_16 | Stop | sense | CCCGAGGACCGCAGCCAGCC | CGG |
| | | | | (SEQ ID NO: 166) | |
| | PDCD1_17 | Stop | sense | GGACCGCAGCCAGCCCGGCC | AGG |
| | | | | (SEQ ID NO: 167) | |
| | PDCD1_18 | Stop | sense | CGTGTCACACAACTGCCCAA | CGG |
| | | | | (SEQ ID NO: 168) | |
| | PDCD1_19 | Stop | sense | GTGTCACACAACTGCCCAAC | GGG |
| | | | | (SEQ ID NO: 169) | |
| | PDCD1_20 | Stop | sense | CGCAGATCAAAGAGAGCCTG | CGG |
| | | | | (SEQ ID NO: 170) | |
| | PDCD1_21 | Stop | sense | GCAGATCAAAGAGAGCCTGC | GGG |
| | | | | (SEQ ID NO: 171) | |
| | PDCD1_22 | Stop | sense | AGCCGGCCAGTTCCAAACCC | TGG |
| | | | | (SEQ ID NO: 172) | |
| | PDCD1_23 | Stop | sense | CGGCCAGTTCCAAACCCTGG | TGG |
| | | | | (SEQ ID NO: 173) | |
| | PDCD1_24 | Stop | sense | CAGTTCCAAACCCTGGTGGT | TGG |
| | | | | (SEQ ID NO: 174) | |
| | PDCD1_25 | Stop | anti | GGACCCAGACTAGCAGCACC | AGG |
| | | | | (SEQ ID NO: 175) | |
| | PDCD1_26 | Splice | anti | CACCTACCTAAGAACCATCC | TGG |
| | | | | (SEQ ID NO: 176) | |
| | PDCD1_27 | Splice | anti | GGAGTCTGAGAGATGGAGAG | AGG |
| | | | | (SEQ ID NO: 177) | |
| | PDCD1_28 | Splice | anti | TCTGGAAGGGCACAAAGGTC | AGG |
| | | | | (SEQ ID NO: 178) | |
| | PDCD1_29 | Splice | anti | TTCTCTCTGGAAGGGCACAA | AGG |
| | | | | (SEQ ID NO: 179) | |
| | PDCDl_30 | Splice | anti | TGACGTTACCTCGTGCGGCC | CGG |
| | | | | (SEQ ID NO: 180) | |
| | PDCD1_31 | Splice | anti | TCCCTGCAGAGAAACACACT | TGG |
| | | | | (SEQ ID NO: 181) | |
| | PDCD1_32 | Splice | anti | GAGACTCACCAGGGGCTGGC | CGG |
| | | | | (SEQ ID NO: 182) | |
| | PDCD1_33 | Splice | anti | TCTTTGAGGAGAAAGGGAGA | GGG |
| | | | | (SEQ ID NO: 183) | |
| | PDCD1_34 | Splice | anti | TTCTTTGAGGAGAAAGGGAG | AGG |
| | | | | (SEQ ID NO: 184) | |

*Stop = Premature stop codon, Splice = Splice site disruption

An example list of guides designs for both sgRNA and crRNA formats that can create a functional knock-out using the base editing technology exemplified. The list includes guides specific to the introduction of a premature stop codon and splice disruption sites, which were generated by in-house proprietary software.

### Example 5: Base Editing Efficiency of Modified RNA Scaffolds in crRNA:tracrRNA and sgRNAs

### RNA Synthesis

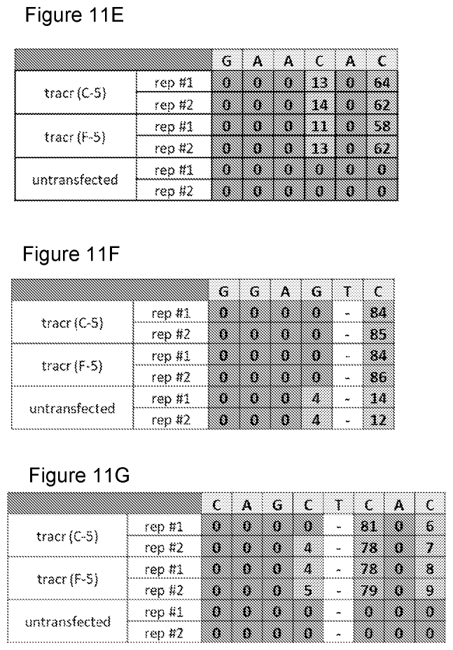

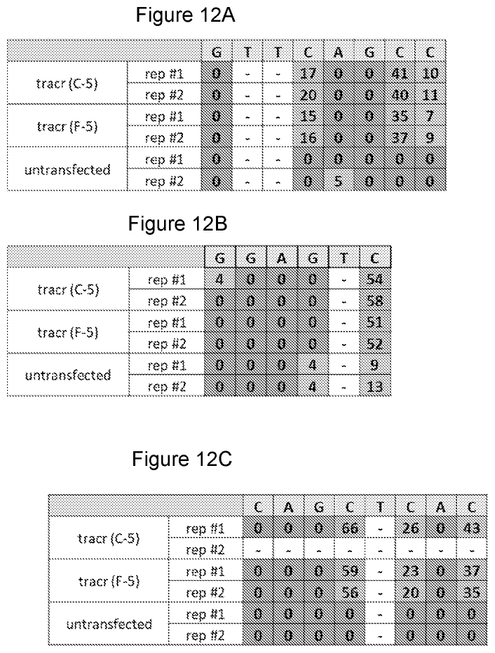

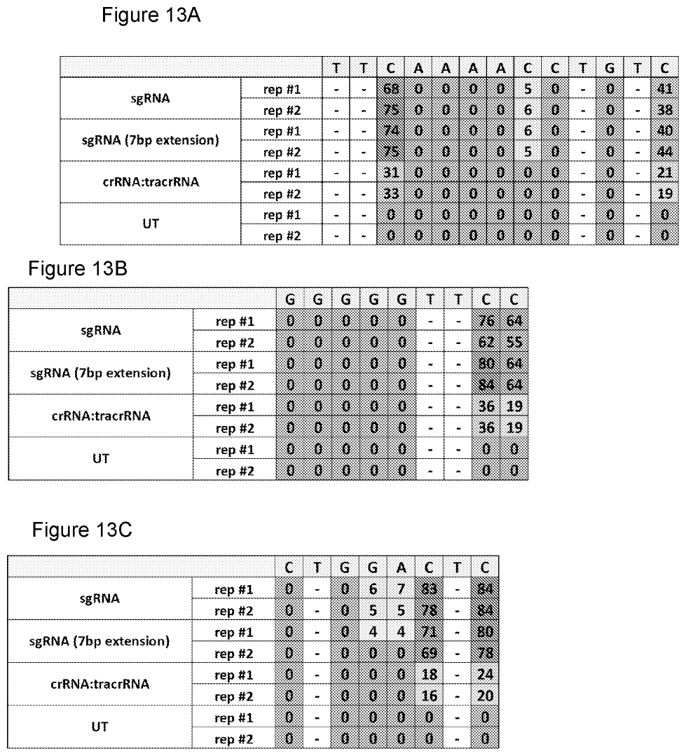

All crRNA, tracrRNA, and sgRNA were synthesized using either 2′-acetoxy ethyl orthoester (2′-ACE) or 2′-tert-butyldimethylsilyl (2′-TBDMS) protection chemistries. Chemical modifications were included as noted, including two 2′-O-Methyl nucleotides and two phosphorothioate (2×MS modifications) at the 5′ end of the crRNA and 3′ end of the tracrRNA. 2′ OMe is denoted as m and phosphorothioate is denoted as *. RNA oligos were 2′-deprotected/desalted and purified by either high-performance liquid chromatography (HPLC) or polyacrylamide gel electrophoresis (PAGE). Oligos were resuspended in 10 mM Tris pH7.5 buffer prior to transfection. Gene sites targeted by each cRNA are (A) CR0118_PDCD1, (B) CR0107_PDCD1, (C) CR0057-TRAC_EX3, (D) CR0151_CD2, (E) Site 2, (F) CR0121_PDCD1, and (G) CR0165_CIITA as shown in FIG. 11A-G, A) CR0151_CD2, (B) CR0121_PDCD1, and (C) CR0165_CIITA as shown in FIG. 12A-C, and (A) TRAC_22550571, (B) PDCD1_241852953, and (C) CTNNB1 as shown in FIG. 13A-C. sgRNA target site sequences for base editing are listed in Table 5.

### Transfection

U2OS nCas9 stably-transfected cells were transfected with DharmaFECT Duo, 25 nM synthetic crRNA:tracrRNA, and 200 ng of (a) rAPOBEC or (b) hAID mRNA. Cells were harvested at 72 hrs.

### Cell Processing

Cells were lysed in 100 μL of a buffer containing proteinase K (Thermo Scientific, #FEREO0492), RNase A (Thermo Scientific, #FEREN0531), and Phusion HF buffer (Thermo Scientific, #F-518L) for 30 min at 56° C., followed by a 5 minute heat inactivation at 95° C. This cell lysate was used to generate 200-400 nucleotide PCR amplicons spanning the region containing the base editing site(s). Unpurified PCR amplicons were subjected to Sanger sequencing by Genewiz.

### Editing Analysis

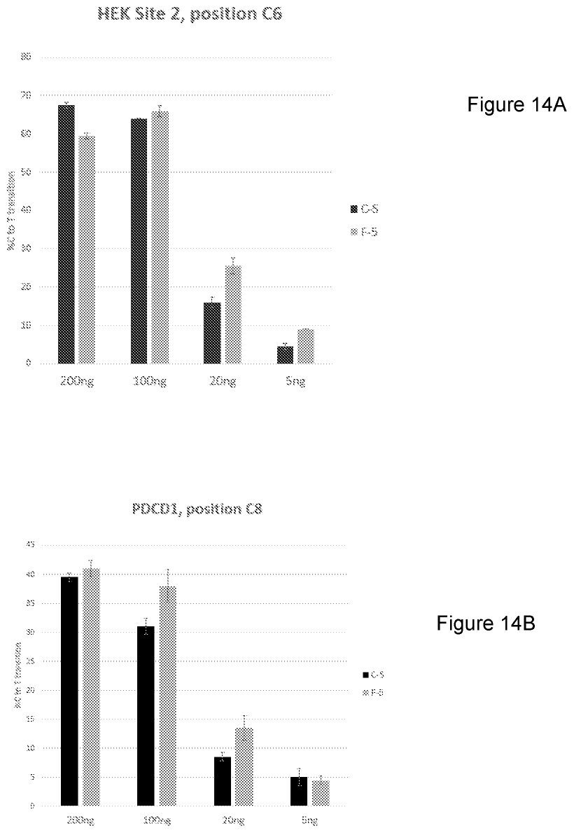

Base editing efficiencies were calculated from the AB1 files using the Chimera analysis tool, an adaptation of the open source tool BEAT. Chimera determines editing efficiency by first subtracting the background noise to define the expected variability in a sample. This allows the estimation of editing efficiency without the need to normalize to control samples. Following this, Chimera filters out any outliers from the noise using the Median Absolute Deviation (MAD) method and then assesses the editing efficiency of the base editor over the span of the 20 bp input guide sequence. FIG. 11 shows the comparative editing efficiency of the base editing system incorporating a single copy of either the C-5 or F-5 MS2 variants at the 3′ terminus of the tracrRNA. Data is shown for the following crRNAs; a) (A) CR0118_PDCD1, (B) CR0107_PDCD1, (C) CR0057-TRAC_EX3, (D) CR0151_CD2, (E) Site 2, (F) CR0121_PDCD1, and (G) CR0165_CIITA. The percentage of C to T editing detected indicates that the C-5 and F-5 variants offer comparable levels of base editing at all loci investigated, additionally the window of editing is equivocal between the two MS2 variants. FIG. 12 shows comparative editing efficiency of the base editing system incorporating a single copy of either the C-5 or F-5 MS2 variants at the 3′ terminus of the tracrRNA for the following crRNAs: (A) CR0151_CD2, (B) CR0121_PDCD1, and (C) CR0165_CIITA. FIG. 13 shows the level of base editing with chemically synthesized 1×MS2_3′ sgRNAs (C-5), or 1×MS2_3′_7 bp-extended_US sgRNAs (C-5) containing a 7-base pair extension of the repeat:anti-repeat upper stem. FIG. 14 demonstrates that when the amount of the MCP-deaminase is reduced down to 20 ng, the higher affinity F-5 MS2 tracrRNA results in a higher percentage of C to T editing compared to the C-5 MS2.

Synthetic 1×MS2 tracrRNA-Aptamer Sequences as Used in Example 5:

1×MS2_3′ tracrRNA (C-5) SEQ ID NO: 87 AACAGCAUAGCAAGUUAAAAUAAGGCUAGUCCGUUAUCAACUUGAAAAAGUGGCACCGA GUCGGUGCGCGCACAUGAGGAUCACCCAUGUGCUUUUmU*mU*U

1×MS2_3′ tracrRNA (F-5) SEQ ID NO: 185 AACAGCAUAGCAAGUUAAAAUAAGGCUAGUCCGUUAUCAACUUGAAAAAGUGGCACCGA GUCGGUGCGCGGCCCGG-2AdP-GGAUCACCACGGGCCUUUUmU*mU*U

2′OMe is denoted as m and phosphorothioate is denoted as *

Protein sequence of RNA scaffold mediated recruitment system (2×UGI):

SEQ ID NO: 186

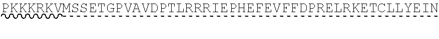

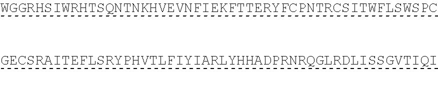

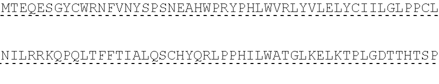

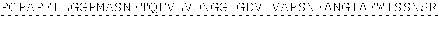

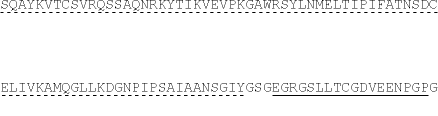

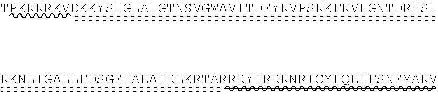

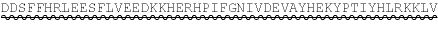

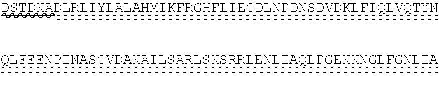

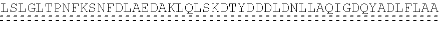

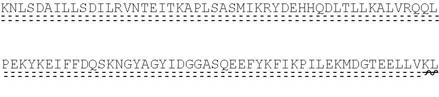

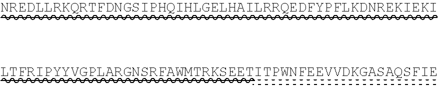

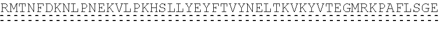

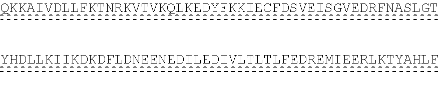

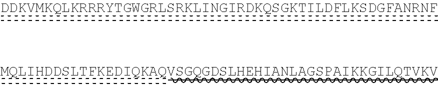

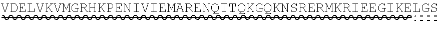

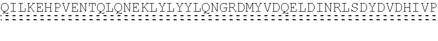

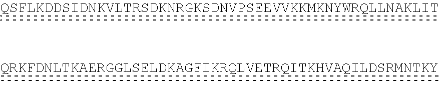

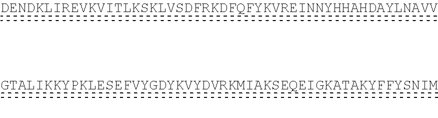

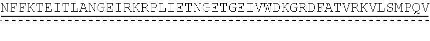

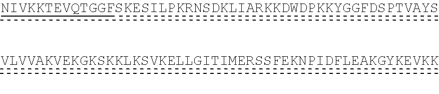

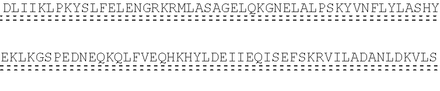

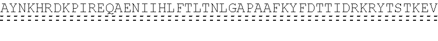

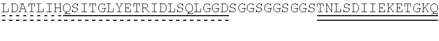

LVIQESILMLPEEVEEVIGNKPESDILVHTAYDESTDENVMLLTSDAPEY
KPWALVIQDSNGENKIKMLSGGSGGSGGSTNLSDIIEKETGKQLVIQESI
LMLPEEVEEVIGNKPESDILVHTAYDESTDENVMLLTSDAPEYKPWALVI

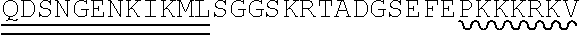

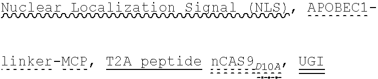

1. An RNA scaffold comprising:
(a) a tracrRNA; and
(b) an RNA motif with an extension sequence.

2. The RNA scaffold according to claim 1, wherein the scaffold further comprises a crRNA comprising a guide RNA sequence.

3. The RNA scaffold according to claim 1, wherein the scaffold comprises one or more modification(s).

4. The RNA scaffold according to claim 1, wherein the RNA motif is linked to the 3′ end of the tracrRNA via a linker.

5. (canceled)

6. The RNA scaffold according to claim 2, wherein the tracrRNA is fused to the crRNA comprising a guide RNA sequence forming a single RNA molecule.

7. (canceled)

8. The RNA scaffold according to claim 2, wherein the tracrRNA hybridizes to the crRNA via a repeat anti-repeat region.

9. The RNA scaffold according to claim 8, wherein the repeat anti-repeat region is extended.

10. The RNA scaffold according to claim 9, wherein the repeat anti-repeat region comprises an upper stem that is extended comprising a total length of 20-26 nucleotides.

11. (canceled)

12. (canceled)

13. The RNA scaffold according to claim 1, wherein the RNA scaffold comprises one or more RNA motif(s).

14. The RNA scaffold according to claim 13, wherein the one or more RNA motif(s) comprises one or more modification(s).

15. (canceled)

16. (canceled)

17. (canceled)

18. (canceled)

19. (canceled)

20. The RNA scaffold according to claim 1, wherein the extension sequence of the RNA motif comprises 2-24 nucleotides, wherein the recruiting RNA motif has a total length of 23-45 nucleotides.

21. (canceled)

22. (canceled)

23. The RNA scaffold according to claim 13, wherein the one or more RNA motif(s) is an aptamer selected from the group consisting of: MS2, Ku, PP7, SfMu and Sm7.

24. (canceled)

25. The RNA scaffold according to claim 23, wherein the MS2 aptamer is a wild-type MS2, a mutant MS2, or variants thereof.

26. The RNA scaffold according to claim 25, wherein the mutant MS2 is a C-5, F-5 hybrid and/or F-5 mutant.

27. The RNA scaffold according to claim 1, wherein the RNA motif recruits an effector module.

28. The RNA scaffold according to claim 27, wherein the effector module comprises: (i) an RNA binding domain capable of binding to the RNA motif; and (ii) an effector domain.

29. The RNA scaffold according to claim 28, wherein the effector domain is selected from: reporters, tags, molecules, proteins, particulates and nano-particles.

30. The RNA scaffold according to claim 28, wherein the effector domain is a DNA modification enzyme.

31. The RNA scaffold according to claim 30, wherein the DNA modification enzyme is selected from: AID, CDA, APOBEC1, APOBEC3A, APOBEC3B, APOBEC3C, APOBEC3D, APOBEC3F, or other APOBEC family enzymes, ADA, ADAR family enzymes, or tRNA adenosine deaminases.

32. (canceled)

33. The RNA scaffold according to claim 1, wherein the RNA motif has a sequence selected from SEQ ID NO: 21 to SEQ ID NO: 24.
